(19) United States
(12) Patent Application Publication
(10) Pub. No.: US 2025/0011450 A1
(43) Pub. Date: Jan. 9, 2025

(54) ANTIBODIES AND USES THEREOF

(71) Applicant: Genentech, Inc., South San Francisco, CA (US)

(72) Inventors: Gregory Alan LAZAR, Pacifica, CA (US); Brandon LEONARD, Pacifica, CA (US); Matthew G. ROMEI, Richmond, CA (US)

(73) Assignee: Genentech, Inc., South San Francisco, CA (US)

(21) Appl. No.: 18/749,445

(22) Filed: Jun. 20, 2024

Related U.S. Application Data
(60) Provisional application No. 63/509,744, filed on Jun. 22, 2023.

(51) Int. Cl. C07K 16/28; C12N 15/10

(52) U.S. Cl. CPC C07K 16/2878; C07K 16/2866; C12N 15/1037; C07K 2317/31; C07K 2317/35; C07K 2317/55; C07K 2317/75; C07K 2317/92

(57) ABSTRACT

Provided herein are antibodies such as human or humanized antibodies with a modified heavy chain variable (VH) domain such that the antibodies adopt a constrained conformation (e.g., an i-shaped format) upon engaging the antigen(s) the antibodies bind to, thereby confer agonistic activities. In some cases, the antibodies are full-length antibodies (e.g., IgG antibodies). In some cases, the antibodies are monovalent antibodies (e.g., Fabs). In some cases, the antibodies target receptors which require clustering for activation (e.g., TNFR superfamily receptors). In some cases, the antibodies target two antigens on a molecule that have two or more subunits (e.g., an IL-2 receptor). Also provided herein are methods of making and using such antibodies and libraries for discovering or screening such antibodies.

### CROSS-REFERENCE TO RELATED APPLICATIONS

This application claims benefit of priority to U.S. Provisional Application No. 63/509,744 filed Jun. 22, 2023, which is incorporated herein by reference in its entirety.

### SEQUENCE LISTING

The instant application contains a Sequence Listing which has been submitted electronically in XML file format and is hereby incorporated by reference in its entirety. Said XML copy, created on May 20, 2024, is named P38276-US_SL.xml and is 14,095 bytes in size.

### FIELD

The present disclosure relates to antibodies adopting a constrained conformation (e.g., i-shaped format) when engaging with antigen(s) they bind to.

### BACKGROUND

Biotherapeutic activation of target receptors can be an enormously impactful pharmacologic mechanism for the treatment of disease. For example, protein drugs that activate the erythropoietin, growth hormone, insulin, and incretin pathways illustrate the therapeutic benefit from direct agonism of cell surface receptors, in these cases by their natural cognate ligands (Thilaka, G. K. and Kumar, S. V. Apollo Medicine 2016. 13, 80-85). Correspondingly, the clinical success of these specific examples is a consequence in part of the developability of the biological ligands themselves as drug products. Yet this is not always the case, and there remain many receptor targets of high therapeutic potential for which natural ligands make good research reagents but poor drugs. Hurdles can include weak protein stability and/or solubility, complex glycosylation, unfavorable pharmacokinetics (PK) or distribution, and risk of immunogenicity and consequent risk of cross-reactivity with endogenous protein.

Monoclonal antibodies are the most prevalent and clinically successful class of biotherapeutics and generally do not suffer from the same limitations as other protein-based drugs. Despite their macromolecular complexity, antibody drugs typically possess favorable stability and solution properties, limited and well-controlled and-defined carbohydrate modification, favorable PK, and relatively low immunogenicity with little evidence of endogenous cross-reactivity. Moreover, decades of drug development experience have resulted in extensive research capabilities for discovery and optimization, and process capabilities for downstream production, purification, formulation, and delivery. Mechanistically, antibodies have demonstrated strong success as competitive inhibitors, mediators of immune effector function, delivery of toxic agents, and more recently immune redirection.

However, in some cases, antibodies are not as clinically accomplished as target activators, so-called receptor agonists or ligand mimetics. A principal challenge for this class is that the mechanisms by which natural ligands activate receptors are diverse and sometimes insufficiently understood to enable first principal design of an active agonist. For example, the ligands of most TNFRSF members induce receptor homo-trimerization when expressed in soluble form and higher order clustering when tethered to a membrane (Wajant, H. Cell Death Differ, 2015. 22, 1727-1741), and strong agonism activity has been observed for bivalent aptamers. On the other hand, many cytokine receptors require heterodimerization of two receptors in-cis in a conformationally specific manner (Waldmann, T. A. Csh Perspect Biol., 2018. 10, a028472). Therapeutic antibodies for such targets with potent agonistic activities are needed.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY OF THE APPLICATION

The present application in one aspect provides human or humanized antibodies comprising a first heavy chain variable (VH) domain and a first light chain variable (VL) domain, wherein the first VH and the first VL binds to a first target, wherein the VH domain comprises, according to Kabat numbering: 7S, 17T, 19V, 21L, 68T, 70F, 77Q, 79I, 81I, and 82aT; 7S, 17S, 19I, 21S, 68F, 70F, 77T, 79Y, 81V, and 82aS; or 14A, 19I, 39R, 43G, 57R, 74L, 75E, 77F, 82aH, 82bK, 82 cM, 84V, and 113P, wherein the antibody does not bind to HIV. In some embodiments, the VH domain of the human or humanized antibody comprises, according to Kabat numbering, 7S, 17T, 19V, 21L, 68T, 70F, 77Q, 79I, 81I, and 82aT. In some embodiments, the VH of the human or humanized antibody comprises at least one or more substitutions at the position(s) selected from 19, 21, 70, 79, and 81. In some embodiments, the VH domain of the human or humanized antibody comprises, according to Kabat numbering, 7S, 17S, 19I, 21S, 68F, 70F, 77T, 79Y, 81V, and 82aS. In some embodiments, the VH of the human or humanized antibody comprises at least one or more substitutions at the position(s) selected from 19, 68, 70, and 81. In some embodiments, the VH domain of the human or humanized antibody comprises, according to Kabat numbering, 14A, 19I, 39R, 43G, 57R, 74L, 75E, 77F, 82aH, 82bK, 82 cM, 84V, and 113P. In some embodiments, the VH of the human or humanized antibody comprises at least one or more substitutions at the position(s) selected from 14, 19, 39, 43, 74, 77, 82a, and 82b.

The present application in one aspect provides human or humanized antibodies that are derived from a reference antibody, wherein the antibody and the reference antibody both comprise a heavy chain variable (VH) domain and a light chain variable (VL) domain, wherein: the VH domain of the human or humanized antibody comprises, according to Kabat numbering, 1) at least one or more amino acid substitutions selected from the group consisting of 7S, 17T, 19V, 21L, 68T, 70F, 77Q, 79I, 81I, and 82aT, and 2) 7S, 17T, 19V, 21L, 68T, 70F, 77Q, 79I, 81I, and 82aT; the VH domain of the human or humanized antibody comprises, according to Kabat numbering, 1) at least one or more amino acid substitutions selected from the group consisting of 7S, 17S, 19I, 21S, 68F, 70F, 77T, 79Y, 81V, and 82aS, and 2) 7S, 17S, 19I, 21S, 68F, 70F, 77T, 79Y, 81V, and 82aS; or the VH domain of the human or humanized antibody comprises, according to Kabat numbering, 1) at least one or more amino acid substitutions selected from the group consisting of 14A, 19I, 39R, 43G, 57R, 74L, 75E, 77F, 82aH, 82bK, 82 cM, 84V, and 113P, and 2) 14A, 19I, 39R, 43G, 57R, 74L, 75E, 77F, 82aH, 82bK, 82 cM, 84V, and 113P, wherein the substitutions in a), b), and c) are substitutions compared to the reference antibody; wherein optionally the human or humanized antibody has increased agonistic activity relative to the reference antibody. In some embodiments, the antibody (e.g., the human or humanized antibody) does not bind to HIV. In some embodiments, the VH domain of the human or humanized antibody comprises, according to Kabat numbering, 7S, 17T, 19V, 21L, 68T, 70F, 77Q, 79I, 81I, and 82aT. In some embodiments, the VH of the human or humanized antibody comprises at least one or more substitutions at the position(s) selected from 19, 21, 70, 79, and 81. In some embodiments, the VH domain of the human or humanized antibody comprises, according to Kabat numbering, 7S, 17S, 19I, 21S, 68F, 70F, 77T, 79Y, 81V, and 82aS. In some embodiments, the VH of the human or humanized antibody comprises at least one or more substitutions at the position(s) selected from 19, 68, 70, and 81. In some embodiments, the VH domain of the human or humanized antibody comprises, according to Kabat numbering, 14A, 19I, 39R, 43G, 57R, 74L, 75E, 77F, 82aH, 82bK, 82 cM, 84V, and 113P. In some embodiments, the VH of the human or humanized antibody comprises at least one or more substitutions at the position(s) selected from 14, 19, 39, 43, 74, 77, 82a, and 82b.

In some embodiments according to any of the human or humanized antibodies described above, the antibody is a monovalent antibody. In some embodiments, the monovalent antibody is a Fab.

In some embodiments according to any of the human or humanized antibodies described above, the antibody is a F(ab′) 2.

In some embodiments according to any of the human or humanized antibodies described above, the antibody does not have a Fc domain.

In some embodiments according to some of human or humanized antibodies described above, the antibody has a Fc domain. In some embodiments, the antibody is an IgG antibody.

In some embodiments according to any of the human or humanized antibodies described above, the antibody has a modified hinge, wherein the modified hinge confers more restricted hinge flexibility.

In some embodiments according to any of the human or humanized antibodies described above, the human or humanized antibody is a monospecific antibody.

In some embodiments according to any of the human or humanized antibodies described above, the human or humanized antibody binds to a cell surface receptor.

In some embodiments according to any of the human or humanized antibodies described above, the human or humanized antibody activates a target via receptor clustering.

In some embodiments, the antibody (e.g., the human or humanized antibody) binds to one or more TNFRSF member. In some embodiments, the antibody (e.g., the human or humanized antibody) binds to OX40, CD40, 4-1BB, DR4, or DR5. In some embodiments, the antibody (e.g., the human or humanized antibody) binds to CD40, optionally wherein the human or humanized antibody is derived from an antibody selected from the group consisting of ravagalimab, dacetuzumab, giloralimab, and sotigolimab. In some embodiments, the antibody (e.g., the human or humanized antibody) binds to OX40, optionally wherein the human or humanized antibody is derived from an antibody selected from the group consisting of 3C8, 1A7, 2A3, 2B5, 2F10, 2G7, 2H5, 3F5, 3G5, and 3G8.

In some embodiments according to some of the human or humanized antibodies described above, the human or humanized antibody binds to a receptor of a cytokine. In some embodiments, the cytokine can form a complex in nature with at least two distinct receptors, which triggers downstream activity of the cytokine. In some embodiments, the antibody (e.g., the human or humanized antibody) binds to an IL-2 receptor. In some embodiments, the IL-2 receptor is IL-2RG or IL-2RB.

In some embodiments according to some of the human or humanized antibodies described above, the human or humanized antibody is a bivalent antibody comprising a second VH domain and a second VL domain binding to a second target. In some embodiments, the second VH domain comprises, according to Kabat numbering: 7S, 17T, 19V, 21L, 68T, 70F, 77Q, 79I, 81I, and 82aT; 7S, 17S, 19I, 21S, 68F, 70F, 77T, 79Y, 81V, and 82aS; or 14A, 19I, 39R, 43G, 57R, 74L, 75E, 77F, 82aH, 82bK, 82 cM, 84V, and 113P. In some embodiments, the second target is distinct from the first target. In some embodiments, the antibody (e.g., the human or humanized antibody) binds to both IL-2RG and IL-2RB. In some embodiments, the VH domain of the human of humanized antibody comprises 3 VH CDR sequences of B10, and wherein the VL domains comprises 3 VL CDR sequences of B10. In some embodiments, one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28.

The present application in another aspect provides a pharmaceutical composition comprising any of the human or humanized antibodies described above and a pharmaceutical career.

The present application in another aspect provides an isolated nucleic acid encoding any of the human or humanized antibodies described above or a fragment thereof.

The present application in another aspect provides a host cell comprising any of the nucleic acids described above.

The present application in another aspect provides a method of producing any of the human or humanized antibodies described above or a fragment thereof comprising culturing any of the host cells described above under conditions suitable for the expression of the antibody or a fragment thereof. In some embodiments, the method further comprises recovering the antibody or a fragment thereof from the host cell.

The present application in another aspect provides a method of producing an agonist antibody from a reference antibody, comprising substituting one or more amino acid residues on a heavy chain variable (VH) domain of the reference antibody to promote an i-shaped antibody format.

The present application in another aspect provides a method of producing an agonist antibody from a reference antibody, comprising: substituting one or more amino acid residues on a heavy chain variable (VH) domain of the reference antibody at position(s) according to Kabat numbering selected from 7, 17, 19, 21, 68 70, 77, 79, 81, and/or 82a, wherein the agonist antibody has a VH domain comprising 7S, 17T, 19V, 21L, 68T, 70F, 77Q, 79I, 81I, and 82aT after substitution; substituting one or more amino acid residues on a heavy chain variable (VH) domain of the reference antibody at position(s) according to Kabat numbering selected from 7, 17, 19, 21, 68 70, 77, 79, 81, and/or 82a, wherein the agonist antibody has a VH domain comprising 7S, 17S, 19I, 21S, 68F, 70F, 77T, 79Y, 81V, and 82aS after substitution; or substituting one or more amino acid residues on a heavy chain variable (VH) domain of the reference antibody at position(s) according to Kabat numbering selected from 14, 19, 39, 43, 57, 74, 75, 77, 82a, 82b, 82c, 84, and 113, wherein the agonist antibody has a VH domain comprising 14A, 19I, 39R, 43G, 57R, 74L, 75E, 77F, 82aH, 82bK, 82 cM, 84V, and 113P after substitution.

The present application in another aspect provides an agonist antibody produced by any of the methods described above.

The present application in another aspect provides a method of promoting agonistic activity of an antibody, comprising: substituting one or more amino acid residues on a heavy chain variable (VH) domain of the antibody at position(s) according to Kabat numbering selected from 7, 17, 19, 21, 68 70, 77, 79, 81, and/or 82a, wherein the VH domain after substitution comprises 7S, 17T, 19V, 21L, 68T, 70F, 77Q, 79I, 81I, and 82aT; substituting one or more amino acid residues on a heavy chain variable (VH) domain of the antibody at position(s) according to Kabat numbering selected from 7, 17, 19, 21, 68 70, 77, 79, 81, and/or 82a, wherein the VH domain after substitution comprises 7S, 17S, 19I, 21S, 68F, 70F, 77T, 79Y, 81V, and 82aS; or substituting one or more amino acid residues on a heavy chain variable (VH) domain of the antibody at position(s) according to Kabat numbering selected from 14, 19, 39, 43, 57, 74, 75, 77, 82a, 82b, 82c, 84, and 113, wherein the VH domain after substitution comprises 14A, 19I, 39R, 43G, 57R, 74L, 75E, 77F, 82aH, 82bK, 82 cM, 84V, and 113P.

The present application in another aspect provides any of the antibodies or pharmaceutical compositions described above for used as a medicament.

The present application in another aspect provides any of the antibodies or pharmaceutical compositions described above for use in treating a disease or condition.

The present application in another aspect provides uses of any of the antibodies or the pharmaceutical compositions described above in the manufacture of a medicament for treating a disease or condition.

The present application in another aspect provides a method of treating an individual having a disease or condition comprising administering to the individual an effective amount of any of the antibodies or the pharmaceutical compositions described above.

The present application in another aspect provides a library comprising polynucleotides, wherein the polynucleotides in the library encode at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering: a) 7S, 17T, 19V, 21L, 68T, 70F, 77Q, 79I, 81I, and 82aT; b) 7S, 17S, 19I, 21S, 68F, 70F, 77T, 79Y, 81V, and 82aS; or c) 14A, 19I, 39R, 43G, 57R, 74L, 75E, 77F, 82aH, 82bK, 82 cM, 84V, and 113P.

The present application in another aspect provides is a library of antibodies, comprising at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering: a) 7S, 17T, 19V, 21L, 68T, 70F, 77Q, 79I, 81I, and 82aT; b) 7S, 17S, 19I, 21S, 68F, 70F, 77T, 79Y, 81V, and 82aS; or c) 14A, 19I, 39R, 43G, 57R, 74L, 75E, 77F, 82aH, 82bK, 82 cM, 84V, and 113P. In some embodiments, the antibodies in the library are expressed or are to be expressed on the surface of one or more phages or yeast cells.

The present application in another aspect provides a method of screening an agonist antibody comprising contacting the antibodies in any of the libraries discussed above with the target or with a cell expressing the target. In some embodiments, the method further comprises assessing agonistic activity of the antibodies.

### BRIEF DESCRIPTION OF THE DRAWINGS

The drawings illustrate certain features and advantages of this disclosure. These embodiments are not intended to limit the scope of the appended claims in any manner.

FIG. 1A depicts exemplary structural representation of the domain-exchanged iAb 2G12 (left, PDB: 20QJ) and the two iAbs with an affinity interface, DH851.3 (middle, PDB: 7LU9) and DH898.1 (right, PDB: 7L6M). The light chain (LC) and heavy chain (HC) from each Fab is labeled within each structure. Insets highlight the interface between the heavy chain variable (VH) domains of the two Fabs with residues involved shown as sticks.

FIG. 1B depicts an exemplary table of amino acid residues in the VH domain predicted to contribute to the iAb conformation. VH domain residues are shown using Kabat numbering. The sequence logos below the table are based on the distribution of amino acids at the indicated residue across all human antibody sequences within the ab Ysis database. Residues boxed in a dashed line are rare mutations present in <1% of all deposited human sequences. Asterisks denote additional, non-native hydrophobic substitutions previously shown to strengthen the affinity interfaces from the DH851 and DH898 lineages.

FIGS. 2A-2C depict examples of iAb engineering. Sequence alignments of previously described iAbs [2G12 (FIG. 2A), DH851.3 (FIG. 2B), or DH898.1 (FIG. 2C)], WT anti-OX40 clones (1A7 and 3C8), and their respective iAb mutation set engrafted formats. Black boxed residues show positions where the anti-OX40 clone sequence was changed to that of the indicated iAb in order to engineer iAb formation. Boxes with an asterisk indicate additional, non-native hydrophobic substitutions previously shown to strengthen the affinity interfaces from the DH851 and DH898 lineages. In some cases, the boxed residue was the same between the original iAb and the anti-OX40 antibody, and no sequence alteration was needed. FIG. 2A discloses SEQ ID NOS 4-8, respectively, in order of appearance. FIG. 2B discloses SEQ ID NOS 9, 5, 10, 7, and 11, respectively, in order of appearance. FIG. 2C discloses 12, 5, 13, 7, and 14, respectively, in order of appearance.

FIG. 2D depicts exemplary cartoons and corresponding representative negative stain electron microscopy 2D classification images for WT, Contorsbody, iAb_{dx}, iAb_{aff1}, iAb_{aff2} formats.

FIG. 2E depicts analytical SEC chromatograms of the iAb_{aff2} anti-OX40 clones show a range of elution times.

FIG. 2F depicts a table of SEC-MALS data quantitatively characterizing each iAb_{aff2} clone as a monomer, dimer, or mixture of the two.

FIG. 2G depicts concentration dependence of SEC-MALS molecular weight for iAb_{aff2} 3C8. As the sample is diluted, the sample becomes more monomeric, demonstrating that the iAb interaction is in equilibrium.

FIG. 3A depicts OX40 agonism activity from a panel of ten anti-OX40 antibodies for the indicated antibody format. Each symbol represents a unique clone. Data is shown as fold change over an untreated control.

FIGS. 3B-3C depict OX40 agonism activity across three iAb inducing mutation sets. Individual titrations of 10 anti-OX40 clones engrafted with either the iAbdx (FIG. 3B) or affinity-based (FIG. 3C) iAb inducing mutation sets. The data are shown as fold change over an untreated control. 2A3 did not express with the iAbdx mutation set, while 1A7 did not express with either affinity-based mutation set.

FIG. 3D depicts surface plasmon resonance (SPR) affinity data comparing K_{D} (left) and normalized Rₘₐₓ (nRₘₐₓ, right) values for each anti-OX40 clone as either an iAb_{aff1} or WT IgG. The dotted gray line has a slope of 1 and indicates no change between the two formats.

FIG. 3E depicts exemplary cell surface binding to OX40+ Jurkat cells for each anti-OX40 clone comparing the EC50 values of the iAb_{aff1} and WT IgG formats. The dotted gray line has a slope of 1 and indicates no change between the two formats.

FIG. 3F depicts anti-OX40 cell binding titrations. Binding of each anti-OX40 clone as WT IgG, contorsbody and iAbaff1 to OX40+ Jurkatcells was detected by FACS with a fluorescently labeled anti-human IgG Fab.

FIG. 4A depicts the effect of antibody fragmentation on OX40 agonism activity with and without iAb_{aff1} mutation set engraftment for a single anti-OX40 clone, 3C8. F-I) TNFRSF agonism activity of various formats of clones against CD40 (F, 4 nM), 4-1BB (G, 22.2 nM), DR4 (H, 100 nM), and DR5 (I, 100 nM). Data are shown at a single concentration (indicated above in parentheses) taken from titration curves in FIG. S5. CD40 and 4-1BB agonism are shown as fold change over an untreated control, while DR4 and DR5 agonism are shown as % killing relative to an untreated control.

FIG. 5A depicts TNFRSF agonism activity of WT IgG1, IgG2 C131S, and iAb_{aff1} IgG1 clones against CD40 (4 nM). Data are shown at a single concentration (indicated above in parentheses) taken from titration curves in FIG. 5E.

FIG. 5B depicts TNFRSF agonism activity of WT and iAb_{aff1} clones against 4-1BB (22.2 nM). Data are shown at a single concentration (indicated above in parentheses) taken from titration curves in FIG. 5F.

FIG. 5C depicts TNFRSF agonism activity of WT and iAb_{aff1} clones against DR4 (100 nM). Data are shown at a single concentration (indicated above in parentheses) taken from titration curves in FIG. 5G.

FIG. 5D depicts TNFRSF agonism activity of WT and iAb_{aff1} clones against DR5 (100 nM) Data are shown at a single concentration (indicated above in parentheses) taken from titration curves in FIG. 5H.

FIGS. 5E-5H depict iAb-induced agonism across 4 TNFRSF members. Individual activity titrations are shown of various formats for antibody clones against CD40 (FIG. 5E), 4-1BB (FIG. 5F), DR4 (FIG. 5G), and DR5 (FIG. 5H). CD40 and 4-1BB agonism are shown as fold change over an untreated control, while DR4 and DR5 agonism are shown as % killing relative to an untreated control. Each anti-CD40 clone was produced as a WT human IgG1, human IgG2 C131S, and iAb_{aff1}, while all clones against other targets were only produced as a WT IgG1 and iAb_{aff1}.

FIG. 6A depicts exemplary cartoons and a legend depicting each format engineered into the anti-OX40 3C8 clone. FIGS. 3B-3F are colored according to the legend.

FIG. 6B depicts OX40 agonism activity of each format.

FIG. 6C depicts receptor-mediated internalization of each format shown as MFIx10⁴. An antibody against an irrelevant viral antigen (gD) labeled with the same pH sensitive dye is shown as a control in black.

FIG. 6D depicts TIRF microscopy max projections (grayscale) for a 12.5 second acquisition of a representative Jurkat T cell expressing OX40-mNeonGreen and treated with the indicated antibodies at 13.3 nM in solution. Insets show representative single molecule tracks as grey lines within a 2.5×2.5 um area.

FIG. 6E depicts average mean square displacement (MSD) plots for all analyzed tracks are shown for each treatment condition. The untreated control is shown in black.

FIG. 6F depicts the distribution of the average background subtracted molecular track intensity for each treatment condition. The untreated control is shown in black.

FIG. 7A depicts an exemplary schematic depicting the selection and screening process for binders to IL-2RG and IL-2RB.

FIG. 7B depicts yeast selection overview. The schematics depict selection of IL-2RG (left column) and IL-2RB (right column) binders from an in-house derived scFv library displayed on yeast. For each antigen, selection was either magnetic- or FACS-based under increasingly stringent conditions with regard to both valency and concentration, as indicated. FACS plots at 37 nM of each antigen show enrichment of binders after each round of selection.

FIG. 7C depicts plots comparing affinities determined by SPR and cell surface binding propensity for 34 anti-IL-2RG (top) and 61 anti-IL-2RB clones (bottom). Clones that block IL-2 signaling in a Jurkat reporter assay are shown as squares and non-blocking clones are shown a circle. Grey symbols indicate selected lead clones for IL-2RG and IL-2RB. All other nonlead clones are black.

FIG. 7D-7E depict the characterization of anti-IL-2RB and anti-IL-2RG antibody clones discovered by yeast display. Cell binding (FIG. 7D) and IL-2 blocking (FIG. 7E) analysis using IL-2RB and IL-2RG expressing Jurkat cells with a STAT5-luc reporter for anti-IL-2RG (top) and anti-IL-2RB (bottom) clones discovered by yeast display. For IL-2 blocking experiments, cells were first coated with 1 μM of each monospecific anti-IL-2RG or anti-IL-2RB clone for 1 hour prior to the addition of an IL-2 serial dilution. For each analysis the anti-HER2 antibody, trastuzumab, was used as a negative control and labeled with “c”. Lead clones selected for bispecific reformatting are indicated with an asterisk.

FIG. 7F depicts surface plasmon resonance analysis of anti-IL-2RB and anti-IL-2RG antibody clones.

FIG. 8A depicts epitope mapping of lead anti-IL-2RG (blue box) and anti-IL-2RB (red box) clones. Epitopes on IL-2RG and IL-2RB are shown in dark grey. For reference, the IL-2 binding site is highlighted based on the ternary complex (black box, PDB: 2ERJ). In each image, the IL-2RG and IL-2RB structures are shown on the left and right, respectively. A 90° rotated image of the crystal structure is shown to further depict epitope residues.

FIG. 9A depicts heatmaps showing fold IL-2 pathway agonism in a Jurkat-STAT5 luciferase reporter cell line expressing IL-2RG and IL-2RB. Bispecific WT IgG (left), contorsbodies (middle), and iAbs (right) were tested at 100 nM concentration with the indicated anti-IL-2RG and anti-IL-2RB clone combinations. Activity of recombinant IL-2 is shown for comparison. An X for the G23/B65 iAb indicates no expression.

FIG. 9B depicts concentration-dependent activity of lead constrained IL-2 pathway agonists and corresponding WT IgG controls in the Jurkat reporter assay. A legend showing symbols for each line is shown below and an antibody against an irrelevant viral antigen (gD) is shown as a control.

FIG. 9C depicts an exemplary schematic (left) and plot (right) of an IL-2RG/IL-2RB bridging ELISA. A legend showing symbols for each line is shown below and an antibody against an irrelevant viral antigen (gD) is shown as a control.

FIG. 10A depicts activity of lead constrained IL-2 pathway agonists and corresponding WT IgG controls in primary NK cells (left), and primary CD8 T cells (right). A legend showing symbols for each line is shown below and an antibody against an irrelevant viral antigen (gD) is shown as a control.

FIG. 10B depicts hierarchical clustering of lead constrained IL-2 pathway agonists and corresponding WT IgG controls based on altered gene expression in primary CD8 T cells as determined by RNA sequencing. The heat map shows gene expression changes under each condition relative to the gD control for the 40 most significantly down-regulated (left) and up-regulated (right) genes by IL-2.

### DETAILED DESCRIPTION

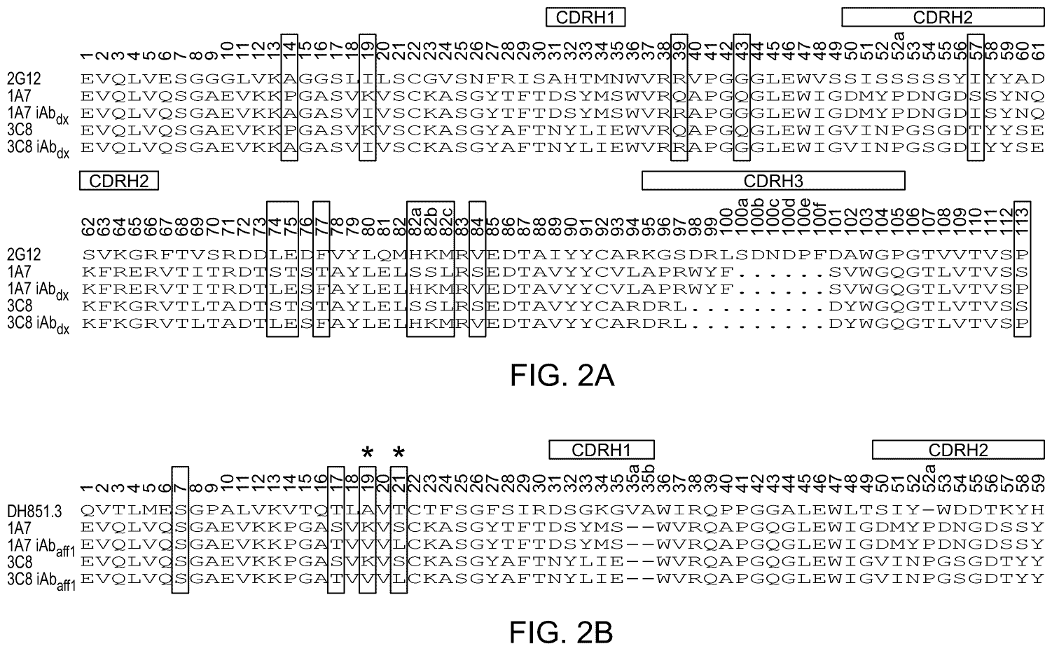

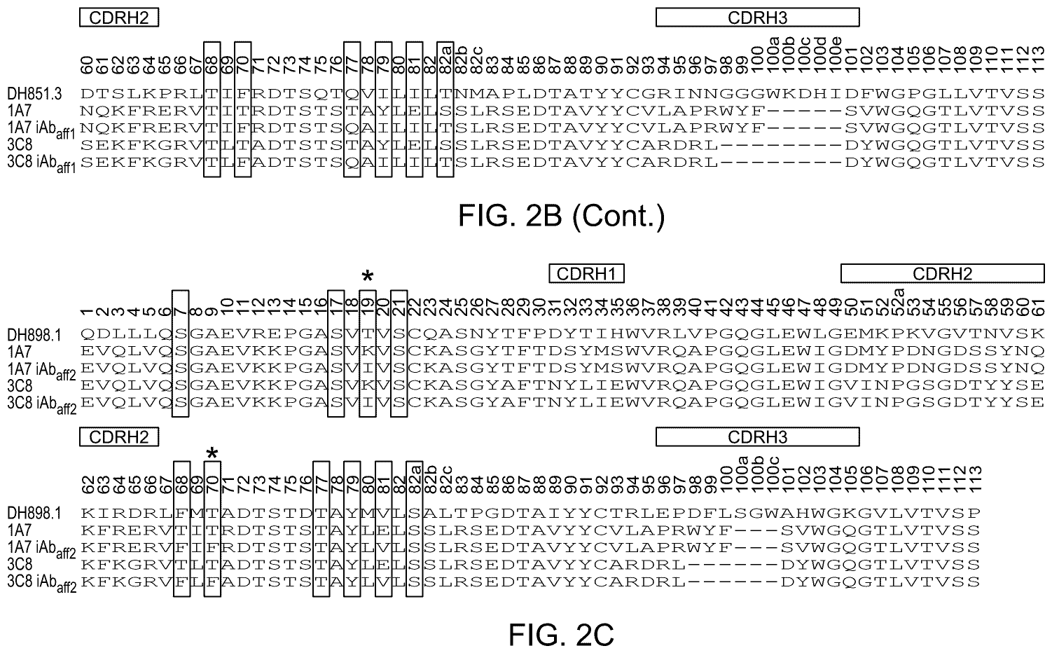

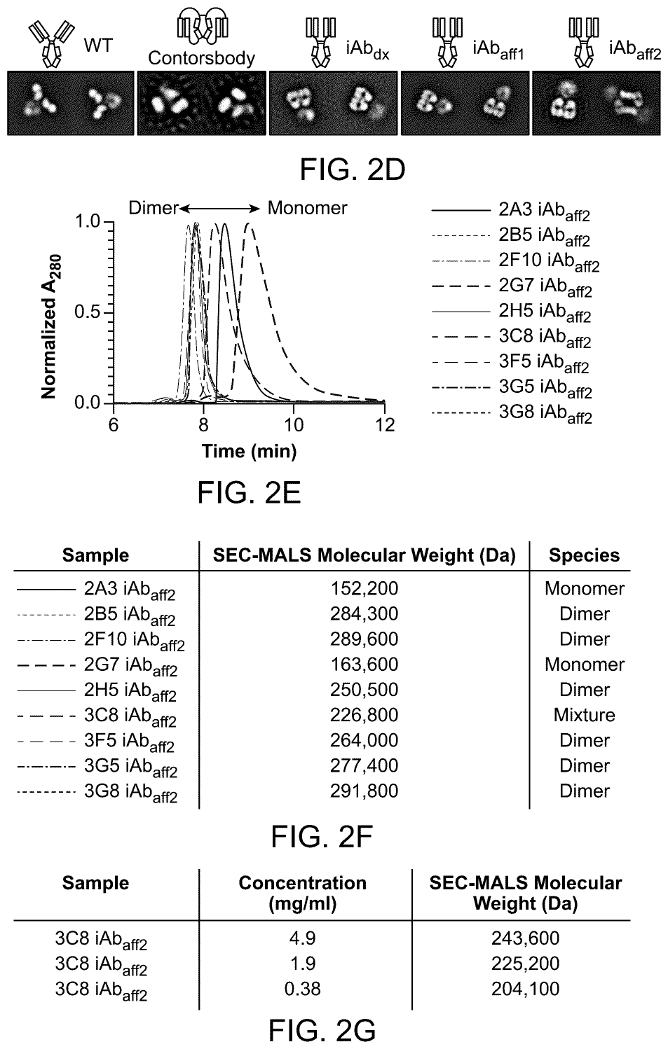

The present application provides antibodies with agonistic activities and methods of preparing such antibodies. Without being bound to the theory, the present application is at least in part base upon inventors' insightful findings that by modulating antibody geometry such that two antigen-binding domains of one or more antibodies that target one or more antigens (e.g., receptors that require a homo-multimerization or hetero-multimerization for activation) are conformationally constrained via a non-covalent association (e.g., via affinity interface between two VH domains), the antibody or antibodies can engage the antigen(s) and activate downstream signaling. In some embodiments, the antibody or antibodies adopt an i-shape format upon engaging with antigen(s). See e.g., FIG. 2D for images of a compact i-shape of exemplary antibodies (see the representative electron microscopy images of iAb_{dx}, iAb_{aff1} and iAb_{aff2} as compared to WT). Strikingly, the inventors found that not only a classic IgG antibody with two Fab arms (such as iAb_{dx}, iAb_{aff1} and iAb_{aff2} as demonstrated in FIG. 2D), but also monovalent formats with one antigen-binding domain (e.g., a monomeric Fab) can enable intrinsic agonist activity. See, e.g., Example 4.

In some embodiments, the present application provides human or humanized antibody comprising a first heavy chain variable (VH) domain and a first light chain variable (VL) domain, wherein the first VH and the first VL binds to a first target, wherein the VH domain comprises, according to Kabat numbering: 7S, 17T, 19V, 21L, 68T, 70F, 77Q, 79I, 81I, and 82aT; 7S, 17S, 19I, 21S, 68F, 70F, 77T, 79Y, 81V, and 82aS; or 14A, 19I, 39R, 43G, 57R, 74L, 75E, 77F, 82aH, 82bK, 82 cM, 84V, and 113P. In some embodiments, the antibody does not bind to HIV. In some embodiments, the antibody (e.g., the human or humanized antibody) is derived from a reference antibody, and wherein the humanized antibody comprises at least one or more amino acid substitution selected from a) 7S, 17T, 19V, 21L, 68T, 70F, 77Q, 79I, 81I, and 82aT; b) 7S, 17S, 19I, 21S, 68F, 70F, 77T, 79Y, 81V, and 82aS; or c) 14A, 19I, 39R, 43G, 57R, 74L, 75E, 77F, 82aH, 82bK, 82 cM, 84V, and 113P, wherein the substitutions in a), b), and c) are substitutions compared to the reference antibody. In some embodiments, the antibody (e.g., the human or humanized antibody) has increased agonistic activity relative to the reference antibody. In some embodiments, the antibody is a monovalent antibody (e.g., a Fab). In some embodiments, the antibody is an IgG antibody. In some embodiments, the antibody (e.g., the human or humanized antibody) binds to a cell surface receptor (e.g., a TNFRSF member, e.g., a cytokine receptor).

Pharmaceutical compositions comprising the antibodies, isolated nucleic acids encoding the antibodies, vectors and host cells comprising the isolated nucleic acids, methods of producing the antibodies, methods of promoting agonistic activity of an antibody, methods of treatment by administering the antibodies are contemplated. Libraries comprising such antibodies or polynucleotides encoding such antibodies, as well as methods of screening such antibodies are also contemplated.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); and The Antibodies (M. Zanetti and J. D. Capra, eds., Harwood Academic Publishers, 1995).

### II. DEFINITIONS

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “agonist”, “agonistic”, “agonism” or “agonize” as used herein in general refers to a binding molecule (e.g., an antigen binding polypeptide or antigen binding complex) which binds to a receptor on the surface of a cell and is capable of initiating/mimicking/stimulating a reaction or activity that is similar to or the same as that initiated/mimicked/stimulated by the receptor's natural ligand. In exemplary embodiments, an agonist as described herein is capable of inducing/augmenting/enhancing/stimulating the activation of a signal transduction pathway associated with the receptor.

The terms “an antibody that binds to a target” refer to an antibody that is capable of binding the target with sufficient affinity such that the antibody is useful as a diagnostic and/or therapeutic agent in targeting the target. In one aspect, the extent of binding of an antibody to an unrelated, non-target protein is less than about 10% of the binding of the antibody to target as measured, e.g., by surface plasmon resonance (SPR). In certain aspects, an antibody that binds to target has a dissociation constant (K_{D}) of ≤1 μM, ≤100 nM, ≤10 nM, ≤1 nM, ≤0.1 nM, ≤ 0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to target when the antibody has a K_{D} of 1 μM or less. In certain aspects, an antibody binds to an epitope of the target that is conserved among target from different species.

The term “antibody” herein is used in the broadest sense and encompasses molecules that have various antibody structures that exhibit a desired antigen-binding activity, including but not limited to monoclonal antibodies, polyclonal antibodies, multispecific antibodies (e.g., bispecific antibodies), antibody fragments, and fusion proteins comprising an antibody fragment.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “epitope” denotes the site on an antigen, either proteinaceous or non-proteinaceous, to which an antibody binds. Epitopes can be formed both from contiguous amino acid stretches (linear epitope) or from non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Competitive binding can be used to determine whether an antibody competes for binding with a reference antibody that binds to the same target. For example, an “antibody that competes for binding with a reference antibody” refers to an antibody that blocks binding of the reference antibody to its antigen in a competition assay by 50% or more, and conversely, the reference antibody blocks binding of the antibody to its antigen in a competition assay by 50% or more. Also, for example, to determine if an antibody competes for binding with a reference antibody, the reference antibody is allowed to bind to the target under saturating conditions. After removal of the excess of the reference antibody, the ability of an antibody in question to bind to the target is assessed. If the antibody is able to bind to the target after saturation binding of the reference antibody, it can be concluded that the antibody in question binds to a different epitope than the reference antibody. But, if the antibody in question is not able to bind to the target after saturation binding of the reference antibody, then the antibody in question may bind to the same epitope as the epitope bound by the reference antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e., with both of the antibodies being the saturating antibody. If, in both set-ups, only the first (saturating) antibody is capable of binding to the target, then it can be concluded that the antibody in question and the reference antibody compete for binding to the target.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₁ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG₁, IgG₂, IgG₃, or IgG₄ and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the application in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an agonist antibody or a fragment thereof” refers to one or more nucleic acid molecules encoding one or more polypeptides of the agonist antibodies or fragment thereof, including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, which contain information about the indications, usage, dosage, administration, combination therapy, contraindications and/or warnings concerning the use of such therapeutic products.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the application are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

It should be noted that, as used in the specification and the appended claims, the singular forms “a,” “an,” and “the” include plural referents unless the context clearly dictates otherwise.

### III. COMPOSITIONS AND METHODS

### A. Antibodies

The present application provides antibodies (such as agonistic antibodies, such as agonistic human or humanized antibodies) with conformationally constrained association between two antigen-binding domains e.g., via a non-covalent association, e.g., via affinity interface between two VH domains, e.g., via disulfide linkages between two Fabs or two VH domains. In some embodiments, the antibody or antibodies adopt an i-shape format upon engaging with antigen(s). See e.g., Cell. 2021 May 27; 184 (11): 2955-2972.e25. In some embodiments, the two antigen-binding domains are from one antibody (e.g., two Fab arms in an IgG antibody). In some embodiments, the two antigen-binding domains are from two antibodies (e.g., two separate Fab molecules). In some embodiments, the two antigen-binding domains bind to a same antigen (e.g., a TNFRSF member). In some embodiments, the two antigen-binding domains bind to two distinct antigens (e.g., two antigens associated with a receptor comprising two or more distinct subunits).

In some embodiments, there is provided a human or humanized antibody comprising a first antigen binding domain comprising a first heavy chain variable (VH) domain and a first light chain variable (VL) domain, wherein the first antigen binding domain to a first target, wherein the VH domain comprises, according to Kabat numbering: 7S, 17T, 19V, 21L, 68T, 70F, 77Q, 79I, 81I, and 82aT, wherein the human or humanized antibody does not bind to HIV. In some embodiments, the antibody is a monovalent antibody (e.g., a Fab). In some embodiments, the antibody is a bivalent antibody (e.g., a F(ab′)₂). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG₂ hinge domain. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

In some embodiments, there is provided a human or humanized antibody comprising a first antigen binding domain comprising a first heavy chain variable (VH) domain and a first light chain variable (VL) domain, wherein the first antigen binding domain to a first target, wherein the VH domain comprises, according to Kabat numbering: 7S, 17S, 19I, 21S, 68F, 70F, 77T, 79Y, 81V, and 82aS, wherein the human or humanized antibody does not bind to HIV. In some embodiments, the antibody is a monovalent antibody (e.g., a Fab). In some embodiments, the antibody is a bivalent antibody (e.g., a F(ab′) 2). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG₂ hinge domain. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

In some embodiments, there is provided a human or humanized antibody comprising a first antigen binding domain comprising a first heavy chain variable (VH) domain and a first light chain variable (VL) domain, wherein the first antigen binding domain to a first target, wherein the VH domain comprises, according to Kabat numbering: 14A, 19I, 39R, 43G, 57R, 74L, 75E, 77F, 82aH, 82bK, 82 cM, 84V, and 113P, wherein the human or humanized antibody does not bind to HIV. In some embodiments, the antibody is a monovalent antibody (e.g., a Fab). In some embodiments, the antibody is a bivalent antibody (e.g., a F(ab′) 2). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG2 hinge domain. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

In some embodiments, there is provided a human or humanized antibody comprising a) a first antigen binding domain comprising a first heavy chain variable (VH) domain and a first light chain variable (VL) domain, and b) a second antigen binding domain comprising a second VH domain and a second VL domain, wherein the first antigen binding domain binds to a first target and the second antigen binding domain binds to a second target, wherein the both the first VH domain and the second VH comprise, according to Kabat numbering: 7S, 17T, 19V, 21L, 68T, 70F, 77Q, 79I, 81I, and 82aT, wherein the human or humanized antibody does not bind to HIV. In some embodiments, the first target and the second target are the same target. In some embodiments, the first target and the second target bind to the same epitope of the target. In some embodiments, the first target and the second target bind to two distinct epitopes of the target. In some embodiments, the first target and the second target bind to two distinct targets. In some embodiments, the two distinct targets are two subunits of a molecule that requires or involves clustering or multimerization of the two subunits for activation of the downstream signaling (e.g., a cytokine receptor, e.g., an IL-2 receptor). In some embodiments, the two distinct targets are two molecules that are involved in a complex (e.g., a complex on a cell surface, e.g., T cell receptor complex), the formation of which confers activation of a signaling pathway. In some embodiments, the two distinct targets comprise one or more members of tumor necrosis factor receptor superfamily (TNFRSF). In some embodiments, the antibody is a bivalent antibody (e.g., a F(ab′)₂). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG2 hinge domain.

In some embodiments, there is provided a human or humanized antibody comprising a) a first antigen binding domain comprising a first heavy chain variable (VH) domain and a first light chain variable (VL) domain, and b) a second antigen binding domain comprising a second VH domain and a second VL domain, wherein the first antigen binding domain binds to a first target and the second antigen binding domain binds to a second target, wherein the both the first VH domain and the second VH comprise, according to Kabat numbering: 7S, 17S, 19I, 21S, 68F, 70F, 77T, 79Y, 81V, and 82aS, wherein the human or humanized antibody does not bind to HIV. In some embodiments, the first target and the second target are the same target. In some embodiments, the first target and the second target bind to the same epitope of the target. In some embodiments, the first target and the second target bind to two distinct epitopes of the target. In some embodiments, the first target and the second target bind to two distinct targets. In some embodiments, the two distinct targets are two subunits of a molecule that requires or involves clustering or multimerization of the two subunits for activation of the downstream signaling (e.g., a cytokine receptor, e.g., an IL-2 receptor). In some embodiments, the two distinct targets are two molecules that are involved in a complex (e.g., a complex on a cell surface, e.g., T cell receptor complex), the formation of which confers activation of a signaling pathway. In some embodiments, the two distinct targets comprise one or more members of tumor necrosis factor receptor superfamily (TNFRSF). In some embodiments, the antibody is a bivalent antibody (e.g., a F(ab′)₂). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG2 hinge domain.

In some embodiments, there is provided a human or humanized antibody comprising a) a first antigen binding domain comprising a first heavy chain variable (VH) domain and a first light chain variable (VL) domain, and b) a second antigen binding domain comprising a second VH domain and a second VL domain, wherein the first antigen binding domain binds to a first target and the second antigen binding domain binds to a second target, wherein the both the first VH domain and the second VH comprise, according to Kabat numbering: 14A, 19I, 39R, 43G, 57R, 74L, 75E, 77F, 82aH, 82bK, 82 cM, 84V, and 113P, wherein the human or humanized antibody does not bind to HIV. In some embodiments, the first target and the second target are the same target. In some embodiments, the first target and the second target bind to the same epitope of the target. In some embodiments, the first target and the second target bind to two distinct epitopes of the target. In some embodiments, the first target and the second target bind to two distinct targets. In some embodiments, the two distinct targets are two subunits of a molecule that requires or involves clustering or multimerization of the two subunits for activation of the downstream signaling (e.g., a cytokine receptor, e.g., an IL-2 receptor). In some embodiments, the two distinct targets are two molecules that are involved in a complex (e.g., a complex on a cell surface, e.g., T cell receptor complex), the formation of which confers activation of a signaling pathway. In some embodiments, the two distinct targets comprise one or more members of tumor necrosis factor receptor superfamily (TNFRSF). In some embodiments, the antibody is a bivalent antibody (e.g., a F(ab′)₂). In some embodiments, the antibody has a Fc domain, optionally wherein the antibody has an IgG Fc domain, further optionally wherein the antibody comprises L234A, L235A and/or P329G. In some embodiments, the antibody has a modified hinge, wherein the modified hinge confers more restricted hinge flexibility. In some embodiments, the antibody has a hinge derived from an IgG2 hinge domain.

In some embodiments, there is provided a human or humanized antibody comprising a first antigen binding domain comprising a first heavy chain variable (VH) domain and a first light chain variable (VL) domain, wherein the first antigen binding domain to a member of tumor necrosis factor receptor superfamily (TNFRSF), wherein the VH domain comprises, according to Kabat numbering: 7S, 17T, 19V, 21L, 68T, 70F, 77Q, 79I, 81I, and 82aT. In some embodiments, the member of TNFRSF is selected from the group consisting of OX40, CD40, 4-1BB, DR4, or DR5. In some embodiments, the human or humanized antibody binds to CD40 (e.g., CD40), optionally wherein the human or humanized antibody is derived from an antibody selected from the group consisting of CP-870,893 (RO70099789), SGN-40, selicrelumab, dacetuzumab, Chi Lob 7/4, APX005M, ADC-1013, CDX-1140, SEA-CD40, ravagalimab, giloralimab, and sotigolimab. In some embodiments, the human or humanized antibody binds to OX40 (e.g., human OX40), optionally wherein the human or humanized antibody is derived from an antibody selected from the group consisting of 3C8, 1A7, 2A3, 2B5, 2F10, 2G7, 2H5, 3F5, 3G5, 3G8, HFB301001, FS120, INBRX-106, BGB-A445, PF-04518600, MEDI6469, MEDI0562, ABBV-368, FS120, INCAGN01949, BMS986178, PF04518600, GSK3174998, and SL-279252. In some embodiments, the human or humanized antibody binds to DR4 (e.g., human DR4), optionally wherein the human or humanized antibody is derived from an antibody selected from the group consisting of HLX56, mapatumumab, m921/922, 4H6, 4G7, AY4, and TR1-mAbs. In some embodiments, the human or humanized antibody binds to DR4 (e.g., human DR5), optionally wherein the human or humanized antibody is derived from an antibody selected from the group consisting of conatumumab, drozitumab, DS-8273a, KTRM2, lexatumumab, tigatuzumab, zaptuzumab, inbrx-109, LaDR5, LBy135, mDRA6, WD1, zaptuximab, HMCAZ5, and AD5.10. In some embodiments, the antibody further comprises a second antigen binding domain comprising a second VH domain and a second VL domain, wherein the second VH domain also comprises, according to Kabat numbering: 7S, 17T, 19V, 21L, 68T, 70F, 77Q, 79I, 81I, and 82aT. In some embodiments, the second antigen binding domain binds to the same target. In some embodiments, the second antigen binding domain binds to a distinct target (e.g., a checkpoint inhibitor, e.g., a PD-1, PD-L1 or a CTLA-4, e.g., a T cell receptor, e.g., a CD3, CD4, or CD8). In some embodiments, the second antigen binding domain binds to a second member of the TNFRSF. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG2 hinge region.

In some embodiments, there is provided a human or humanized antibody comprising a first antigen binding domain comprising a first heavy chain variable (VH) domain and a first light chain variable (VL) domain, wherein the first antigen binding domain to a member of tumor necrosis factor receptor superfamily (TNFRSF), wherein the VH domain comprises, according to Kabat numbering: 7S, 17S, 19I, 21S, 68F, 70F, 77T, 79Y, 81V, and 82aS. In some embodiments, the member of TNFRSF is selected from the group consisting of OX40, CD40, 4-1BB, DR4, or DR5. In some embodiments, the human or humanized antibody binds to CD40 (e.g., CD40), optionally wherein the human or humanized antibody is derived from an antibody selected from the group consisting of CP-870,893 (RO70099789), SGN-40, selicrelumab, dacetuzumab, Chi Lob 7/4, APX005M, ADC-1013, CDX-1140, SEA-CD40, ravagalimab, giloralimab, and sotigolimab. In some embodiments, the human or humanized antibody binds to OX40 (e.g., human OX40), optionally wherein the human or humanized antibody is derived from an antibody selected from the group consisting of 3C8, 1A7, 2A3, 2B5, 2F10, 2G7, 2H5, 3F5, 3G5, 3G8, HFB301001, FS120, INBRX-106, BGB-A445, PF-04518600, MEDI6469, MEDI0562, ABBV-368, FS120, INCAGN01949, BMS986178, PF04518600, GSK3174998, and SL-279252. In some embodiments, the human or humanized antibody binds to DR4 (e.g., human DR4), optionally wherein the human or humanized antibody is derived from an antibody selected from the group consisting of HLX56, mapatumumab, m921/922, 4H6, 4G7, AY4, and TR1-mAbs. In some embodiments, the human or humanized antibody binds to DR4 (e.g., human DR5), optionally wherein the human or humanized antibody is derived from an antibody selected from the group consisting of conatumumab, drozitumab, DS-8273a, KTRM2, lexatumumab, tigatuzumab, zaptuzumab, inbrx-109, LaDR5, LBy135, mDRA6, WD1, zaptuximab, HMCAZ5, and AD5.10. In some embodiments, the antibody further comprises a second antigen binding domain comprising a second VH domain and a second VL domain, wherein the second VH domain also comprises, according to Kabat numbering: 7S, 17S, 19I, 21S, 68F, 70F, 77T, 79Y, 81V, and 82aS. In some embodiments, the second antigen binding domain binds to the same target. In some embodiments, the second antigen binding domain binds to a distinct target (e.g., a checkpoint inhibitor, e.g., a PD-1, PD-L1 or a CTLA-4, e.g., a T cell receptor, e.g., a CD3, CD4, or CD8). In some embodiments, the second antigen binding domain binds to a second member of the TNFRSF. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG₂ hinge region.

In some embodiments, there is provided a human or humanized antibody comprising a first antigen binding domain comprising a first heavy chain variable (VH) domain and a first light chain variable (VL) domain, wherein the first antigen binding domain to a member of tumor necrosis factor receptor superfamily (TNFRSF), wherein the VH domain comprises, according to Kabat numbering: 14A, 19I, 39R, 43G, 57R, 74L, 75E, 77F, 82aH, 82bK, 82 cM, 84V, and 113P. In some embodiments, the member of TNFRSF is selected from the group consisting of OX40, CD40, 4-1BB, DR4, or DR5. In some embodiments, the human or humanized antibody binds to CD40 (e.g., CD40), optionally wherein the human or humanized antibody is derived from an antibody selected from the group consisting of CP-870,893 (RO70099789), SGN-40, selicrelumab, dacetuzumab, Chi Lob 7/4, APX005M, ADC-1013, CDX-1140, SEA-CD40, ravagalimab, giloralimab, and sotigolimab. In some embodiments, the human or humanized antibody binds to OX40 (e.g., human OX40), optionally wherein the human or humanized antibody is derived from an antibody selected from the group consisting of 3C8, 1A7, 2A3, 2B5, 2F10, 2G7, 2H5, 3F5, 3G5, 3G8, HFB301001, FS120, INBRX-106, BGB-A445, PF-04518600, MEDI6469, MEDI0562, ABBV-368, FS120, INCAGN01949, BMS986178, PF04518600, GSK3174998, and SL-279252. In some embodiments, the human or humanized antibody binds to DR4 (e.g., human DR4), optionally wherein the human or humanized antibody is derived from an antibody selected from the group consisting of HLX56, mapatumumab, m921/922, 4H6, 4G7, AY4, and TR1-mAbs. In some embodiments, the human or humanized antibody binds to DR4 (e.g., human DR5), optionally wherein the human or humanized antibody is derived from an antibody selected from the group consisting of conatumumab, drozitumab, DS-8273a, KTRM2, lexatumumab, tigatuzumab, zaptuzumab, inbrx-109, LaDR5, LBy135, mDRA6, WD1, zaptuximab, HMCAZ5, and AD5.10. In some embodiments, the antibody further comprises a second antigen binding domain comprising a second VH domain and a second VL domain, wherein the second VH domain also comprises, according to Kabat numbering: 14A, 19I, 39R, 43G, 57R, 74L, 75E, 77F, 82aH, 82bK, 82 cM, 84V, and 113P. In some embodiments, the second antigen binding domain binds to the same target. In some embodiments, the second antigen binding domain binds to a distinct target (e.g., a checkpoint inhibitor, e.g., a PD-1, PD-L1 or a CTLA-4, e.g., a T cell receptor, e.g., a CD3, CD4, or CD8). In some embodiments, the second antigen binding domain binds to a second member of the TNFRSF. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG₂ hinge region.

In some embodiments, there is provided a human or humanized antibody comprising a first antigen binding domain comprising a first heavy chain variable (VH) domain and a first light chain variable (VL) domain, wherein the first antigen binding domain to a receptor of a cytokine, wherein the VH domain comprises, according to Kabat numbering: 7S, 17T, 19V, 21L, 68T, 70F, 77Q, 79I, 81I, and 82aT. In some embodiments, the cytokine can form a complex in nature with at least two distinct receptors or two subunits of a receptor, which triggers downstream activity of the cytokine. In some embodiments, the human or humanized antibody binds to an IL-2 receptor. In some embodiments, the IL-2 receptor is IL-2RG or IL-2RB. In some embodiments, the antibody further comprises a second antigen binding domain comprising a second VH domain and a second VL domain, wherein the second VH domain also comprises, according to Kabat numbering: 7S, 17T, 19V, 21L, 68T, 70F, 77Q, 79I, 81I, and 82aT. In some embodiments, the first and second antigen binding domain binds to IL-2RG and IL-2RB, respectively. In some embodiments, the VH domain of the human of humanized antibody comprises 3 VH CDR sequences of B10, and wherein the VL domains comprises 3 VL CDR sequences of B10. In some embodiments, one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG₂ hinge region.

In some embodiments, there is provided a human or humanized antibody comprising a first antigen binding domain comprising a first heavy chain variable (VH) domain and a first light chain variable (VL) domain, wherein the first antigen binding domain to a receptor of a cytokine, wherein the VH domain comprises, according to Kabat numbering: 7S, 17S, 19I, 21S, 68F, 70F, 77T, 79Y, 81V, and 82aS. In some embodiments, the cytokine can form a complex in nature with at least two distinct receptors or two subunits of a receptor, which triggers downstream activity of the cytokine. In some embodiments, the human or humanized antibody binds to an IL-2 receptor. In some embodiments, the IL-2 receptor is IL-2RG or IL-2RB. In some embodiments, the antibody further comprises a second antigen binding domain comprising a second VH domain and a second VL domain, wherein the second VH domain also comprises, according to Kabat numbering: 7S, 17S, 19I, 21S, 68F, 70F, 77T, 79Y, 81V, and 82aS. In some embodiments, the first and second antigen binding domain binds to IL-2RG and IL-2RB, respectively. In some embodiments, the VH domain of the human of humanized antibody comprises 3 VH CDR sequences of B10, and wherein the VL domains comprises 3 VL CDR sequences of B10. In some embodiments, one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG₂ hinge region.

In some embodiments, there is provided a human or humanized antibody comprising a first antigen binding domain comprising a first heavy chain variable (VH) domain and a first light chain variable (VL) domain, wherein the first antigen binding domain to a receptor of a cytokine, wherein the VH domain comprises, according to Kabat numbering: 14A, 19I, 39R, 43G, 57R, 74L, 75E, 77F, 82aH, 82bK, 82 cM, 84V, and 113P. In some embodiments, the cytokine can form a complex in nature with at least two distinct receptors or two subunits of a receptor, which triggers downstream activity of the cytokine. In some embodiments, the human or humanized antibody binds to an IL-2 receptor. In some embodiments, the IL-2 receptor is IL-2RG or IL-2RB. In some embodiments, the antibody further comprises a second antigen binding domain comprising a second VH domain and a second VL domain, wherein the second VH domain also comprises, according to Kabat numbering: 14A, 19I, 39R, 43G, 57R, 74L, 75E, 77F, 82aH, 82bK, 82 cM, 84V, and 113P. In some embodiments, the first and second antigen binding domain binds to IL-2RG and IL-2RB, respectively. In some embodiments, the VH domain of the human of humanized antibody comprises 3 VH CDR sequences of B10, and wherein the VL domains comprises 3 VL CDR sequences of B10. In some embodiments, one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG₂ hinge region.

In some embodiments, there is provided a human or humanized antibody that is derived from a reference antibody, wherein the antibody and the reference antibody both comprise a first antigen binding domain comprising a first heavy chain variable (VH) domain and a first light chain variable (VL) domain, wherein: the VH domain of the human or humanized antibody comprises, according to Kabat numbering, 1) at least one or more amino acid substitutions selected from the group consisting of 7S, 17T, 19V, 21L, 68T, 70F, 77Q, 79I, 81I, and 82aT as compared to the reference antibody, and 2) 7S, 17T, 19V, 21L, 68T, 70F, 77Q, 79I, 81I, and 82aT, wherein optionally the human or humanized antibody has increased agonistic activity relative to the reference antibody. In some embodiments, the antibody comprises or is a monovalent antibody (e.g., a Fab). In some embodiments, the antibody comprises a second antigen binding domain comprising a second VH domain and a second VL domain, optionally wherein the second VH also comprises, according to Kabat numbering, 1) at least one or more amino acid substitutions selected from the group consisting of 7S, 17T, 19V, 21L, 68T, 70F, 77Q, 79I, 81I, and 82aT as compared to the reference antibody, and 2) 7S, 17T, 19V, 21L, 68T, 70F, 77Q, 79I, 81I, and 82aT. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG2 hinge region. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

In some embodiments, there is provided a human or humanized antibody that is derived from a reference antibody, wherein the antibody and the reference antibody both comprise a first antigen binding domain comprising a first heavy chain variable (VH) domain and a first light chain variable (VL) domain, wherein: the VH domain of the human or humanized antibody comprises, according to Kabat numbering, 1) at least one or more amino acid substitutions selected from the group consisting of 7S, 17S, 19I, 21S, 68F, 70F, 77T, 79Y, 81V, and 82aS, and 2) 7S, 17S, 19I, 21S, 68F, 70F, 77T, 79Y, 81V, and 82aS, wherein optionally the human or humanized antibody has increased agonistic activity relative to the reference antibody. In some embodiments, the antibody comprises or is a monovalent antibody (e.g., a Fab). In some embodiments, the antibody comprises a second antigen binding domain comprising a second VH domain and a second VL domain, optionally wherein the second VH also comprises, according to Kabat numbering, 1) at least one or more amino acid substitutions selected from the group consisting of 7S, 17S, 19I, 21S, 68F, 70F, 77T, 79Y, 81V, and 82aS, and 2) 7S, 17S, 19I, 21S, 68F, 70F, 77T, 79Y, 81V, and 82aS. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG2 hinge region. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

In some embodiments, there is provided a human or humanized antibody that is derived from a reference antibody, wherein the antibody and the reference antibody both comprise a first antigen binding domain comprising a first heavy chain variable (VH) domain and a first light chain variable (VL) domain, wherein: the VH domain of the human or humanized antibody comprises, according to Kabat numbering, 1) at least one or more amino acid substitutions selected from the group consisting of 14A, 19I, 39R, 43G, 57R, 74L, 75E, 77F, 82aH, 82bK, 82 cM, 84V, and 113P, and 2) 14A, 19I, 39R, 43G, 57R, 74L, 75E, 77F, 82aH, 82bK, 82 cM, 84V, and 113P, wherein optionally the human or humanized antibody has increased agonistic activity relative to the reference antibody. In some embodiments, the antibody comprises or is a monovalent antibody (e.g., a Fab). In some embodiments, the antibody comprises a second antigen binding domain comprising a second VH domain and a second VL domain, optionally wherein the second VH also comprises, according to Kabat numbering, 1) at least one or more amino acid substitutions selected from the group consisting of 14A, 19I, 39R, 43G, 57R, 74L, 75E, 77F, 82aH, 82bK, 82 cM, 84V, and 113P, and 2) 14A, 19I, 39R, 43G, 57R, 74L, 75E, 77F, 82aH, 82bK, 82 cM, 84V, and 113P. In some embodiments, the antibody is a F(ab′)₂. In some embodiments, the antibody does not have a Fc domain. In some embodiments, the antibody has a Fc domain, optionally wherein the Fc domain is derived from an IgG Fc domain, further optionally wherein the IgG Fc domain comprises L234A, L235A and P329G. In some embodiments, the antibody is an IgG antibody. In some embodiments, the antibody has a modified hinge, wherein the modified hinge confers more restricted hinge flexibility. In some embodiments, the antibody has a IgG2 hinge region. In some embodiments, the human or humanized antibody binds to a cell surface receptor. In some embodiments, the human or humanized antibody activates a target via clustering or multimerization of the target. In some embodiments, the human or humanized antibody is or comprises a monospecific antibody. In some embodiments, the human or humanized antibody is or comprises a multispecific antibody.

The antibodies described herein can be monoclonal antibodies (including full-length monoclonal antibodies), polyclonal antibodies, multispecific antibodies (e.g., bispecific antibodies), and antibody fragments so long as they exhibit the desired biological activity (e.g., binding to the target). In some embodiments, the antibody is a monovalent antibody. In some embodiments, the antibody is a multivalent (e.g., bivalent) antibody.

In some aspects, the antibody is or comprises a full-length antibody. In some embodiments, the antibody is an intact IgA, IgG, IgM, IgD, IgE antibody or other antibody class or isotype as defined herein.

In some aspects, the antibody is chimeric, human, partially humanized, fully humanized, or semi-synthetic. Antibodies and/or antibody fragments described in may be derived from murine antibodies, rabbit antibodies, human antibodies, fully humanized antibodies, camelid antibody variable domains and humanized versions, shark antibody variable domains and humanized versions, and camelized antibody variable domains.

In some aspects, the antibody comprises an Fc fragment. In some aspects, the Fc fragment is selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG.

In some embodiments, the target antigen is a molecule (e.g., a receptor) that requires a clustering or multimerization for activation.

1. Constrained Conformation (e.g., i-Shape Format)

The antibodies described herein are modulated such that at least some of the antigen-binding domains of the antibodies are conformationally constrained to allow for a more rigid geometry between two antigen-binding domains. In some embodiments, the antibodies comprise two antigen binding domains (in the same antibody or two different antibodies) which are parallel to one another or form an angle less than a certain number of degrees upon engaging the target antigen(s). In some embodiments, the antibodies comprise two antigen binding domains (in the same antibody or two different antibodies) that are no more than a certain distance from each other upon engaging the target antigen(s). In some embodiments, the antibodies comprise two antigen binding domains (in the same antibody or two different antibodies).

In some aspects, provided herein, are antibodies designed to adopt unique interfaces between two antigen-binding domains that bind to one or more target antigens (e.g., receptor(s) that require clustering or multimerization for activation). In some embodiments, the interface between the two antigen-binding domains is adjusted so that the geometry by which the two antigen binding domains engage their target receptors similarly as how the natural ligand of the receptor(s) engages the target receptors via cluster or multimerize. For example, TNFRs become activated by ligands of the TNF superfamily. The TNFSF ligands (TNFLs) form a structurally comparatively homogeneous protein family and are characterized by a C-terminal TNF homology domain (THD), which promotes the assembly into homotrimeric and in a few cases also into heterotrimeric molecules superfamily. See e.g., Front Cell Dev Biol. 2021 Feb. 11; 8:615141. See e.g., Comput Struct Biotechnol J. 2020 Jan. 18; 18:258-270 for a computational simulation of spatial-temporal process of binding between TNF ligands and their receptors. In some embodiments, the interface between the two antigen-binding domains is adjusted so that the geometry by which the two antigen binding domains engage their target receptors comprises a compact i-shape.

In some embodiments, the angle between the two antigen-binding that bind to the same antigen or adjacent antigen(s) (e.g., receptors that clustered or multimerized) is measured via electron microscopy. See e.g., FIG. 2D. In some embodiments, the two antigen binding domains are parallel to one another. In some embodiments, the two antigen binding domains form an angle of no more than about 90 degrees (e.g., less than about 75 degrees, 70 degrees, 65 degrees, 60 degrees, 50 degrees, 45 degrees, 40 degrees, or 35 degrees) between the two domains. In some embodiments, the angle between the two antigen-binding domains is no more than about 30 degrees, no more than about 25 degrees, no more than about 20 degrees, no more than about 15 degrees, or no more than about 10 degrees. In some embodiments, the angle between the two antigen-binding domains is about 5 degrees.

In some embodiments, “i-shape” format described herein refers to antibodies that adopt a format when engaging with the antigen(s) such that two antigen binding domains (in a single antibody or two antibodies) that target the antigen(s) form an angel of no more than about 45 degrees (e.g., no more than about 40, 35, 30, 25, 20, 15, 10 or 5 degree). In some embodiments, “i-shape” format described herein refers to antibodies that adopt a format when engaging with the antigen(s) such that two antigen binding domains (in a single antibody or two antibodies) that target the antigen(s) form an angel of no more than 5 degree. In some embodiments, “i-shape” format described herein refers to antibodies that adopt a format when engaging with the antigen(s) such that two antigen binding domains (in a single antibody or two antibodies) that target the antigen(s) are parallel to one another.

In some embodiments, the two adjacent antigen-binding domains that bind to one or more target antigens (e.g., a receptor molecule requiring clustering or multimerizing for activation) have a distance (e.g., mean distance) that is about or within the range of the distance between two adjacent receptors when they cluster or multimerize upon bound by the receptor's natural ligand(s). In some embodiments, the two adjacent antigen-binding domains have a distance (e.g., mean distance) of no more than about 12 nm, 11 nm, 10 nm, 9 nm, 8 nm, 7 nm, 6 nm, 5 nm, 4 nm, 3 nm, 2 nm, or 1 nm upon engaging. In some embodiments, the target antigen is a cytokine receptor (e.g., an IL-2 receptor), and the two adjacent antigen-binding domains have a distance (e.g., mean distance) of about 3-8 nm (e.g., 3-5 nm or 5-8 nm). In some embodiments, the antibody is an IgG antibody with two Fab arms and possesses a mean paratope-paratope distance of no more than about 12 nm, 11 nm, 10 nm, 9 nm, 8 nm, 7 nm, 6 nm, 5 nm, 4 nm, 3 nm, 2 nm, or 1 nm. In some embodiments, the distance (e.g., the mean distance) between the two adjacent antigen-binding that bind to the one or more target antigen(s) (e.g., receptors that clustered or multimerized) is also measured via electron microscopy.

In some embodiments, the antibody or antibodies are IgG antibodies and adopt the constrained conformation (e.g., the i-shaped format) in dynamic equilibrium with a Y-shaped conformation exhibited by a typical IgG antibody. In some embodiments, the antibody or antibodies adopts the i-shaped confirmation in a majority particles when assessed by negative stain electron microscopy. In some embodiments, the i-shaped conformation is observed in at least about 15%, 20%, 30%, 40%, or 50% of particles. In some embodiments, the i-shaped conformation is observed in about or at least about 50% to about 60%, about 60% to about 70%, about 70% to about 80%, about 80% to about 90%, about 90% to about 100% of particles. In some embodiments, at least more than about 50% or 60% of the particles adopt the iAb conformation.

In some embodiments, the i-shape format is induced in antibodies of interest through engraftment of specific sets of mutations. In some embodiments, the i-shape format is induced in antibodies of interest through an affinity interface. In some embodiments, the affinity interface utilizes a hydrophobic patch on the surface of the heavy chain variable domain (VH domain) to facilitate intramolecular or intermolecular association between two antigen-binding domains, thereby promoting an i-shape format.

### 2. Exemplary VH Domains

In some embodiments, the antibodies described herein have one or more specific residue on the VH domain that promotes a VH-VH affinity interface or domain exchange, thereby adopt a constrained conformation (e.g., an i-shape format). See e.g., Cell. 2021 May 27; 184 (11): 2955-2972.e25; J Virol. 2010 October; 84 (20): 10700-10707. In some embodiments, the one or more specific residues promote a hydrophobic patch between two VH domains. In some embodiments, the one or more specific residues are in the framework sequence of a VH domain of a human or humanized antibody.

In some embodiments, the antibody comprises a first heavy chain variable (VH) domain and a first light chain variable (VL) domain, wherein the first VH and the first VL binds to a first target and wherein the VH domain comprises, according to Kabat numbering: one or more of 7S, 17T, 19V, 21L, 68T, 70F, 77Q, 79I, 81I, and 82aT. In some embodiments, the VH domain comprises, according to Kabat numbering: 7S, 17T, 19V, 21L, 68T, 70F, 77Q, 79I, 81I, and 82aT. In some embodiments the human or humanized antibody does not bind to HIV. In some embodiments, the antibody comprises a human consensus framework or a substantially similar framework in the VH domain (e.g., having at least 90%, 95%, 98%, 99% sequence identity) except the residues at 7, 17, 19, 21, 68, 70, 77, 79, 81, and 82.

In some embodiments, the antibody comprises a first heavy chain variable (VH) domain and a first light chain variable (VL) domain, wherein the first VH and the first VL binds to a first target and wherein the first VH domain comprises, according to Kabat numbering: one or more of 7S, 17S, 19I, 21S, 68F, 70F, 77T, 79Y, 81V, and 82aS. In some embodiments, the first VH domain comprises, according to Kabat numbering: 7S, 17S, 19I, 21S, 68F, 70F, 77T, 79Y, 81V, and 82aS. In some embodiments the human or humanized antibody does not bind to HIV. In some embodiments, the antibody comprises a human consensus framework or a substantially similar framework in the VH domain (e.g., having at least 90%, 95%, 98%, 99% sequence identity) except the residues at 7, 17, 19, 21, 68, 70, 77, 79, 81, and 82a.

In some embodiments, the antibody comprises a first heavy chain variable (VH) domain and a first light chain variable (VL) domain, wherein the first VH and the first VL binds to a first target and wherein the first VH domain comprises, according to Kabat numbering: one or more of 14A, 19I, 39R, 43G, 57R, 74L, 75E, 77F, 82aH, 82bK, 82 cM, 84V, and 113P. In some embodiments, the first VH domain comprises, according to Kabat numbering: 14A, 19I, 39R, 43G, 57R, 74L, 75E, 77F, 82aH, 82bK, 82 cM, 84V, and 113P. In some embodiments the human or humanized antibody does not bind to HIV. In some embodiments, the antibody comprises a human consensus framework or a substantially similar framework in the VH domain (e.g., having at least 90%, 95%, 98%, 99% sequence identity) except the residues at 14, 19, 39, 43, 57, 74, 75, 77, 82a, 82b, 82c, 84, and 113.

In some embodiments, the antibody (e.g., a human or humanized antibody) is derived from a reference antibody, wherein the antibody and the reference antibody both comprise a heavy chain variable (VH) domain and a light chain variable (VL) domain. In some embodiments, the VH of the antibody comprises at least one or more substitutions at the position(s) selected from 19, 21, 70, 79, and 81. In some embodiments, the VH domain comprises at least two, at least three, at least four, or at least five substitutions at the position(s) selected from 19, 21, 70, 79, and 81. In some embodiments, the VH domain comprises at least one, at least two, at least three, at least four, at least five, at least six, at least seven, at least eight, or at least nine substitutions at the position(s) selected from 7, 17, 19, 21, 68, 70, 77, 79, 81, and 82. In some embodiments, the VH domain comprises, according to Kabat numbering, at least one or more (e.g., at least two, at least three, at least four, or at least five) amino acid substitutions selected from the group consisting of 7S, 17T, 19V, 21L, 68T, 70F, 77Q, 79I, 81I, and 82aT. In some embodiments, the VH domain comprises, according to Kabat numbering: 7S, 17T, 19V, 21L, 68T, 70F, 77Q, 79I, 81I, and 82aT. In some embodiments, the amino acid substitutions are substitutions compared to the reference antibody. In some embodiments, the antibody (e.g., the human or humanized antibody) has increased agonistic activity relative to the reference antibody. In some embodiments the human or humanized antibody does not bind to HIV.

In some embodiments, the antibody (e.g., a human or humanized antibody) is derived from a reference antibody, wherein the antibody and the reference antibody both comprise a heavy chain variable (VH) domain and a light chain variable (VL) domain. In some embodiments, the VH of the antibody comprises at least one or more substitutions at the position(s) selected from 19, 68, 70, and 81. In some embodiments, the VH of the human or humanized antibody comprises at least one, at least two, at least three, or at least four substitutions at the position(s) selected from 19, 68, 70, and 81. In some embodiments, the VH domain comprises at least one, at least two, at least three, at least four, at least five, at least six, at least seven, at least eight, or at least nine substitutions at the position(s) selected from 7, 17, 19, 21, 68, 70, 77, 79, 81, and 82a. In some embodiments, the VH domain comprises, according to Kabat numbering, at least one or more (e.g., at least two, at least three, at least four, or at least five) amino acid substitutions selected from the group consisting of 7S, 17S, 19I, 21S, 68F, 70F, 77T, 79Y, 81V, and 82aS. In some embodiments, the VH domain comprises, according to Kabat numbering: 7S, 17S, 19I, 21S, 68F, 70F, 77T, 79Y, 81V, and 82aS. In some embodiments, the amino acid substitutions are substitutions compared to the reference antibody. In some embodiments, the antibody (e.g., the human or humanized antibody) has increased agonistic activity relative to the reference antibody. In some embodiments the human or humanized antibody does not bind to HIV.

In some embodiments, the antibody (e.g., a human or humanized antibody) is derived from a reference antibody, wherein the antibody and the reference antibody both comprise a heavy chain variable (VH) domain and a light chain variable (VL) domain. In some embodiments, the VH of the human or humanized antibody comprises at least one or more substitutions at the position(s) selected from 14, 19, 39, 43, 74, 77, 82a, and 82b. In some embodiments, the VH of the human or humanized antibody comprises at least one, at least two, at least three, at least four, at least five, at least six, at least seven, or at least eight substitutions at the position(s) selected from 14, 19, 39, 43, 74, 77, 82a, and 82b. In some embodiments, the VH domain comprises at least one, at least two, at least three, at least four, at least five, at least six, at least seven, at least eight, or at least nine substitutions at the position(s) selected from 14, 19, 39, 43, 57, 74, 75, 77, 82a, 82b, 82c, 84, and 113. In some embodiments, the VH domain comprises, according to Kabat numbering, at least one or more (e.g., at least two, at least three, at least four, or at least five) amino acid substitutions selected from the group consisting of 14A, 19I, 39R, 43G, 57R, 74L, 75E, 77F, 82aH, 82bK, 82 cM, 84V, and 113P. In some embodiments, the VH domain comprises, according to Kabat numbering: 14A, 19I, 39R, 43G, 57R, 74L, 75E, 77F, 82aH, 82bK, 82 cM, 84V, and 113P. In some embodiments, the amino acid substitutions are substitutions compared to the reference antibody. In some embodiments, the antibody (e.g., the human or humanized antibody) has increased agonistic activity relative to the reference antibody. In some embodiments the antibody (e.g., the human or humanized antibody) does not bind to HIV.

In some embodiments, the reference antibody comprises a human consensus framework or a substantially similar framework in the VH domain (e.g., having at least 90%, 95%, 98%, 99% sequence identity).

### 3. Exemplary Target(s) for the Antibodies

The targets for the antibodies described herein can be any molecule (e.g., a protein molecule) that activation of which (e.g., activation of its downstream signaling) is desired or a portion thereof (e.g., a subunit). In some embodiments, the activation of the molecule involves or requires clustering or multimerization of the molecule. In some embodiments, the activation of the molecule involves or requires dimerization or trimerization of the molecule. In some embodiments, the activation of the molecule involves or requires homo- or hetero-multimerization (e.g., dimerization or trimerization).

In some embodiments, the target is a cell surface receptor. In some embodiments, the cell surface receptor is a mammalian cell surface receptor (e.g., a human cell surface receptor, a non-human primate cell surface receptor, a rodent cell surface receptor, etc.). Cell surface receptors include, for example, receptors that belong to receptor families such as the hematopoietic factor receptor family, cytokine receptor family, tyrosine kinase receptor family, serine/threonine kinase receptor family, tumor necrosis factor (TNF) receptor family (TNFR), alternatively referred to as TNF receptor superfamily (TNFRSF), G protein-coupled receptor (GPCR) family, GPI-anchored receptor family, tyrosine phosphatase receptor family, adhesion factor family, and hormone receptor family. Various references that relate to receptors belonging to these receptor families and their characteristics are available and include, for example, Cooke B A., King R J B., van der Molen H J. ed. New Comprehensive Biochemistry Vol. 18B “Hormones and their Actions Part II” pp. 1-46 (1988) Elsevier Science Publishers BV., New York, USA; Patthy L. (1990) Cell, 61:13-14; Ullrich A., et al. (1990) Cell, 61:203-212; Massagul J. (1992) Cell, 69:1067-1070; Miyajima A., et al. (1992) Annu. Rev. Immunol., 10:295-331; Taga T. and Kishimoto T. (1992) FASEB J., 7:3387-3396; Fantl W I., et al. (1993) Annu. Rev. Biochem., 62:453-481; Smith C A., et al. (1994) Cell, 76:959-962; Flower D R. (1999) Biochim. Biophys. Acta, 1422:207-234; and M. Miyasaka ed., Cell Technology, supplementary volume, Handbook series, “Handbook for Adhesion Factors” (1994) (Shujunsha, Tokyo, Japan).

Cell surface receptors include, for example, hormone receptors and cytokine receptors. An exemplary hormone receptor includes, for example, estrogen receptor. Exemplary cytokine receptors include, for example, hematopoietic factor receptor, lymphokine receptor, growth factor receptor, differentiation control factor receptor and the like. Examples of cytokine receptors are erythropoietin (EPO) receptor, thrombopoietin (TPO) receptor, granulocyte colony stimulating factor (G-CSF) receptor, macrophage colony stimulating factor (M-CSF) receptor, granular macrophage colony stimulating factor (GM-CSF) receptor, tumor necrosis factor (TNF) receptor, interleukin-1 (IL-1) receptor, interleukin-2 (IL-2) receptor, interleukin-3 (IL-3) receptor, interleukin-4 (IL-4) receptor, interleukin-5 (IL-5) receptor, interleukin-6 (IL-6) receptor, interleukin-7 (IL-7) receptor, interleukin-9 (IL-9) receptor, interleukin-10 (IL-10) receptor, interleukin-11 (IL-11) receptor, interleukin-12 (IL-12) receptor, interleukin-13 (IL-13) receptor, interleukin-15 (IL-15) receptor, interferon-alpha (IFN-alpha) receptor, interferon-beta (IFN-beta) receptor, interferon-gamma (IFN-gamma) receptor, growth hormone (GH) receptor, insulin receptor, blood stem cell proliferation factor (SCF) receptor, vascular epidermal growth factor (VEGF) receptor, epidermal cell growth factor (EGF) receptor, nerve growth factor (NGF) receptor, fibroblast growth factor (FGF) receptor, platelet-derived growth factor (PDGF) receptor, transforming growth factor-beta (TGF-beta) receptor, leukocyte migration inhibitory factor (LIF) receptor, ciliary neurotrophic factor (CNTF) receptor, oncostatin M (OSM) receptor, and Notch family receptor. Additional non-limiting examples of cytokine receptors are disclosed in Wang et al. (2009) Ann. Rev. Immunol. 27:29-60.

### IL-2 Receptor

In some embodiments, the antibody (e.g., the human or humanized antibody) binds to the interleukin-2 (IL-2) receptor. The IL-2 cytokine forms a high affinity quaternary complex with 3 receptors: IL-2RA, IL-2RB, and IL2RG. IL-2RB and IL-2RG are responsible for downstream signaling upon heterodimerization, while IL-2RA stabilizes the complex and enhances IL-2 potency (See e.g., Spolski, R. et al., Nat Rev Immunol. 2018. 18, 648-659). In some embodiments, the antibody (e.g., the human or humanized antibody) binds to the IL-2 receptor comprises a bispecific antibody. In some embodiments, the antibody (e.g., the human or humanized antibody) binds to both IL-2RB, and IL2RG (such as those disclosed in the examples).

In some embodiments, the antibody (e.g., the human or humanized antibody) comprises a bispecific antibody that binds to the IL-2 receptor (e.g., via binding to both IL-2RG and IL-2RB).

In some embodiments, the antibody that binds to IL-2RG is derived from a reference antibody selected from the group consisting of G02, G12, G23, G25, G28, and G33. In some embodiments, the antibody that binds to IL-2RB is derived from a reference antibody selected from the group consisting of B09, B10, B26, B30, B37, B39, B43, and B65. In some embodiments, the antibody comprises a VH domain comprising 3 VH CDR sequences of B10. In some embodiments, the antibody comprises a VL domain comprising 3 VL CDR sequences of B10.

In some embodiments, the antibody (e.g., the human or humanized antibody) comprises a bispecific antibody that binds to the IL-2 receptor comprising a first Fab and a second Fab, wherein the first Fab is derived from a reference antibody selected from the group consisting of G02, G12, G23, G25, G28, and G33, and the second Fab is derived from a reference antibody selected from the group consisting of B09, B10, B26, B30, B37, B39, B43, and B65. In some embodiments, the antibody (e.g., the human or humanized antibody) comprises a bispecific antibody that binds to the IL-2 receptor, wherein one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28.

### Tumor Necrosis Factor Receptor Superfamily (TNFRSF)

In certain embodiments, the target is a member of the tumor necrosis factor receptor (TNFR) family. TNFRSF receptors are normally activated by molecular clustering induced by cognate cell membrane tumor necrosis factor superfamily (TNFSF) ligands. In some embodiments, the TNFRSF member target to trigger its downstream signaling via clustering. In some embodiments, the TNFRSF member target shares a similar ligand-receptor trimeric structure for signaling activation. In some embodiments, the antibodies (e.g., the human or humanized antibodies) adopting the iAb conformation can induce cell membrane receptor clustering and activation of TNFRSF members. In some embodiments, the formation of an active TNFSF₃-TNFRSF₃ complex is the minimal unit for TNFRSF signaling activation. In some embodiments, a TNFRSF member may require oligomerization of two or more TNFSF₃-TNFRSF₃ complexes to be able to fully stimulate TNFRSF downstream signaling cascades. Non-limiting examples of TNFRs include TNFR1, TNFR2, lymphotoxin β receptor, OX40, CD40, Fas, decoy receptor 3, CD27, CD70, CD226, CD137, ICOS, 2B4, CD30, 4-1BB, death receptor 3 (DR3), death receptor 4 (DR4), death receptor 5 (DR5), death receptor 6 (DR6), decoy receptor 1, decoy receptor 2, receptor activator of NF-kappa B (RANK), osteoprotegerin (OPG), TWEAK receptor, TACI, BAFF receptor (BAFF-R), HVEM (herpes virus entry mediator, nerve growth factor receptor, B cell maturation antigen (BCMA), glucocorticoid-induced TNF receptor (GITR), toxicity and JNK inducer (TAJ), RELT, TNFRSF22, TNFRSF23, ectodysplasin A2 isoform receptor and ectodysplasin 1, anhidrotic receptor. Additional non-limiting examples of TNFRs are disclosed in Naismith and Sprang (1998) Trends in Biochemical Sciences 23 (2): 74-79. In some embodiments, the antibody (e.g., the human or humanized antibody) binds to OX40, CD40, 4-1BB, DR4, or DR5.

In some embodiments, the antibody binds to a cysteine-rich domain (CRD) subunit of the TNFR. In some embodiments, the antibody binds to a cysteine-rich domain (CRD) subunit outside of the ligand-binding domain (i.e., the domain bound by a natural ligand that binds to the TNFR). In some embodiments, the antibody binds to a membrane distal CRD domain of the TNFR.

In some embodiments, the antibody (e.g., the human or humanized antibody) binds to OX40 (e.g., a human OX40). OX40 is also known as tumor necrosis factor receptor superfamily, member 4 (TNFRSF4), or CD134. OX40 is predominantly expressed on effector and regulatory T-cells. The natural ligand of OX40, OX40L, is expressed on activated antigen-presenting cells, including dendritic cells, endothelial cells, macrophages, and activated B-cells. In some embodiments, OX40-OX40L engagement is key to potentiating the expansion of effector T-cells and the prolongation of their survival by suppressing apoptosis, enhancing T-cell effector functions, such as cytokine production, and generating T helper memory cells. See e.g., Pharmaceutics. 2022; 14 (12): 2753.

In a particular embodiment, a human or humanized antibody that binds to OX40 is modified to adopt an iAb conformation using the methods described herein. In some embodiments, the antibody (e.g., the human or humanized antibody) that binds to OX40 is derived from an OX40 antibody selected from the group consisting of 3C8, 1A7, 2A3, 2B5, 2F10, 2G7, 2H5, 3F5, 3G5, and 3G8. See e.g., Biomolecules. 2022 September; 12 (9): 1209, Clin Cancer Res. 2018 Nov. 15; 24 (22): 5735-5743, Proc Natl Acad Sci USA. 2022 Jun. 7; 119 (23): e2201562119.

In some embodiments, the OX40 antibody is derived from a reference antibody that is selected from HFB301001, FS120, INBRX-106, BGB-A445, PF-04518600, MEDI6469, MEDI0562, ABBV-368, FS120, INCAGN01949, BMS986178, PF04518600, GSK3174998, and SL-279252. See e.g., Curr Oncol Rep. 2022 July; 24 (7): 951-960, Cancer Immunol Res 2020; 8:781-93; Journal for ImmunoTherapy of Cancer 2020; 8: doi: 10.1136/jitc-2020-SITC2020.0699.

In some embodiments, the antibody (e.g., the human or humanized antibody) binds to CD40 (e.g., a human CD40). CD40 is also known as tumor necrosis factor receptor superfamily, member 5 (TNFRSF5). CD40 is expressed on platelets, B cells, and myeloid cells, but also on non-hematopoietic cells like endothelial cells, fibroblasts, smooth muscle cells and certain types of tumor cells. The cognate ligand for CD40 is CD154 (TNFSF₅/CD40L). CD40 expression on monocytes, and their progeny macrophages and dendritic cells (DCs), and B cells plays an important role in immune cell function. CD40 signaling is an important trigger of the monocyte maturation process and mainly drives differentiation into macrophages of the M1 spectrum and DCs.

In some embodiments, the CD40 antibody is derived from a reference antibody that is selected from CP-870,893 (RO70099789), SGN-40, selicrelumab, dacetuzumab, Chi Lob 7/4, APX005M, ADC-1013, CDX-1140, SEA-CD40, ravagalimab, giloralimab, and sotigolimab. See e.g., Oncol Lett. 2020 November; 20 (5): 176.

In some embodiments, the antibody (e.g., the human or humanized antibody) binds to 4-1BB (e.g., a human 4-1BB). 4-1BB is also known as CD137 and TNFRSF9. 4-1BB is expressed on both CD4+ and CD8+ T cells, as well as natural killer (NK) cells and DCs. 4-1BB provides co-stimulatory signals and activates cytotoxic effects of CD8+ T cells and helps to form memory T cells. In addition, 4-1BB signaling can activate NK cells and dendritic cells. Like other TNFRSF members, three monomeric 4-1BBs bind to a trimeric CD137L to activate intracellular signaling.

In some embodiments, the 4-1BB antibody is derived from a reference antibody that is selected from urelumab (BMS-663513), EU 101, sytalizumab, LVGN-6051, YH-004, GEN1046, PRS343, ES101, Cinrebafusp alfa, HLX-35, IBI309, TJ-033721, ATG 101, LBL-024, MCLA-145, ABL-503, PM 1032, QLF-31907, FS-120, RO7227166, HBM-7008, ND-021, GNC-035, GNC-038, GNC-039, ADG-106, utomilumab, ATOR-1017, AGEN-2373, CTX-471, PRS-344, RO-7122290 and HOT-1030. See e.g., Front Immunol. 2022 Sep. 16; 13:975926.

In some embodiments, the antibody (e.g., the human or humanized antibody) binds to DR4 (e.g., a human DR4). DR4 is also known as TRAIL receptor 1 (TRAILR1) and tumor necrosis factor receptor superfamily member 10A (TNFRSF10A). In some embodiments, the antibody (e.g., the human or humanized antibody) binds to DR5 (e.g., a human DR5). DR5 is also known as TRAIL receptor 2 (TRAILR2) and tumor necrosis factor receptor superfamily member 10B (TNFRSF10B). DR4 and DR5 are cell surface receptors of the TNF-receptor superfamily that bind tumor necrosis factor-related apoptosis inducing ligand (TRAIL), and mediate apoptosis. Engagement of DR4 or DR5 with TRAIL triggers apoptosis through recruitment of the adapter protein Fas-associated death domain (FADD) and the formation of the macromolecular complex called death-inducing signaling complex (DISC).

In some embodiments, the DR4 antibody is derived from a reference antibody that is selected from HLX56, mapatumumab, m921/922, 4H6, 4G7, AY4, and TR1-mAbs. See e.g., Antibodies (Basel). 2017 December; 6 (4): 16.

In some embodiments, the DR5 antibody is derived from a reference antibody that is selected from conatumumab, drozitumab, DS-8273a, KTRM2, lexatumumab, tigatuzumab, zaptuzumab, inbrx-109, LaDR5, LBy135, mDRA6, WD1, zaptuximab, HMCAZ5, and AD5.10. See e.g., Antibodies (Basel). 2017 December; 6 (4): 16.

In some embodiments, the target is a member of the low-density lipoprotein receptor (LDLR) family. Non-limiting examples of LDLRs include LDLR, Low-density lipoprotein receptor-related protein (LRP) 1, LRP10, LRPIB, LRP2, LRP4, LRP5, LRP5L, LRP6, LRP8, Nidogen (NID)-1, NID2, Sortilin-related receptor, L (SORL1) and Very-low-density-lipoprotein receptor (VLDLR).

In some embodiments, the target is a member of the receptor tyrosine kinases (RTK) family. Non-limiting examples of RTKs include Leukocyte receptor tyrosine kinase (LTK), Receptor tyrosine kinase-like orphan receptors (RORs), Ephrin receptors (Ephs), Trk receptor, insulin receptor (IR) and Tie2. Additional non-limiting examples of RTKs are disclosed in Alexander et al. (2013) The Concise Guide to Pharmacology 2013/14: Enzymes. Br. J. Pharmacol. 170:1797-1867; Li and Hristova (2010); and Lemmon and Schlessinger (2010) Cell 141 (7): 1117-1134.

In some embodiments, the target is a growth hormone receptor, an insulin receptor, a leptin receptor, a Flt-3 ligand receptor, or an insulin-like growth factor (IGF)-I receptor. Exemplary receptors include, for example, hEPOR (Simon, S. et al. (1990) Blood 76, 31-3); mEPOR (D'Andrea, A D. et al. (1989) Cell 57, 277-285); hG-CSFR (Fukunaga, R. et al. (1990) Proc. Natl. Acad. Sci. USA. 87, 8702-8706); mG-CSFR (Fukunaga, R. et al. (1990) Cell 61, 341-350); hTPOR (Vigon, I. et al. (1992) 89, 5640-5644); mTPOR (Skoda, R C. et al. (1993) 12, 2645-2653); hInsR (Ullrich, A. et al. (1985) Nature 313, 756-761); hFlt-3 (Small, D. et al. (1994) Proc. Natl. Acad. Sci. USA. 91, 459-463); hPDGFR (Gronwald, R G K. et al. (1988) Proc. Natl. Acad. Sci. USA. 85, 3435-3439); hIFNa/b R (Uze, G. et al. (1990) Cell 60, 225-234; and Novick, D. et al. (1994) Cell 77, 391-400).

In some embodiments, the target is a nerve growth factor receptor family and/or the neurotrophin receptor family. Non-limiting examples of nerve growth factor receptors and neurotrophin receptors include p75 (also referred to as low affinity nerve growth factor receptor (LNGFR)), TrkA, TrkB and TrkC. Additional non-limiting examples of nerve growth factor receptors and neurotrophin receptors are disclosed in Lotz et al. (1996) J. of Leukocyte Biology 60 (1): 1-7.

In some embodiments, the target is a member of the growth factor receptor family. For example, and not by way of limitation, a growth factor receptor can be a receptor that signals through the JAK/STAT, MAP kinase and PI3 kinase pathways. Non-limiting examples of growth factor receptors include fibroblast growth factor receptors (FGFRs), ErbB family of receptors (e.g., epidermal growth factor receptor (EGFR)), vascular endothelial growth factor receptors (VEGFR) and Platelet-derived growth factor receptors (PDGFRs).

In some embodiments, the target can include receptors that form heterodimers or heterotrimers to induce a cell signal. For example, and not by way of limitation, the target can be a member of the serine/threonine kinase receptor family. Non-limiting examples of serine/threonine kinase receptors include activin A receptor type II-like I (ALK1), activin A receptor, type I (ALK2), bone morphogenetic protein receptor, type IA (BMPRIA), activin A receptor, type IB (ALK4), activin A receptor, type IC (ALK7), transforming growth factor, beta receptor 1 (TGFBR1), bone morphogenetic protein receptor, type IB (BMPR1B), transforming growth factor, beta receptor II (TGFBR2), bone morphogenetic protein receptor, type II (BMPR2), anti-Mullerian hormone receptor, type II (MISR2), activin A receptor, type IIA (ActR2), activin A receptor, type IIB (ActR2B) and transforming growth factor, beta receptor III (TGFBR3).

In some embodiments, the antibody (e.g., the human or humanized antibody) binds to the receptor of a cytokine, wherein the cytokine can form a complex in nature with at least two distinct receptors, which triggers downstream activity of the cytokine. In some embodiments, the downstream activity of the cytokine results in the activation of signal transduction pathways, gene expression changes, cell proliferation and differentiation, cell survival, cell death, metabolic changes, or cytoskeletal rearrangement. In some embodiments, the downstream activity of the cytokine activates a signal transduction pathway such as the Akt, AMPK, apoptosis, estrogen, insulin, JAK-STAT, MAPK, mTOR, NF-κB, Notch, p53, TGF-β, Toll-like, VEGF, or Wnt signaling pathway.

### 4. Antibody Affinity

In certain aspects, an antibody described herein (e.g., the antibody provided by the present application, e.g., the reference antibody) has a dissociation constant (K_{D}) of ≤1 μM, ≤ 100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M).

In certain aspects, an antibody described herein (e.g., the antibody provided by the present application, e.g., the reference antibody) has a dissociation constant (KD) between 100 nM and 1 μM, between 10 nM and 100 nM, between 1 nM and 10 nM, between 0.1 nM and 1 nM, between 0.01 nM and 0.1 nM, or between 0.001 and 0.1 nM.

In certain aspects, an antibody described herein (e.g., the antibody provided by the present application, e.g., the reference antibody) has one or more modifications that promotes a weaker dissociation constant against the target.

In certain aspects, an antibody described herein (e.g., the antibody provided by the present application, e.g., the reference antibody) has an off-rate constant (k_{off}) of ≤10-2 s⁻¹, ≤ 5λ10-3 s⁻¹, ≤10-3 s⁻¹, ≤5×10-4 s⁻¹, ≤10-4 s⁻¹, ≤5×10-5 s⁻¹, or ≤10-5 s⁻¹. In certain aspects, an antibody provided herein has an off-rate (k_{off}) between 10-2 s⁻¹ and 5×10-3 s⁻¹, between 10-3 s⁻¹ and 5×10-3 s⁻¹, between 5×10-4 s⁻¹ and 10-3 s⁻¹, between 10-4 s⁻¹ and 5×10-4 s⁻¹, between 10-4 s⁻¹ and 5×10-4 s⁻¹, between 5×10-5 s⁻¹ and 10-4 s⁻¹ or between 10-5 s⁻¹ and 5×10-5 s⁻¹.

In certain aspects, an antibody described herein (e.g., the antibody provided by the present application, e.g., the reference antibody) has one or more modifications that promotes a quicker off-rate constant against the target.

In one aspect, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D})) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In an alternative method, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed, and the plate washed eight times with 0.1% polysorbate 20 (TWEEN-20®) in PBS. When the plates have dried, 150 μl/well of scintillant (MICROSCINT-20 ™, Packard) is added, and the plates are counted on a TOPCOUNT™ gamma counter (Packard) for ten minutes. Concentrations of each Fab that give less than or equal to 20% of maximal binding are chosen for use in competitive binding assays.

### 5. Antibody Fragments

In certain aspects, the antibody described herein (e.g., the antibody provided by the present application, e.g., the reference antibody) is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 6. Chimeric and Humanized Antibodies

In certain aspects, an antibody described herein (e.g., the antibody provided by the present application, e.g., the reference antibody) is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 7. Human Antibodies

In certain aspects, an antibody described herein (e.g., the antibody provided by the present application, e.g., the reference antibody) is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 8. Multispecific Antibodies

In certain aspects, an antibody described herein (e.g., the antibody provided by the present application, e.g., the reference antibody) is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for a target and the other specificity is for any other antigen. In certain aspects, bispecific antibodies may bind to two (or more) different epitopes of a target. Multispecific (e.g., bispecific) antibodies may also be used to localize cytotoxic agents or cells to cells which express a target. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to a target as well as another different antigen, or two different epitopes of a target (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for a target and the other is for a different target (e.g., another antigen).

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) e1203498.

In some embodiments, the multiple specific or bispecific antibody bind to at least two antigens that are two subunits of a molecule, wherein the multimerization of the two subunits promotes the activation of the molecule.

### 9. Antibody Variants

Amino acid sequence variants of an antibody described herein may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### (i) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or decreased or increased effector functions.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated, and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### (ii) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the application may be made in order to create antibody variants with certain improved properties.

In one aspect, antibody variants are provided having a non-fucosylated oligosaccharide, i.e., an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

(iii) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In some embodiments, the antibody described herein comprises one or more amino acid substitutions that allows for a decreased effector function. In some embodiments, the antibody comprises an IgG Fc with one or more substitutions selected from L234A, L235A, P329G. In some embodiments, the antibody comprises L234A, L235A and P329G.

In some embodiments, the antibody comprises one or more mutations that promote multimerization of the antibodies. In some embodiments, the antibody comprises an IgG Fc comprising E345R, E430G, and/or S440Y.

In some embodiments, the antibody has a modified Fc that promotes its binding to FcgRIIB. In some embodiments, the antibody has an IgG Fc comprising S267E. In some embodiments, the antibody has an IgG Fc comprising S267E and L328F.

In certain aspects, the application contemplates an antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96R non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (e.g., diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g., Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

In certain aspects, an antibody variant comprises an Fc region with one or more amino acid substitutions which decrease self-recognition, e.g., substitutions at positions R355, E356, K414, E438, K439, S440 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In some aspects, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### (iv) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. No. 7,521,541, 8,30,930, 7,855,275, 9,000,130, or WO 2016040856.

### (v) Hinge Modification

In certain aspects, the antibody provided herein comprises a modified hinge (e.g., as compared to the reference antibody). In some embodiments, the antibody comprises an IgG2 hinge or substantially similar hinge. In some embodiments, the antibody comprises an IgG4 hinge or substantially similar hinge. In some embodiments, the antibody comprises an IgG1 hinge or substantially similar hinge. In some embodiments, the antibody comprises an IgG3 hinge or substantially similar hinge. Structural analysis revealed that the IgG2 isotype had the most rigid hinge region wherein the least active isotype (IgG3) was the most flexible. See e.g., Trends Mol Med. 2023 January; 29 (1): 48-60.

In some embodiments, the antibody does not comprise an IgG3 hinge.

In some embodiments, the modified hinge confers more restricted hinge flexibility. In some embodiments, the modified hinge restricts hinge flexibility by at least 10% compared to a corresponding reference antibody. In some embodiments, the modified hinge restricts hinge flexibility by between about 10% and about 30%, about 30% and about 50%, about 50% and about 70%, about 70% and about 90%, or about 90% to about 100% compared to a corresponding reference antibody. In some embodiments, the modified hinge restricts hinge flexibility by 50% compared to a corresponding reference antibody. Flexibility can be assessed using e.g., computational models. See e.g., J Pharm Sci. 2019 May; 108 (5): 1663-1674.

### 10. Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Recombinant Methods and Compositions

The antibodies described herein (e.g., antibodies provided by the present application, e.g., reference antibodies) may be produced using recombinant methods and compositions, e.g., as described in the examples. For these methods one or more isolated nucleic acid(s) encoding an antibody are provided. When more than one isolated nucleic acids were used, these nucleic acids can be on the same expression vector or on different expression vectors, normally these nucleic acids are located on two or three expression vectors, i.e., one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as reported herein are provided.

In some embodiments, provided herein are nucleic acid(s) encoding the antibodies (e.g., human or humanized antibodies). In some embodiments, the nucleic acid encodes a human or humanized antibody that adopts an iAb conformation. In some embodiments, the nucleic acid encodes a human or humanized antibody that adopts an iAb conformation through a domain exchange mechanism. In some embodiments, the nucleic acid encodes a human or humanized antibody that adopts an iAb conformation through an affinity interface mechanism.

In some embodiments, the nucleic acid provided herein are in one or more vectors. For example, in some embodiments, provided herein is a vector comprising a nucleic acid encoding a human or humanized antibody. In some embodiments, the vector comprises the nucleic acid(s) encoding a human or humanized antibody of the present disclosure.

In some embodiments, the vector is a viral vector. In some embodiments, the vector is a retroviral vector. In some embodiments, the vector is a gamma retroviral vector. In some embodiments, the vector is a lentiviral vector. In some embodiments, the vector is an adenoviral vector. In some embodiments, the vector is an adeno-associated viral (AAV) vector. In some embodiments, the vector is a pEF-ENTR A vector.

In some embodiments, the vector encodes multiple gene products. In some embodiments, the vector is a bicistronic vector. In some embodiments, the vector comprises a nucleic acid that encodes a second protein product, e.g., a fluorescent protein such as green fluorescent protein (GFP).

In some embodiments, the vector is a transposase vector. In some embodiments, the vector is a piggyBac vector.

In some embodiments, the vector comprises a promoter. In some embodiments, the nucleic acid encoding the human or humanized antibody is operably linked to the promoter. In some embodiments, the promoter is an inducible promoter. In some embodiments, the promoter is a ubiquitously expressed promoter. In some embodiments, the vector comprises an EF1-a promoter. In some embodiments, the nucleic acid encoding the chimeric receptor is operably linked to the EF1-a promoter.

In one aspect, a method of making an antibody described herein is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of agonist antibodies and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as YO, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., YO, NSO, Sp20 cell).

### C. Assays

The antibodies provided herein may be identified, screened for, or characterized for their physical/chemical properties and/or biological activities by various assays known in the art.

### Agonist Activity and Affinity

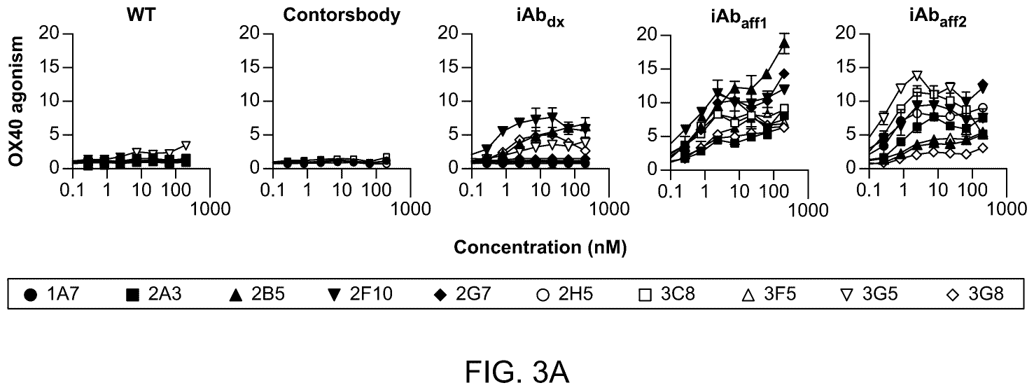

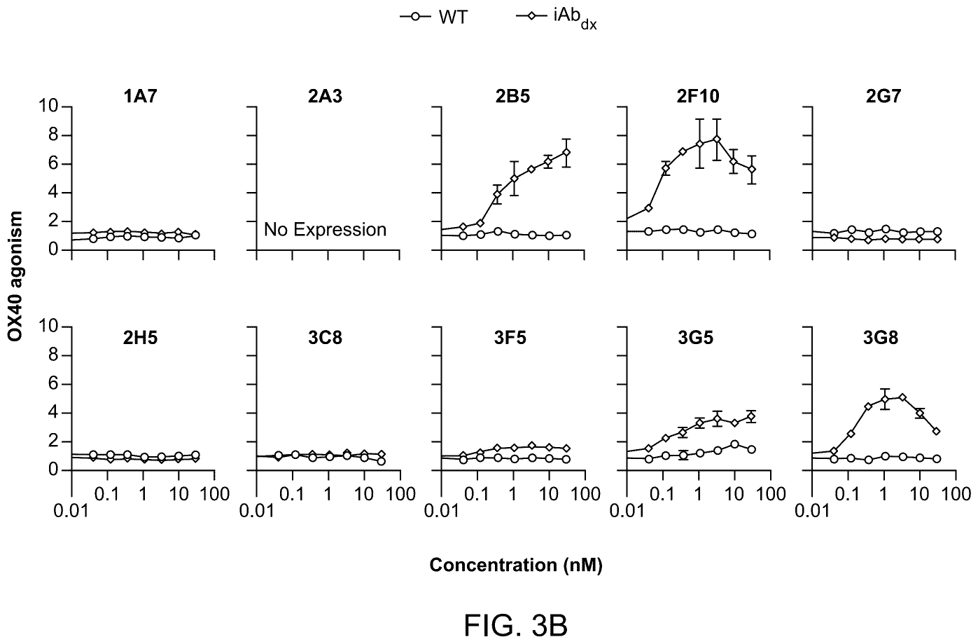

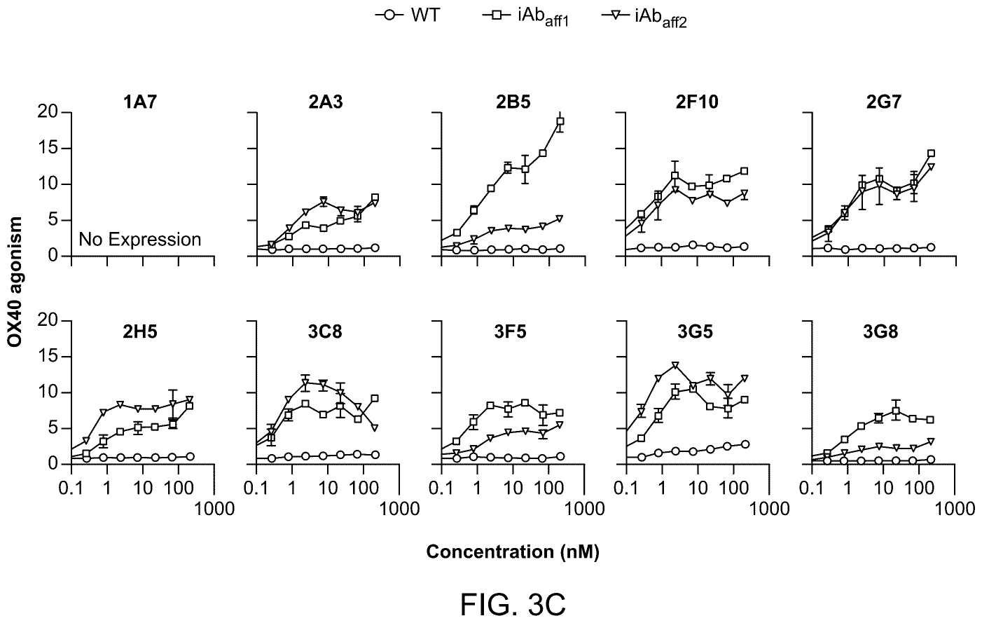

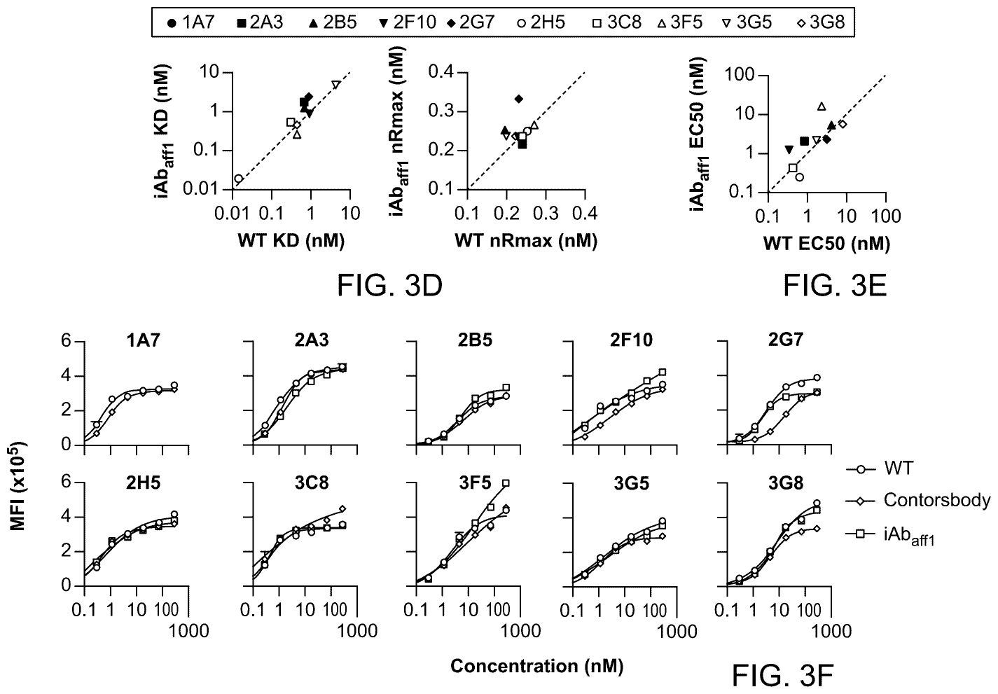

In certain aspects, the antibody (e.g., the human or humanized antibody) described herein is tested for its agonistic activity. In some embodiments, the agonistic activity is relative to an untreated control cell or a cell exposed to a control antibody (e.g., the reference antibody discussed herein). In some embodiments, the relative agonistic activity of the human or humanized antibody towards a target receptor is tested in a cell-based assay utilizing a human cell line expressing a target receptor and a luciferase reporter system. For example, see FIG. 3A. In some embodiments, agonistic activity is evaluated by fold change of the reporter expression signal over a control (e.g., an untreated control). In some embodiments, values greater than at least 2-fold indicate target receptor agonism. In some embodiments, agonism towards a target receptor results in an increase of at least about 2-fold, 5-fold, 10-fold, 15-old, or 20-fold expression of a reporter gene, e.g. luciferase. In some embodiments, corresponding wild-type (WT) IgG antibodies or contorsbodies are tested as comparators. In some embodiments, human or humanized antibodies adopting an iAb format demonstrate improved agonistic activity over a WT IgG control antibody and/or a contorsbody.

In some embodiments, the antibody (e.g., the human or humanized antibody) activates a target via receptor clustering. In some embodiments, the agonistic activity of the human or humanized antibody results in an increase in the proportion of activated receptors on a target cell. In some embodiments, the proportion of activated receptors on a target cell increases at least 1-fold, at least 1.5-fold, at least 2-fold, at least 2.5-fold, or at least 3-fold compared to an untreated target cell. In some embodiments, the proportion of activated receptors on a target cell increases 2-fold compared to an untreated target cell.

### Binding Assays and Other Assays for Antibodies in the Agonist Antibodies

In one aspect, an antibody is tested for its antigen binding activity, e.g., by known methods such as ELISA, Western blot, etc.

In some embodiments, the binding of the human or humanized antibody to a target receptor described herein is tested by surface plasmon resonance (SPR) to measure solution-phase 1:1 affinity. In some embodiments, the binding of the human or humanized antibody to a target receptor described herein is tested by other known methods such as FACS-based cell binding, ELISA, Western blot, etc. In some embodiments, human or humanized antibodies adopting an iAb format possess similar K_{D} and normalized Rₘₐₓ (nRₘₐₓ) values to corresponding WT IgG antibodies. nRₘₐₓ values represent the theoretical normalized max response for analyte binding. In some embodiments, the iAb conformation does not alter the K_{D} and nRₘₐₓ values of human or humanized antibodies compared to corresponding WT IgG antibodies. In some embodiments, the iAb conformation does not alter the avidity of human or humanized antibodies to their target receptor compared to corresponding WT IgG antibodies. In some embodiments, human or humanized antibodies adopting an iAb format possess similar EC50 and maximum signal values to corresponding WT IgG antibodies. In some embodiments, the antibodies (e.g., the human or humanized antibodies) possess an EC50 value that differs by at most about 5% to about 20% to that of a corresponding WT IgG antibody. In some embodiments, the antibodies (e.g., the human or humanized antibodies) possess an EC50 value that differs by at most about 20% to that of a corresponding WT IgG antibody. In some embodiments, the antibodies (e.g., the human or humanized antibodies) possess a maximum signal value that differs by at most about 5% to about 20% to that of a corresponding WT IgG antibody. In some embodiments, the antibodies (e.g., the human or humanized antibodies) possess a maximum signal value that differs by at most about 20% to that of a corresponding WT IgG antibody.

In another aspect, competition assays may be used to identify an antibody that competes with a reference antibody for binding to a desired target. In certain aspects, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized target is incubated in a solution comprising a first labeled antibody that binds to target (e.g., reference antibody) and a second unlabeled antibody that is being tested for its ability to compete with the first antibody for binding to the target. The second antibody may be present in a hybridoma supernatant. As a control, immobilized target is incubated in a solution comprising the first labeled antibody but not the second unlabeled antibody. After incubation under conditions permissive for binding of the first antibody to target, excess unbound antibody is removed, and the amount of label associated with immobilized target is measured. If the amount of label associated with immobilized target is substantially reduced in the test sample relative to the control sample, then that indicates that the second antibody is competing with the first antibody for binding to target. See Harlow and Lane (1988) Antibodies: A Laboratory Manual ch.14 (Cold Spring Harbor Laboratory, Cold Spring Harbor, NY).

### Activity Assays

In one aspect, assays are provided for identifying human or humanized antibodies having biological activity. Biological activity may include, e.g., binding to a target substrate. Antibodies having such biological activity in vivo and/or in vitro are also provided.

In certain aspects, an antibody described herein is tested for such biological activity.

### D. Libraries and Generation of Libraries

In certain aspects, the present application further provides libraries (e.g., libraries comprising the VH and VL domains of the antibodies described herein) and uses thereof.

In some embodiments, there is provided a library comprising polynucleotides, wherein the polynucleotides in the library encode at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering: a) 7S, 17T, 19V, 21L, 68T, 70F, 77Q, 79I, 81I, and 82aT; b) 7S, 17S, 19I, 21S, 68F, 70F, 77T, 79Y, 81V, and 82aS; or c) 14A, 19I, 39R, 43G, 57R, 74L, 75E, 77F, 82aH, 82bK, 82 cM, 84V, and 113P.

In some embodiments, there is provided a library comprising at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering: a) 7S, 17T, 19V, 21L, 68T, 70F, 77Q, 79I, 81I, and 82aT; b) 7S, 17S, 19I, 21S, 68F, 70F, 77T, 79Y, 81V, and 82aS; or c) 14A, 19I, 39R, 43G, 57R, 74L, 75E, 77F, 82aH, 82bK, 82 cM, 84V, and 113P.

In some embodiments, the antibodies are expressed or to be expressed on the surface of one or more phages or yeast cells.

Antibodies of the application may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12:725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

Certain aspects of the present disclosure relate to libraries for screening antibodies adopting a constrained conformation (e.g., an iAb conformation) for agonistic activity. In some embodiments, the libraries comprise a plurality of polynucleotides each encoding an antibody. In some embodiments, the libraries comprise a plurality of antibodies. In some embodiments, the antibodies in the library are displayed on cell surfaces and phage surfaces. In some embodiments, the antibody comprises an antibody heavy chain variable domain (VH) and/or an antibody light chain variable domain (VL). The libraries described herein are useful for screening for and/or identifying one or more agonistic antibodies.

In some embodiments, the S. cerevisiae yeast displayed scFv library containing a diversity of at least 1.6×10⁹ unique sequences is used to select for binders to target proteins. In some embodiments, the S. cerevisiae yeast displayed scFv library is used to select for binders to cell surface receptors. In some embodiments, the S. cerevisiae yeast displayed scFv library is used to select for binders to IL-2RG and IL-2RB. In some embodiments, yeast is electroporated with plasmid encoding the scFv library and grown to log phase at 30° C. in SDCAA media. In some embodiments, yeast representing 10× diversity of the library were grown at 20° C. in SGCAA media containing galactose for 24-48 hours at an OD600 of 1.0 prior to each round of selection. In some embodiments, for initial rounds of selection using magnetic SA beads, biotinylated antigen was mixed with the beads prior to addition of yeast to enhance avidity. In some embodiments, for later rounds of selection with tetrameric SA, yeast was first stained with biotinylated antigen, washed with PBS containing 1% BSA and then stained with SA. In some embodiments, each round was checked for enrichment of a binding population by staining yeast with a titration of antigen and analyzing fluorescence.

In some embodiments of the libraries described herein, each antibody comprises a scFv comprising a heavy chain variable region (VH) and a light chain variable region (VL). In some embodiments of the libraries described herein, each antibody comprises a Fab comprising a heavy chain variable region (VH) and a light chain variable region (VL).

In some embodiments, a library of the present disclosure includes one or more vectors (e.g., an expression vector and/or display vector) comprising one or more polynucleotides (e.g., synthetic polynucleotides) of the present disclosure. In some embodiments, each antibody is fused with all or a portion of a protein (e.g., a viral coat protein, a bacterial surface protein, a yeast surface protein, an insect cell surface protein, a mammalian cell surface protein) (i.e., creating an agonist antibody). In some embodiments, the agonist antibody is displayed on the surface of a particle or a host cell. In some embodiments, a library of the present disclosure includes host cells and particles (e.g., phages) displaying the antibodies of the present disclosure.

Further provided herein is a method of preparing a library, e.g., by providing and assembling the polynucleotide sequences (e.g., synthetic polynucleotide(s)) of a library of the present disclosure. Polynucleotides encoding antibodies as described herein can be cloned into any suitable vector for expression of a portion or the entire polypeptide sequence. In some embodiments, the polynucleotide is cloned into a vector allowing for production of a portion or the entire polypeptide fused to all or a portion of a protein e.g., a viral coat protein, a bacterial surface protein, a yeast surface protein, an insect cell surface protein, a mammalian cell surface protein) (i.e., creating an agonist antibody) and displayed on the surface of a particle or cell. Several types of vectors are available and may be used to practice the present disclosure, for example, phagemid vectors. Phagemid vectors generally contain a variety of components including promoters, signal sequences, phenotypic selection genes, origin of replication sites, and other necessary components as are known to those of ordinary skill in the art. In some embodiments, the polynucleotides encoding the polypeptide regions can be cloned into vectors for expression in bacterial cells for bacterial display or in yeast cells for yeast display. Exemplary vectors are described in US PG Pub. No. US20160145604. In some embodiments, the vector is a display vector comprising, from 5′ to 3′, a polynucleotide encoding an amino acid sequence to be displayed on a surface (e.g., a surface of phage, bacteria, yeast, insect, or mammalian cells), a restriction site, a second polynucleotide encoding a surface peptide capable of being displayed on the surface, and a second restriction site. In some embodiments, the second polynucleotide encodes a phage coat protein, a yeast outer wall protein (such as Aga2), a bacterial outer membrane protein, a cell surface tether domain, or an adapter, or a truncation or derivative thereof. In some embodiments, the surface peptide is for phage display, yeast display, bacterial display, insect display, or mammalian display, or shuttling display there between. In some embodiments, when expressed, the amino acid sequence and the surface peptide are displayed as an agonist antibody on the surface. In some embodiments, the vector further comprises a fusion tag 5′ to the first restriction site or 3′ to the second restriction site.

Certain aspects of the present disclosure relate to a population of cells containing vector(s) described herein. Antibodies encoded by polynucleotides generated by any of the techniques described herein, or other suitable techniques, can be expressed and screened to identify antibodies having desired structure and/or activity. Expression of the proteins can be carried out, for example, using cell-free extracts (e.g., ribosome display), phage display, prokaryotic cells (e.g., bacterial display), or eukaryotic cells (e.g., yeast display). In some embodiments, the cells are bacterial cells, yeast cells, insect cells, or mammalian cells (such as Chinese Hamster Ovary (CHO) cells). Methods for transfecting bacterial cells, yeast cells, or mammalian cells are known in the art and described in the references cited herein. Expression (e.g., from a library of the present disclosure) of proteins in these cell types, as well as screening for antibodies of interest, are described in more detail below.

Alternatively, the polynucleotides can be expressed in an E. coli expression system, such as that described by Pluckthun and Skerra. (Meth. Enzymol., 1989, 178:476; Biotechnology, 1991, 9:273). The mutant proteins can be expressed for secretion in the medium and/or in the cytoplasm of the bacteria, as described by Better and Horwitz, Meth. Enzymol., 1989, 178:476. In some embodiments, the polypeptides are attached to the 3′ end of a sequence encoding a signal sequence, such as the ompA, phoA or pelB signal sequence (Lei et al., J. Bacteriol., 1987, 169:4379). These gene fusions are assembled in a dicistronic construct, so that they can be expressed from a single vector and secreted into the periplasmic space of E. coli where they will refold and can be recovered in active form. (Skerra et al., Biotechnology, 1991, 9:273).

In other embodiments, the polypeptide sequences of the present disclosure are expressed on the membrane surface of a prokaryote, e.g., E. coli, using a secretion signal and lipidation moiety as described, e.g., in US20040072740; US20030100023; and US20030036092.

Alternatively, polypeptide sequences of the present disclosure can be expressed and screened by anchored periplasmic expression (APEx 2-hybrid surface display), as described, for example, in Jeong et al., PNAS, 2007, 104:8247 or by other anchoring methods as described, for example, in Mazor et al., Nature Biotechnology, 2007, 25:563.

Higher eukaryotic cells, such as mammalian cells, for example myeloma cells (e.g., NS/0 cells), hybridoma cells, Chinese hamster ovary (CHO) cells, and human embryonic kidney (HEK) cells, can also be used for expression of the polypeptides of the present disclosure. Polypeptides (e.g., agonistic antibodies) expressed in mammalian cells may be designed to be secreted into the culture medium or expressed on the surface of the cell.

In other embodiments, polypeptides or antibodies (e.g., agonistic antibodies) can be selected using mammalian cell display (Ho et al., PNAS, 2006, 103:9637). In some embodiments, as described above and exemplified below, polypeptides or antibodies can be selected after production of a portion or the entire polypeptide or antibody fused to all or a portion of a viral coat protein (i.e., creating an agonist antibody) and displayed on the surface of a particle or cell, e.g., using phage display.

In some embodiments, the present application provides polynucleotides libraries encoding one or more antibodies or antibody libraries and each antibody in the library comprises an antigen binding domain. In some embodiments, the antigen binding domain comprises an antibody light chain variable region and/or an antibody heavy chain variable region. In some embodiments, the antigen binding domain comprises an antibody light chain variable region and an antibody heavy chain variable region. In some embodiments, the antigen binding domain comprises an antibody heavy chain variable region and does not comprise an antibody light chain variable region. In some embodiments, an antigen binding domain of the present disclosure comprises an antibody light chain variable region and/or an antibody heavy chain variable region with specificity for any target of interest, including any of the targets described herein.

In some embodiments, the antibody comprises a full-length antibody light chain and/or a full-length antibody heavy chain. The antibody light chain may be a kappa or lambda light chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. An antibody heavy chain described herein may be converted from one class or subclass to another class or subclass using methods known in the art.

In some embodiments, the antibodies in the library comprise full-length antibody heavy and light chains. In some embodiments, the antibodies in the library comprise antibody fragments (e.g., Fab fragments, F(ab′)2 fragments, etc.). In some embodiments, at least one of the antibodies or antibody fragments in the library binds to a target with an equilibrium dissociation constant (Kd) of about 10-7 M or less, 10-8 M or less, 10-9 M or less, 10-10 M or less, or 10-11 M or less.

In some embodiments, the library comprises at least two antibodies. In some embodiments, the library comprises at least two, at least three, at least four, at least five, at least six, at least seven, at least eight, at least nine, at least 10, at least 100, at least 1000, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ antibodies. In some embodiments, the library comprises about two, about three, about four, about five, about six, about seven, about eight, about nine, about 10, about 100, about 1000, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, or about 10²⁰ antibodies.

In some embodiments, the antibodies in the library comprise at least two unique antibody heavy chain variable regions. In some embodiments, the antibodies in the library comprise at least two, at least three, at least four, at least five, at least six, at least seven, at least eight, at least nine, at least 10, at least 100, at least 1000, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ unique antibody heavy chain variable regions. In some embodiments, the antibodies in the library comprise about two, about three, about four, about five, about six, about seven, about eight, about nine, about 10, about 100, about 1000, about 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ unique antibody heavy chain variable regions.

In some embodiments, the antibodies in the library comprise at least two unique antibody heavy chain variable regions. In some embodiments, the antibodies in the library comprise at least two, at least three, at least four, at least five, at least six, at least seven, at least eight, at least nine, at least 10, at least 100, at least 1000, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ unique antibody heavy chain variable regions. In some embodiments, the antibodies in the library comprise about two, about three, about four, about five, about six, about seven, about eight, about nine, about 10, about 100, about 1000, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, or about 10²⁰ unique antibody heavy chain variable regions.

In some embodiments, the antibodies in the library comprise at least two unique antibody heavy chain variable regions. In some embodiments, the antibodies in the library comprise at least two, at least three, at least four, at least five, at least six, at least seven, at least eight, at least nine, at least 10, at least 100, at least 1000, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ unique antibody heavy chain variable regions. In some embodiments, the antibodies in the library comprise about two, about three, about four, about five, about six, about seven, about eight, about nine, about 10, about 100, about 1000, about 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, at least 10¹⁸, at least 10¹⁹, or at least 10²⁰ unique antibody heavy chain variable regions.

### F. Methods of Screening Agonist Antibodies

In some aspects, provided herein are methods of screening polynucleotide and polypeptide libraries described herein for the desired antibodies (e.g., agonist antibodies targeting a specific antigen). The screening of antibodies derived from any of the libraries described herein may be carried out by any appropriate method known in the art, including, for example, using an ELISA, using surface plasmon resonance, using affinity chromatography, using an activity assay, etc.

In some embodiments, there is provided a method of screening an agonist antibody comprising contacting the antibodies in the any of the libraries discussed above with the target or with a cell expressing the target. In some embodiments, the method further comprises assessing agonistic activity of the antibodies.

In some embodiments, provided herein is a method of using a library of polynucleotides described herein for an agonist antibody that binds to a target, the method comprising: a) contacting an expressed antibody of the library with the target to determine a first binding affinity; b) isolating one or more of the expression products that bind to the target. In some embodiments, the method further comprises c) determining whether the one or more isolated expression products function as an agonist.

In some embodiments, provided herein is a method of using a library comprising antibodies described herein for an agonist antibody that binds to a target, the method comprising: a) contacting an expressed antibody of the library with the target to determine a first binding affinity; b) isolating one or more of the expression products that bind to the target. In some embodiments, the method further comprises c) determining whether the one or more isolated expression products function as an agonist.

In some embodiments that may be combined with any of the preceding embodiments, the target is a mammalian cell surface receptor protein. In some embodiments, the mammalian cell surface receptor protein is a tumor necrosis factor receptor (TNFR) superfamily member or a G-protein coupled receptor superfamily member. In some embodiments that may be combined with any of the preceding embodiments, the target is selected from the group consisting of OX40, GITR, CD27, CD40, CD137, DR5, 4-1BB and Tie2. In some embodiments that may be combined with any of the preceding embodiments, the target is a human protein, a non-human primate protein, or a rodent protein.

### G. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the agonist antibodies provided herein and a pharmaceutically acceptable carrier. In another aspect, a pharmaceutical composition comprises any of the agonist antibodies provided herein and at least one additional therapeutic agent, e.g., as described below.

Pharmaceutical compositions (formulations) of an agonist antibody as described herein can be prepared by combining the agonist antibodies with pharmaceutically acceptable carriers or excipients known to the skilled person. See, for example, WO2019/224842, Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1st Ed., Woodhead Publishing (2015), § 4 and Falconer R. J., Biotechnology Advances (2019), 37, 107412. Exemplary pharmaceutical compositions of an agonist antibody as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### H. Therapeutic Methods and Routes of Administration

Any of the antibodies provided herein may be used in therapeutic methods.

In one aspect, an antibody (e.g., an agonist antibody) for use as a medicament is provided. In further aspects, an antibody (e.g., an agonist antibody) for use in treating a disease or condition is provided. In certain aspects, an antibody (e.g., an agonist antibody) for use in a method of treatment is provided. In certain aspects, the application provides an antibody (e.g., an agonist antibody) for use in a method of treating an individual having a disease or condition (e.g., a disease or condition that involves an antigen) comprising administering to the individual an effective amount of the antibody. In one such aspect, the method further comprises administering to the individual an effective amount of at least one additional therapeutic agent (e.g., one, two, three, four, five, or six additional therapeutic agents), e.g., as described below. In further aspects, the application provides an antibody for use in e.g., promoting receptor clustering and activation of downstream signaling pathways of a cell surface receptor. In certain aspects, the application provides an antibody for use in a method of promoting an iAb conformation in the antibody, thereby facilitating activation of cell surface receptors in an individual. An “individual” according to any of the above aspects is preferably a human.

In a further aspect, the application provides for the use of antibodies (e.g., agonist antibodies) in the manufacture or preparation of a medicament. In one aspect, the medicament is for treatment of a disease or condition that involves or is caused by abnormal cell surface receptor signaling. In a further aspect, the medicament is for use in a method of treating a disease comprising administering to an individual having a disease an effective amount of the medicament. In one such aspect, the method further comprises administering to the individual an effective amount of at least one additional therapeutic agent, e.g., as described below. In a further aspect, the medicament is for use in a method of promoting receptor clustering and activation of downstream signaling pathways of a cell surface receptor comprising administering to the individual an effective amount of the medicament. An “individual” according to any of the above aspects may be a human.

In a further aspect, the application provides a method for treating a disease or condition (e.g., a cancer or tumor). In one aspect, the method comprises administering to an individual having such disease or condition an effective amount of an antibody (e.g., an agonist antibody) described herein.

In some embodiments, there is provided a method for treating a disease or condition (e.g., a cancer or tumor) comprising administering two or more distinct antibodies (e.g., two Fabs, e.g., two scFvs), wherein the two distinct antibodies bind to two distinct antigens. In some embodiments, the two distinct antigens are two subunits of a molecule that requires or involves clustering or multimerization of the two subunits for activation of the downstream signaling. In some embodiments, the two distinct antigens are two molecules that are involved in a complex (e.g., a complex on a cell surface, e.g., T cell receptor complex) or a portion thereof, the formation of which confers activation of a signaling pathway. In some embodiments, the two distinct antigens are two members of TNSFSR.

In some embodiments, there is provided a method for treating a disease or condition (e.g., a cancer or tumor) comprising administering a multispecific antibody (e.g., a bispecific antibody), wherein the antibody bind to two distinct antigens. In some embodiments, the two distinct antigens are two subunits of a molecule that requires or involves clustering or multimerization of the two subunits for activation of the downstream signaling. In some embodiments, the two distinct antigens are two molecules that are involved in a complex (e.g., a complex on a cell surface, e.g., T cell receptor complex), the formation of which confers activation of a signaling pathway. In some embodiments, the two distinct antigens are two members of TNSFSR.

In some embodiments, the method further comprises administering to the individual an effective amount of at least one additional therapeutic agent, as described below.

An “individual” according to any of the above aspects may be a human.

In a further aspect, the application provides a method for promoting agonistic activity of the human or humanized antibody against the antigen and/or facilitating receptor clustering and activation of downstream signaling pathways in the cells of an individual. In one aspect, the method comprises administering to the individual an effective amount of an antibody. In one aspect, an “individual” is a human.

In a further aspect, the application provides pharmaceutical compositions comprising any of the antibodies provided herein, e.g., for use in any of the above therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Antibodies of the application can be administered alone or used in a combination therapy. For instance, the combination therapy includes administering an antibody of the application and administering at least one additional therapeutic agent (e.g., one, two, three, four, five, or six additional therapeutic agents). In certain aspects, the combination therapy comprises administering an antibody of the application and administering at least one additional therapeutic agent.

Such combination therapies noted above encompass combined administration (where two or more therapeutic agents are included in the same or separate pharmaceutical compositions), and separate administration, in which case, administration of the antibody of the application can occur prior to, simultaneously, and/or following, administration of the additional therapeutic agent or agents. In one aspect, administration of the antibody and administration of an additional therapeutic agent occur within about one month, or within about one, two or three weeks, or within about one, two, three, four, five, or six days, of each other. In one aspect, the antibody and additional therapeutic agent are administered to the patient on Day 1 of the treatment. Antibodies of the application can also be used in combination with radiation therapy.

An antibody of the application (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The antibodies of the application would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the application (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. For repeated administrations over several days or longer, depending on the condition, the treatment would generally be sustained until a desired suppression of disease symptoms occurs. However, other dosage regimens may be useful. The progress of this therapy is easily monitored by conventional techniques and assays.

### IV. ARTICLES OF MANUFACTURE

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the application may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following examples are included for illustrative purposes only and are not intended to limit the scope of the present disclosure.

### Example 1: Structural Determinants of Previously Described i-Shaped Antibodies (iAbs)

Recent studies have characterized a subset of broadly neutralizing HIV antibodies isolated from infected humans and rhesus macaques that share a unique linear i-shaped conformation that is distinct from the conventional Y-shape. These antibodies have a decreased paratope-paratope distance driven by intramolecular association between Fab domains. Physiologically, this close proximity simultaneously increases the avidity of the interaction with viral surface glycans and generates additional paratopes at the Fab-Fab interface.

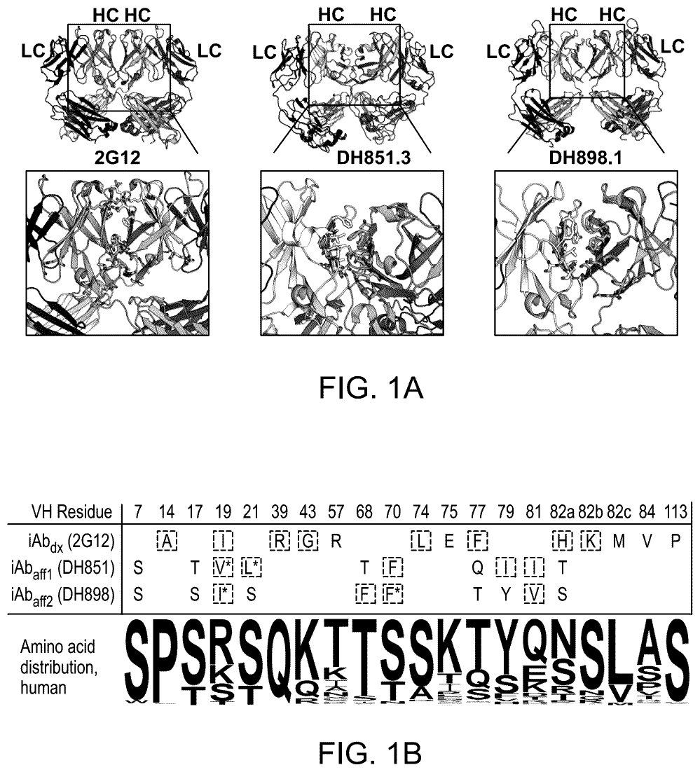

Thorough studies of these i-shaped antibodies (iAbs) revealed distinct, independently evolved mechanisms that determine this unique conformation. One human antibody isolated from an HIV patient, referred to as 2G12, achieves its iAb conformation through heavy chain variable (VH) domain exchange between Fabs (FIG. 1A, left) (Calarese, D. A., et al., Science, 2003. 300, 2065-2071 and Trkola, A., et al., J Virol., 1996. 70, 1100-1108). In SHIV-infected rhesus macaques, two antibody lineages were identified, referred to as DH851 and DH898, with an affinity-driven intramolecular Fab-Fab homotypic interaction between VH domain β-strands A, B, D, and E (FIG. 1A, middle and right) (Williams, W. B., et al., Cell, 2021. 184, 2955-2972.e25). Both of these mechanisms involve non-covalent Fab-Fab association mediated through distinct yet topologically similar inter-VH interfaces (FIG. 1).

Consistent with structural studies, specific residues contributing to domain exchange are located exclusively in the VH domain and have little to no impact on antigen binding. A set of VH residues was selected from 2G12, referred to as iAb_{dx}, that were hypothesized to be most critical for inducing the domain-exchanged iAb conformation (FIG. 1B). Similarly, a set of residues were designed based on structural analyses and predicted to mediate the Fab-Fab affinity interaction in the DH851 and DH898 lineages and were termed iAb_{aff1} and iAb_{aff2}, respectively (FIG. 1B). Several of the amino acids found at these residues were significantly underrepresented with regard to known antibody sequences (FIG. 1B) and are not germline encoded, suggesting that they arose in the repertoire from somatic mutation.

### Example 2: Engineered Residue Grafts Induce iAb Conformation

Because antibodies share a high degree of sequence and structural homology, it was hypothesized that mutating the sites identified above within a given antibody of interest could be sufficient to induce iAb formation. To test this hypothesis, each putative iAb-inducing residue set (iAb_{dx}, iAbafr1, and iAb_{aff2}) was grafted into a panel of 10 distinct anti-OX40 antibody clones with diverse sequences, germline precursors, epitopes, and affinities (FIGS. 2A-C) (Leonard, B., et al., Proc National Acad Sci, 2022. 119, e2201562119). OX40 is a therapeutically relevant TNFRSF member, for which receptor clustering is known to play a role in activation. As a comparator, all 10 anti-OX40 antibodies were also produced as contorsbodies, which is a recently described conformationally constrained format that uses genetic linkers to fuse the heavy and light chains of the Fabs to the N- and C-termini of the Fc domain, respectively (Georges, G. J., et al., Comput Struct Biotechnology J., 2020. 18, 1210-1220).

The impact of the engrafted iAb residues on antibody conformation was assessed with negative stain electron microscopy. 2D classes from representative anti-OX40 antibody clones with each residue set were compared to those of wildtype (WT) IgG and a representative contorsbody clone (FIG. 2D). Each iAb yielded 2D classes clearly showing two Fabs interacting in parallel and resembling previously reported 2D classes of 2G12 and the macaque broadly neutralizing HIV antibodies (Wu, Y., et al., Cell Reports, 2013. 5, 1443-1455). The images of the iAb_{dx} clone contained a distribution of conformations with 29% of the particles as i-shaped antibodies and the remaining 71% as standard Y-shaped antibodies. For both the iAb_{aff1} and iAb_{aff2} residue sets, approximately 64% of the particles adopted the iAb conformation, while the standard Y-shaped IgG conformation was observed for the remaining particles. In addition, iAb dimer conformations, in which the Fabs associate in an intermolecular head-to-head manner, were observed in the iAb_{aff2} images (FIG. 2D, far right image). This result is consistent with size exclusion chromatography results (FIGS. 2E and 2F). Concentration-dependent studies of monomer: dimer ratios support the noncovalent nature of the affinity-based iAb interfaces and suggest the iAb interaction is in dynamic equilibrium (FIG. 2G). As expected, none of the wildtype IgG images were found to have the unique iAb shape, and the contorsbody adopted the barrel-like conformation with the Fabs pinned next to the Fc (FIG. 2D).

### Example 3: iAb Reformatting Enables Intrinsic OX40 Agonist Activity

Like many TNFRSF members, standard antibodies against OX40 generally do not intrinsically promote signaling, but rather rely on extrinsic crosslinking to drive receptor clustering via Fc engagement with cell surface Fc gamma receptors (FcgRs), crosslinking using secondary antibodies, or coating on beads or plates. To determine whether iAb formation could enhance the activity of the engrafted anti-OX40 antibodies, their activity was tested in a cell-based assay utilizing a Jurkat cell line engineered to express OX40 and a nuclear factor κB (NF-κB) luciferase reporter. The corresponding WT IgGs and contorsbodies were tested as comparators. Antibody clones expressed as monospecific WT IgG or contorsbody formats had little to no activity (FIG. 2H). Conversely, OX40 agonism activity was observed across all iAb formats with the affinity interfaces, iAb_{aff1} and iAb_{aff2}, showing the strongest and most consistent gain of function (FIGS. 2H and 2I-2J). Only 4/9 iAb_{dx} engrafted clones had intrinsic agonism activity (clone 2A3 did not express), while all iAb_{aff1} and iAb_{aff2} engrafted clones demonstrated some improvement in activity over the WT IgG control. Given the variable activity of the iAb_{dx} panel and the dimer contaminant observed in iAb_{aff2} samples (FIGS. 2D-G), the iAb_{aff1} graft was utilized for the remainder of the study.

To determine if the enhanced activity of the engineered iAbs was due to impacts in antigen binding, surface plasmon resonance (SPR) was performed to measure solution-phase 1:1 affinity. WT IgG and iAb_{aff1} engrafted clones had both similar K_{D} and normalized Rₘₐₓ (nRₘₐₓ) values across the entire anti-OX40 panel tested, indicating that the iAb_{aff1} residue set does not affect affinity or the ability of both antibody Fabs to bind OX40 simultaneously (FIG. 2K). In addition to this solution-based analysis, FACS-based cell binding experiments were also performed to determine whether avidity was affected by the iAb conformation upon antigen recognition on the cell surface. WT IgGs and iAbs for each anti-OX40 clone had similar cell binding in terms of both EC50 and maximum signal (FIGS. 2L and 2M). Collectively these data show that the intrinsic agonism activity of the iAb format is not a consequence of altered affinity or cell surface avidity.

### Example 4: The iAb Interface is Capable of Driving OX40 Agonism as an Intermolecular Interaction

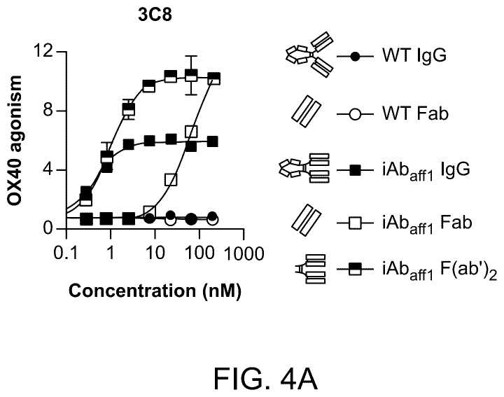

To further characterize the iAb interface, an experiment to probe the strength of the homotypic Fab interaction was performed. Because a binding measurement can be confounded by intramolecular Fab interaction in the context of a bivalent IgG, the iAb_{aff1} engrafted 3C8 anti-OX40 clone was expressed as a monomeric Fab and tested whether the iAb interface could enable agonist activity in a monovalent format through intermolecular interaction (FIG. 4A). The iAb interface still enabled intrinsic agonist activity as a monomeric Fab, albeit at an EC50 reduced nearly 100-fold compared to iAb IgG. Strikingly, the maximal activity was increased almost 2-fold for the iAb Fab relative to iAb IgG. Intrigued by this result, a F(ab′)₂ version of the 3C8 antibody was produced and a similarly heightened level of maximal activity that had an EC50 equivalent to iAb IgG was observed (FIG. 4A). These results suggest that the presence of the Fc region in a full-length IgG may sterically restrict iAb activity. These results indicate that the iAb conformation can enable intrinsic agonism through both intra and intermolecular Fab interactions.

### Example 5: iAbs Broadly Enable Intrinsic TNFRSF Agonism

After demonstrating the ability of the iAb conformation to intrinsically agonize OX40, additional studies to explore whether the same constrained conformation could be generalized to enable agonism against other TNFRSF members were conducted. Four panels of publicly available and in-house derived antibodies were produced with diverse sequences, germline precursors, and affinities against CD40, 4-1BB, DR4, and DR5 as both WT IgG and iAb_{aff1} formats. A variant (C131S) IgG2 was produced that promotes the constrained h2B isoform as a comparator for CD40.

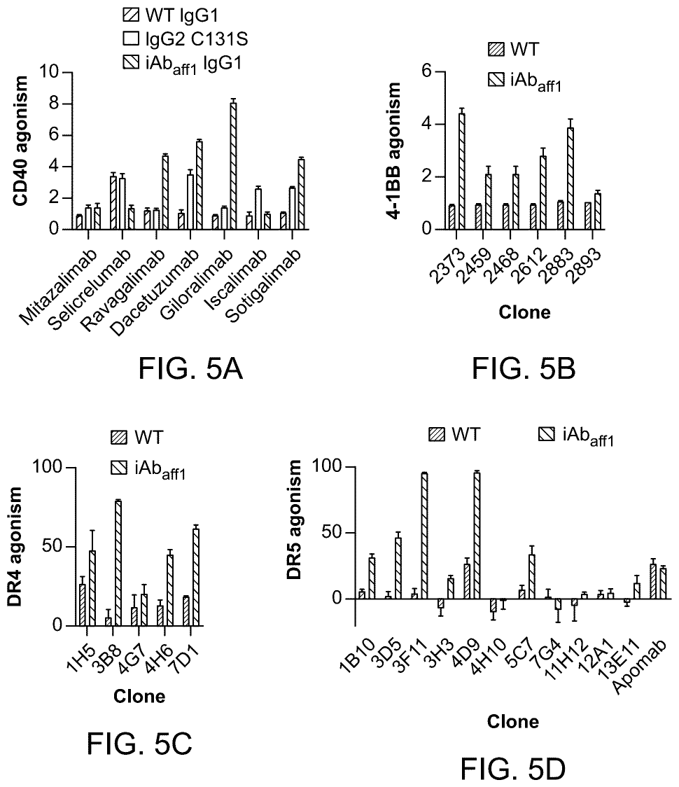

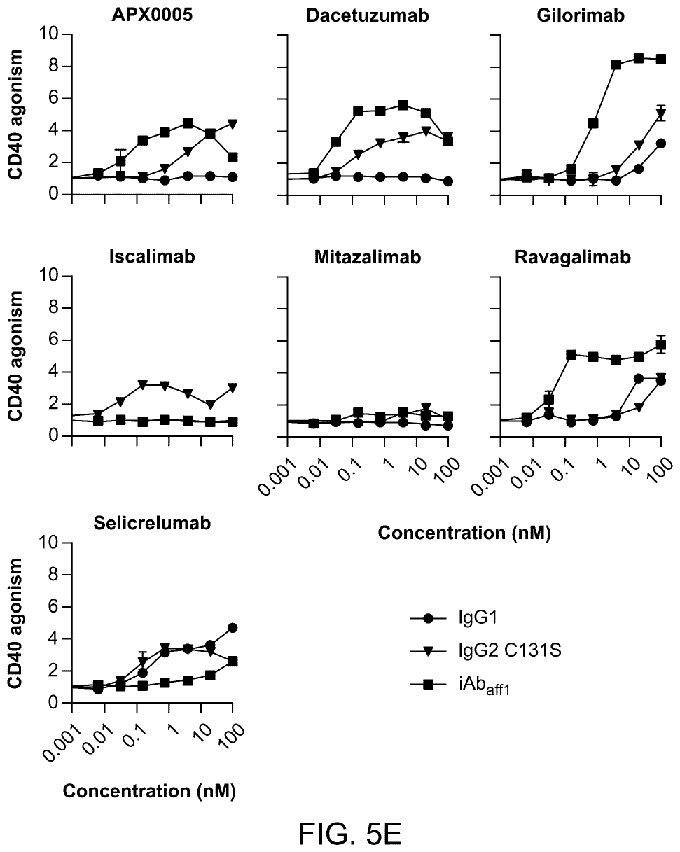

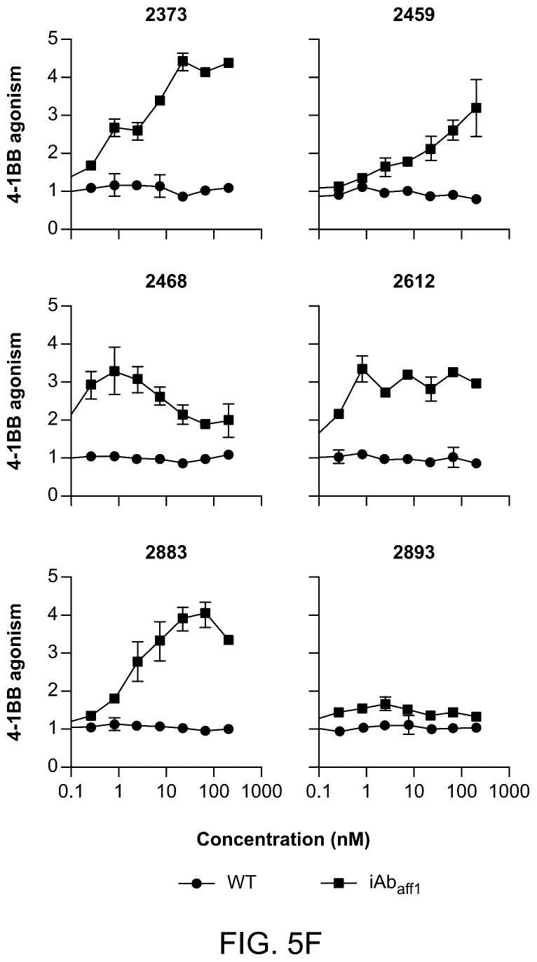

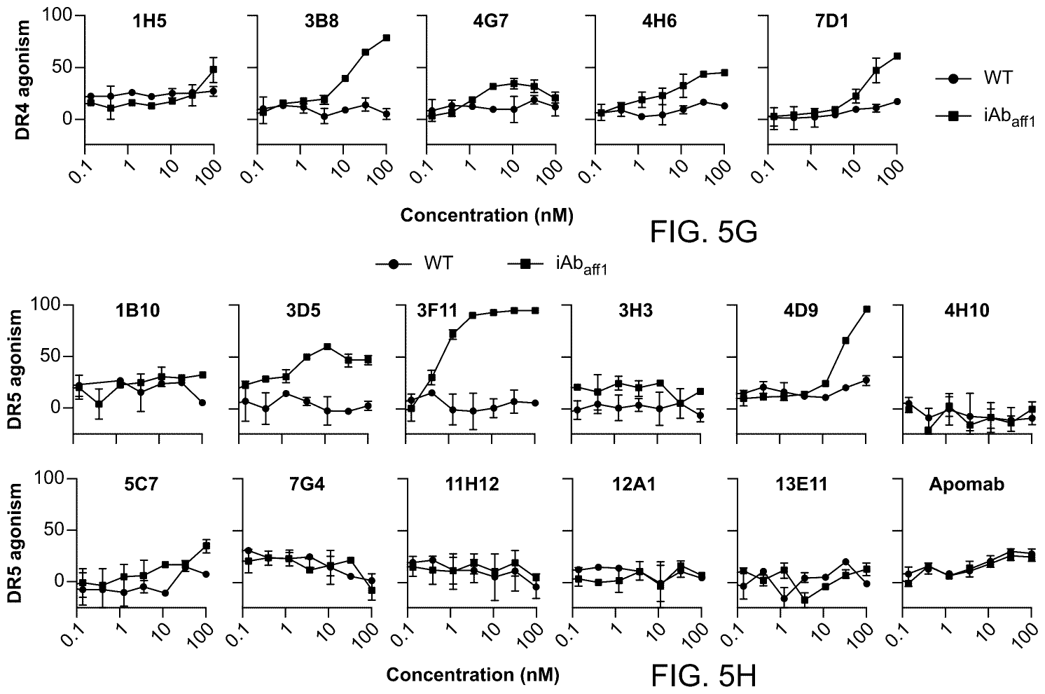

For a majority of the clones across all targets, iAb reformatting enhanced agonist activity compared to the corresponding WT IgG format, which generally showed low or no agonism (FIGS. 5A-5H). For CD40, there was no apparent correlation between a given antibody's activity in the different formats (WT IgG, IgG2 C131S, iAbafr1) and its previously reported epitope (Smith, K. E., et al., Expert Opin Biol Th, 2021. 21, 1635-1646). However, the strongest agonism activity was observed for iAb_{aff1} formats of ravagalimab, dacetuzumab, giloralimab, and sotigolimab (FIG. 5A). The most consistent results were observed for 4-1BB agonism, where 5/6 iAb clones enabled intrinsic activity at least 2-fold greater than untreated control, in contrast to the corresponding WT IgG comparators that were all inactive without crosslinking (FIG. 5B). This result may suggest more relaxed epitope and/or Fab binding orientation constraints governing agonism of this receptor. DR4 and DR5 agonism proved to be more variable with only 3/5 anti-DR4 iAb clones showing a 2-fold increase in activity over WT IgG controls and 5/12 anti-DR5 iAbs able to kill at least 25% of cells (FIGS. 5C and 5D). Overall, these results highlight the broad applicability of the constrained iAb format as an engineering tool to generate effective intrinsic agonists against TNFRSF members.

### Example 6: Comparison Between Bivalent iAbs and Highly Avid Hexameric IgG

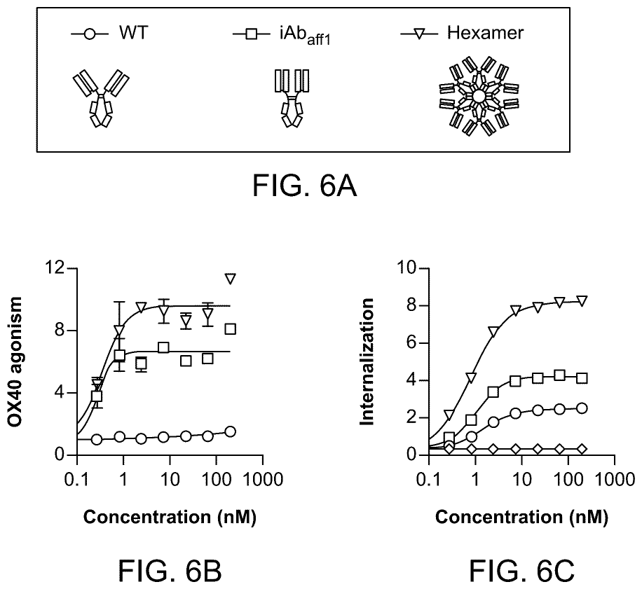

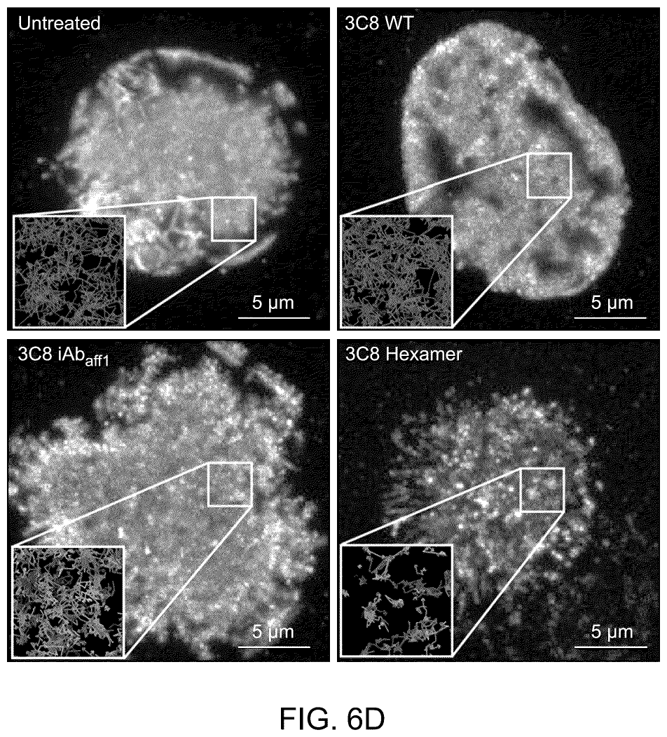

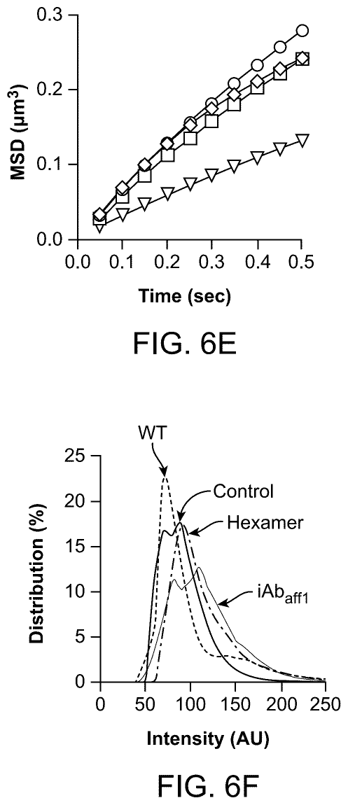

Activation of many TNFRSF members has been shown to benefit from higher order receptor clustering mediated by extrinsic crosslinking. Because of this, engineering approaches to increase avidity, such as IgG hexamerization, have generated effective antibody-based agonists. Given the enhanced activity of the iAb over WT IgG in the absence of a discernable difference in binding properties, the conformation-based mechanism of action was compared to that of avidity. To do so, mutations were introduced in the Fc of the 3C8 antibody known to induce hexamerization and drive intrinsic OX40 agonism (FIG. 6A). In the OX40 NF-κB reporter assay, the 3C8 iAb had a similar EC50 with slightly lower maximum signal compared to the hexameric IgG format (FIG. 6B), which is striking given that hexameric 3C8 has a valency of 12, while the 3C8 iAb only has a valency of 2. In addition to activity, the effect of these formats on receptor-mediated internalization was assessed by labeling the antibodies with a pH-sensitive dye that increases in fluorescence under low pH conditions such as those present in acidified lysosomes.

Using flow cytometry to detect and quantify fluorescence, the hexameric IgG was found to drive substantially increased internalization relative to WT IgG, concomitant with its high intrinsic activity (FIG. 6C). Interestingly, while the iAb similarly mediates strong intrinsic OX40 agonism, albeit slightly less than hexameric IgG, the associated level of receptor downregulation is modestly greater than the inactive WT IgG. These results offer the prospect that different engineering approaches to intrinsic agonism can have different activity versus internalization profiles, the consideration of which would have clear importance for biotherapeutic design.

The mechanistic impacts of the 3C8 iAb and hexamer were further explored using total internal reflection fluorescence (TIRF) microscopy to track clustering patterns and single particle mobility of fluorescently tagged OX40 following treatment with the antibody formats. Max projections for the entire 12.5 second acquisition under each treatment condition showed a largely diffuse distribution of OX40 for both the untreated and WT 3C8 treated samples, whereas the iAb and hexamer treated samples had hotspots of receptor accumulation with the hexamer having a more punctate pattern (FIG. 6D). Inset windows with the molecular trajectories illustrate how each molecule moves and provide additional clarity on individual molecular confinement. Mean square displacement analyses of the trajectories further highlighted the differences between the formats and clearly showed that the hexamer restricts movement of OX40 compared to the other formats (FIG. 6E). While the iAb conformation still allowed more free 2D diffusive movement of OX40 similar to untreated cells and WT 3C8 (FIG. 6E), closer analysis of the individual track intensity values showed a shift in the distribution for the iAb similar to that of the hexamer (FIG. 6F). Taken together, these data indicate a propensity for both the iAb and hexamer to tightly link two or more receptors, explaining the increased activity and internalization driven by these two formats. Yet, there may be mechanistic discrepancies, as the hexamer induces higher order, larger scale clusters of OX40 with greatly reduced mobility within the membrane, potentially explaining the greater activity and more pronounced internalization caused by treatment with this format.

### Example 7: Discovery of Antibodies to IL-2RG and IL-2RB for Application of iAb Engineering to Cytokine Mimetics

Given the success in applying iAbs to monospecific agonists across multiple TNFRSF targets, additional studies were conducted to test whether the platform could be applied to bispecific antibody agonists of a cytokine receptor. The IL-2 pathway was chosen for assessment due to the significant interest in ligand mimetic agonists in the field. The IL-2 cytokine forms a high affinity quaternary complex with 3 receptors: IL-2RA, IL-2RB, and IL2RG. IL-2RB and IL-2RG are responsible for downstream signaling upon heterodimerization, while IL-2RA stabilizes the complex and enhances IL-2 potency.

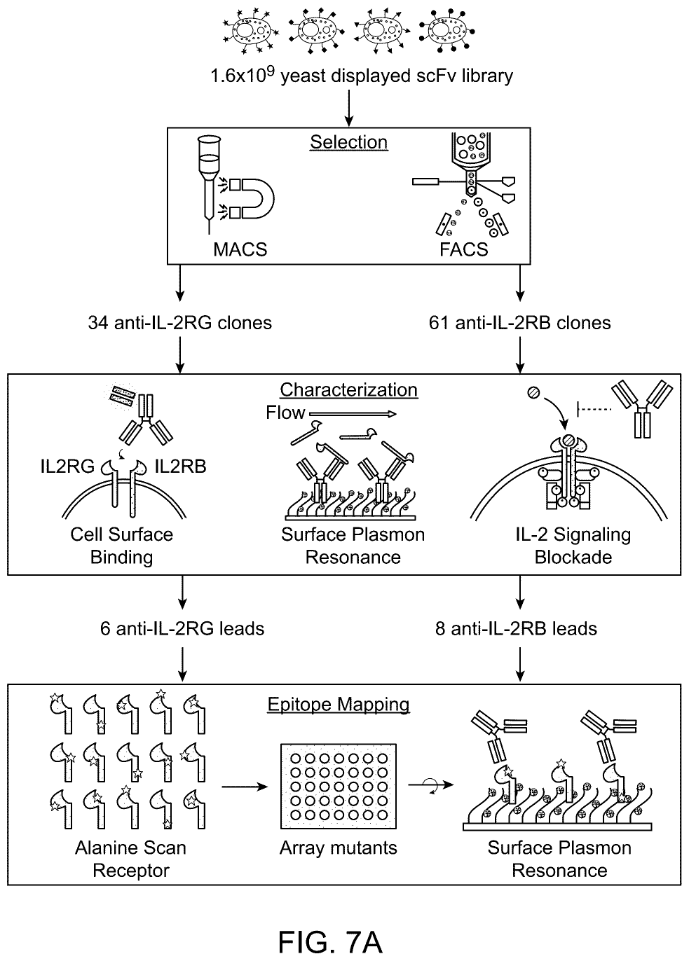

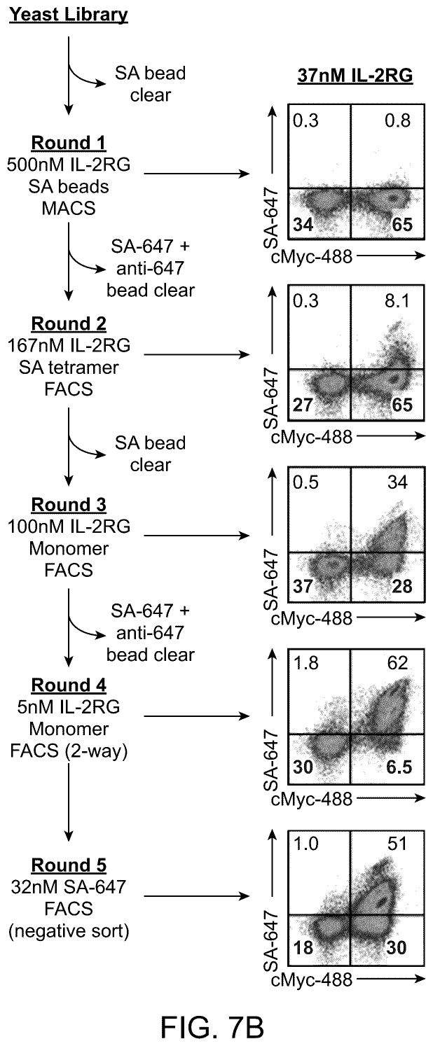

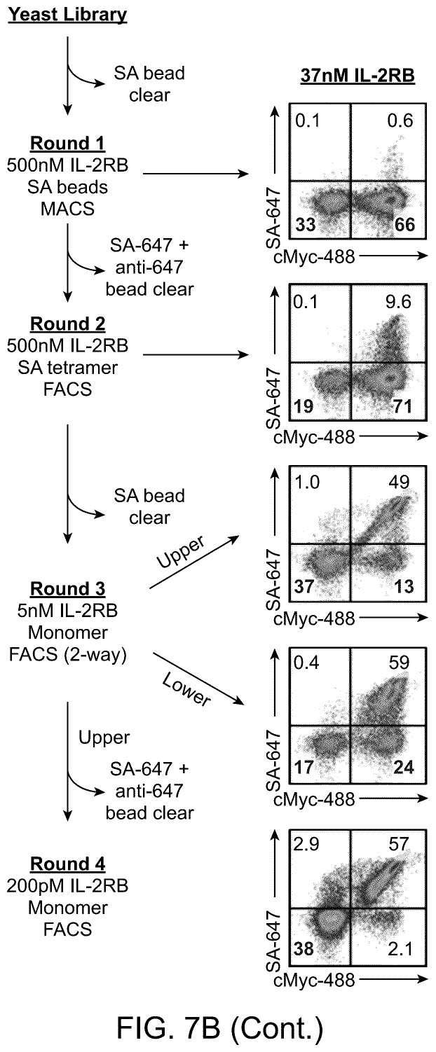

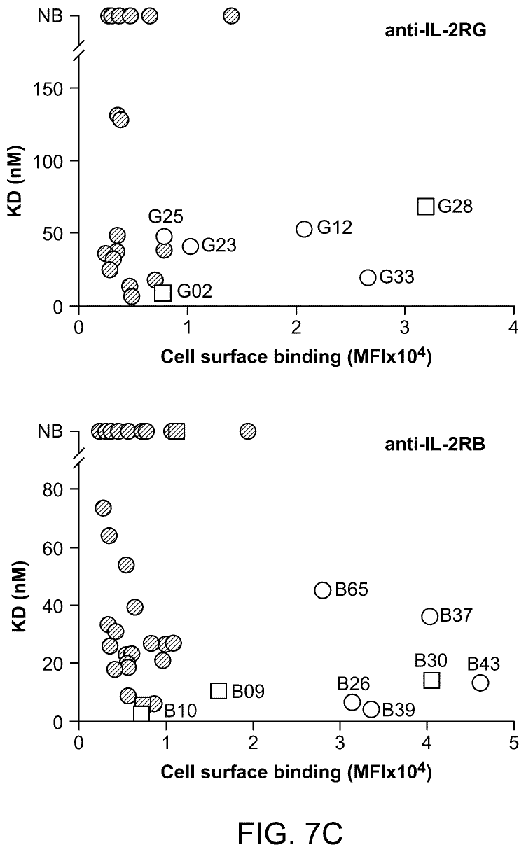

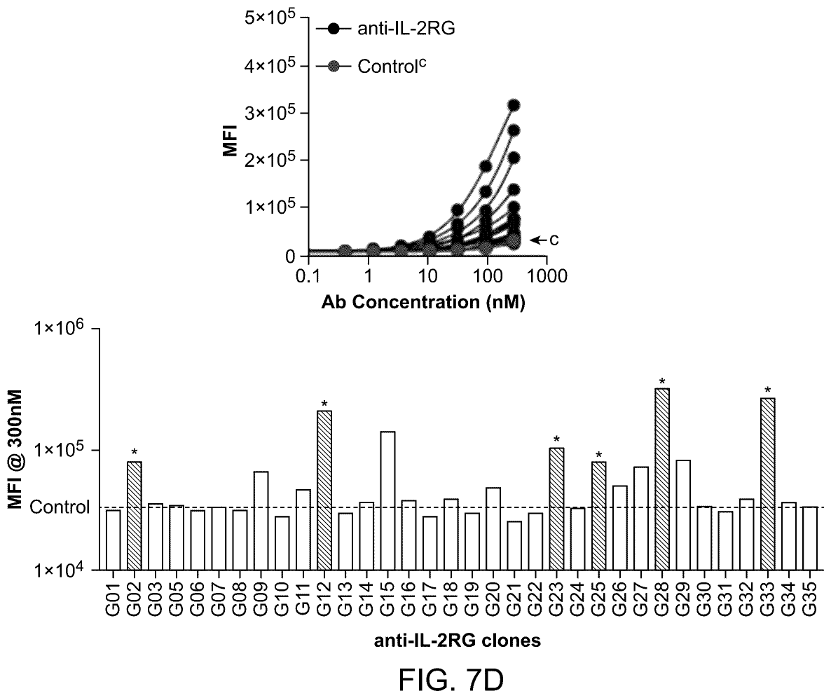

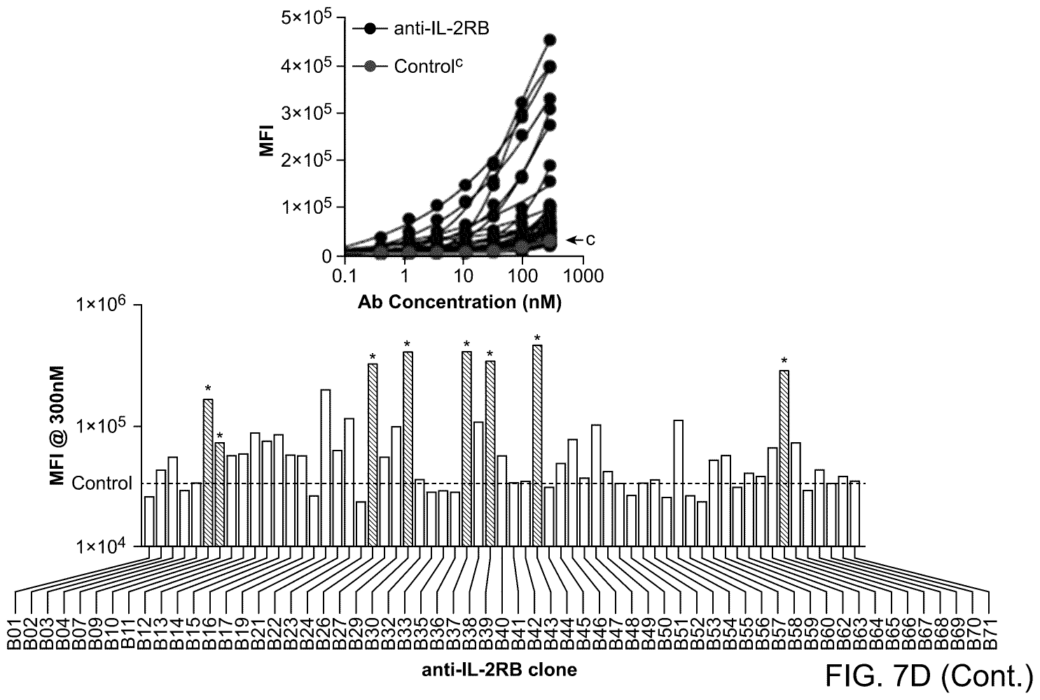

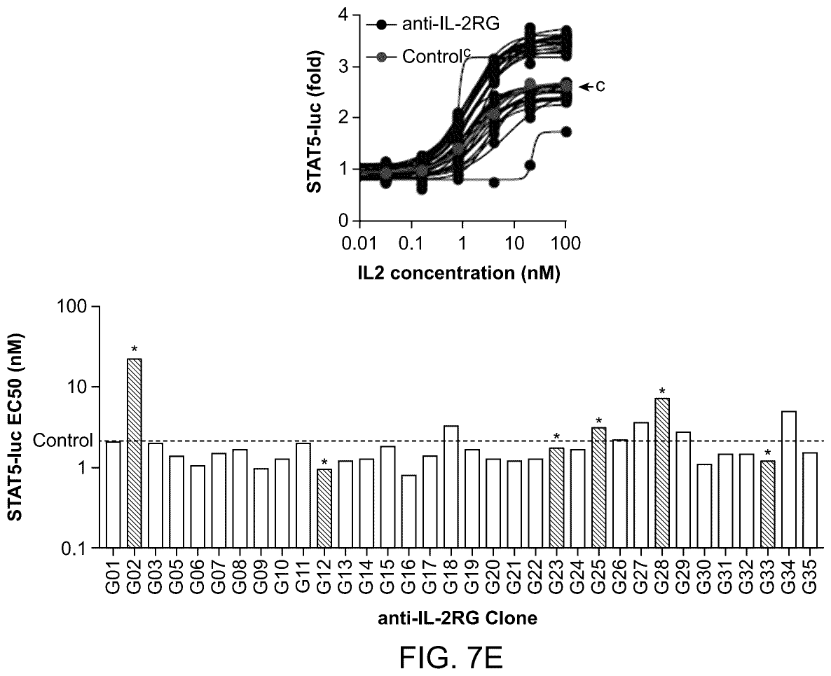

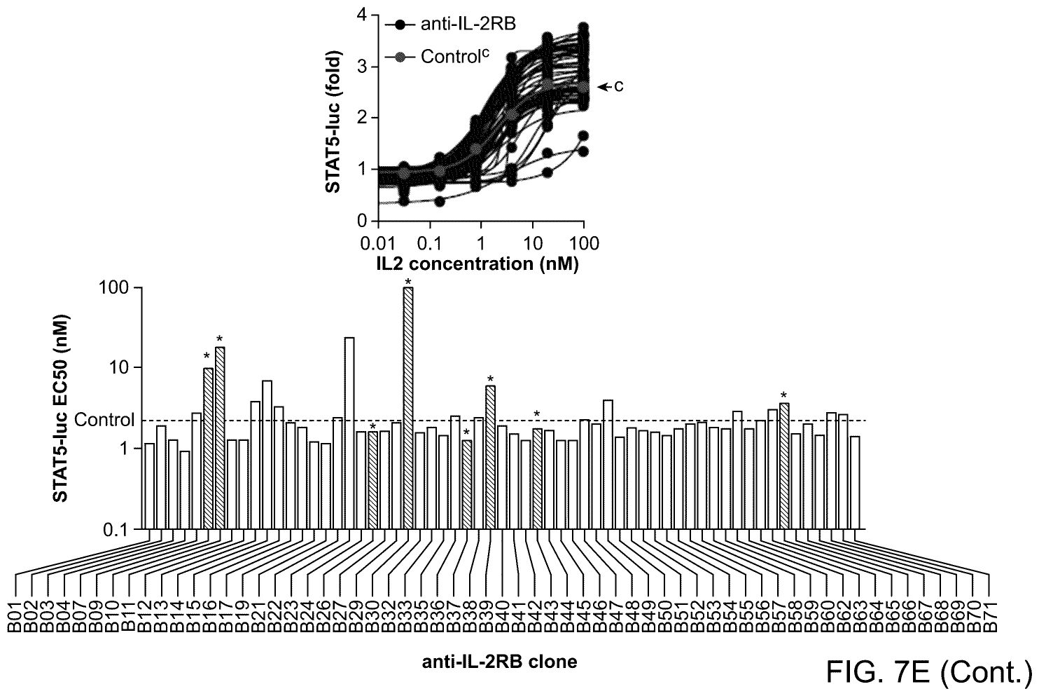

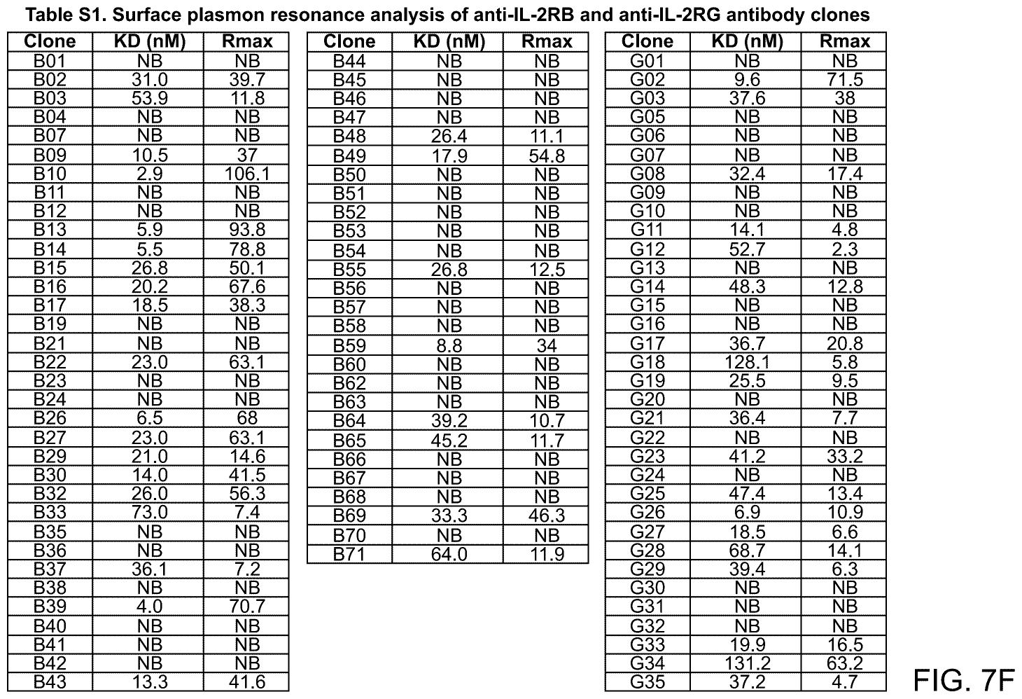

As a first step, antibody binders to IL-2RG and IL-2RB were discovered and characterized from an in-house naive scFv library displayed on yeast (FIG. 7A). Binders were selected using magnetic- and fluorescence-based sorting techniques with increasingly stringent conditions with regard to both antigen valency and concentration (FIG. 7B). After several rounds of selection and subsequent sequence analysis, 34 unique anti-IL-2RG and 61 unique anti-IL-2RB clones were selected for IgG reformatting and recombinant production. These clones were initially characterized by cell surface binding, SPR, and their ability to block IL-2 signaling (FIGS. 7C-7F). Based on these analyses, 8 anti-IL-2RB and 6 anti-IL-2RG antibodies were further characterized by epitope mapping and selected for bispecific assembly as WT IgG, contorsbody, and iAb formats.

### Example 8: Epitope Mapping of Anti-IL-2RG and Anti-IL-2RB Leads

While multiple techniques exist for the determination of antibody epitope, granular structural information was sought beyond epitope binning by cross-blocking. A mutational scanning technique was utilized in which an alanine was introduced at each residue of the his-tagged extracellular domains (ECDs) of IL-2RG and IL-2RB, resulting in 203 and 206 receptor mutants, respectively. These mutants were then arrayed on an SPR chip, and each lead clone was injected over the chip to determine which alanine mutants disrupted binding. When mapped onto the three-dimensional crystal structure of the receptors (PDB ID: 2ERJ) (Stauber, D. J., et al., Proc National Acad Sci, 2006. 103, 2788-2793), clustering of hits revealed the epitope of each clone.

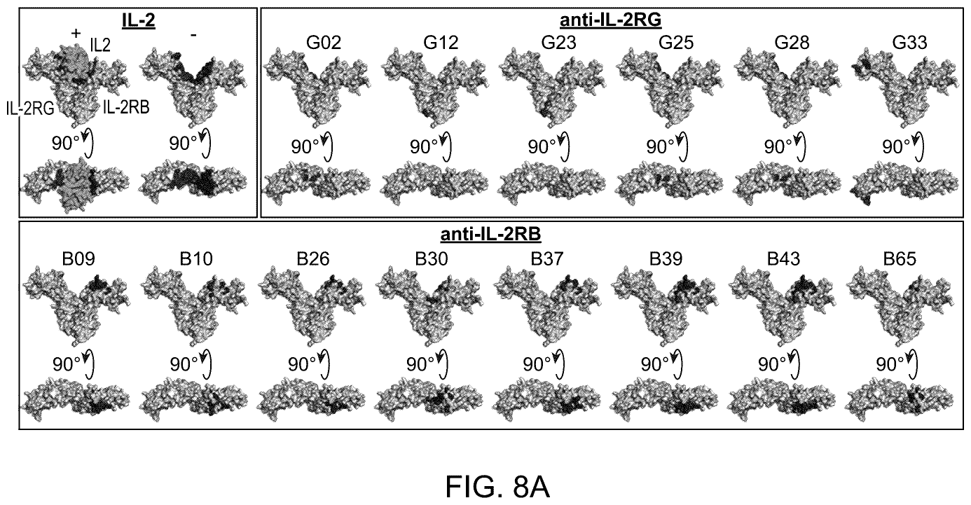

In relation to the IL-2 binding site that resides at the interface between IL-2RG and IL-2RB (FIG. 8A, black box), the epitopes of the anti-IL-2RG clones were dispersed across the receptor with some commonalities between clones (FIG. 8A, blue box). For example, clones G02, G25, and G28 overlapped with the IL-2 binding site, while G12 and G23 bound to the membrane proximal C-terminus, and G33 was the only clone that bound to the distal N-terminus. Conversely, all anti-IL-2RB clones had similar binding sites proximal to the binding site for IL-2 (FIG. 8A, red box). Clone B30 differed slightly in that its binding site almost completely overlapped with that of IL-2, which is consistent with its potent IL-2 blocking ability (FIGS. 8A and 7D-E).

### Example 9: Constrained Formats Enable IL-2 Pathway Agonism

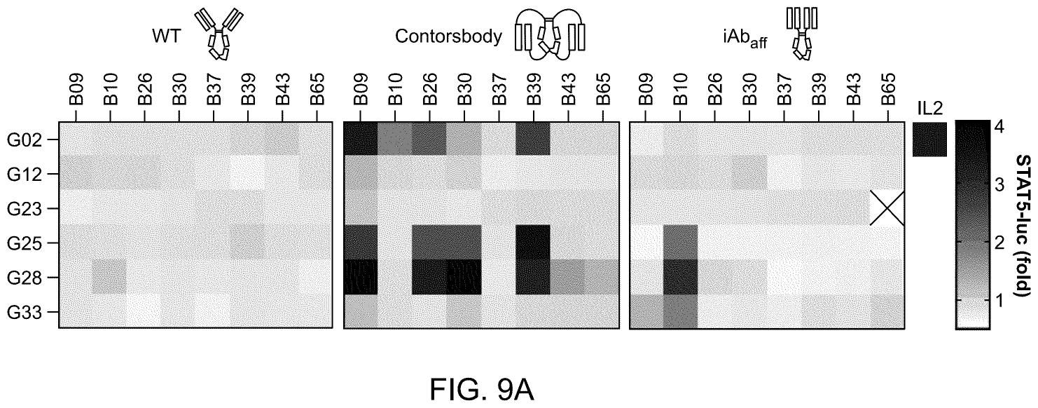

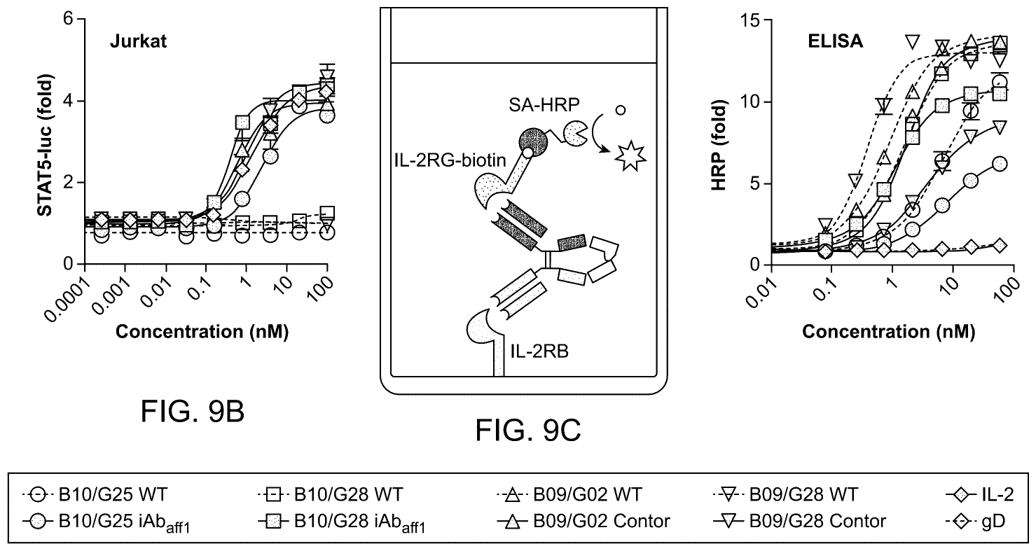

The 6 anti-IL-2RG and 8 anti-IL-2RB lead clones were reformatted as bispecific WT IgG, contorsbodies, and iAbs in a matrixed fashion where all IL-2RG clones were paired with all IL-2RB clones, resulting in 48 bispecific combinations for each format. Each bispecific antibody was tested for IL-2 pathway agonism using a Jurkat cell line engineered with a STAT5 luciferase reporter and both IL-2RG and IL-2RB overexpression. While no activity for WT IgG combinations was observed, multiple clone combinations were active for both the contorsbody and iAb constrained formats (FIG. 9A). Overall, the contorsbody format had a higher hit rate with 12/48 clones having greater than a 2-fold increase over the untreated control. There was a clonal and epitope dependence on activity, with active anti-IL-2RG clones targeting epitopes near the IL-2 binding site (FIG. 8A). It is unclear if this IL-2RG region is intrinsically favorable for antibody mimetic activity or if the increased activity of clones binding this region was biased due to the lack of epitope diversity within the anti-IL-2RB clones. For the iAb panel, only 3/47 clones were active using the same cutoff (G23/B65 did not express), and all 3 were combinations with a single IL-2RB clone, B10, despite the aforementioned epitope similarity among the IL-2RB clones. Interestingly, there was no overlap between active contorsbody and iAb clone combinations. Taken together, these results suggest that either iAb formation is most prominent for the B10 clone, and/or the apparent differing conformations accessed by the two formats are relevant for different Fab arm combinations and in a manner that is not based on epitope.

Two lead contorsbodies (B09/G02 and B09/G28) and 2 lead iAbs (B10/G25 and B10/G28) were selected to characterize further. First, titrations of each lead molecule in the Jurkat reporter assay were performed and compared the activity to that of both the WT IgG control molecules containing the same clone combinations and the IL-2 cytokine (FIG. 9B). Each constrained format had increased activity compared to the respective WT IgG control, and the activity of the lead engineered formats was comparable to that of IL-2. To determine whether the observed increase in agonism activity was simply due to an increased ability to bind simultaneously to the IL-2RG and IL-2RB receptors, we then performed a bridging enzyme linked immunosorbant assay (ELISA). For each clone combination, the constrained format showed a decreased ability to bind to both receptors simultaneously, suggesting that the enhanced activity of these formats is not due to increased binding but rather closer receptor proximity (FIG. 9C).

### Example 10: Constrained Antibody Agonists Mimic IL-2 Proliferative Activity and Transcriptional Reprogramming of Primary Cells

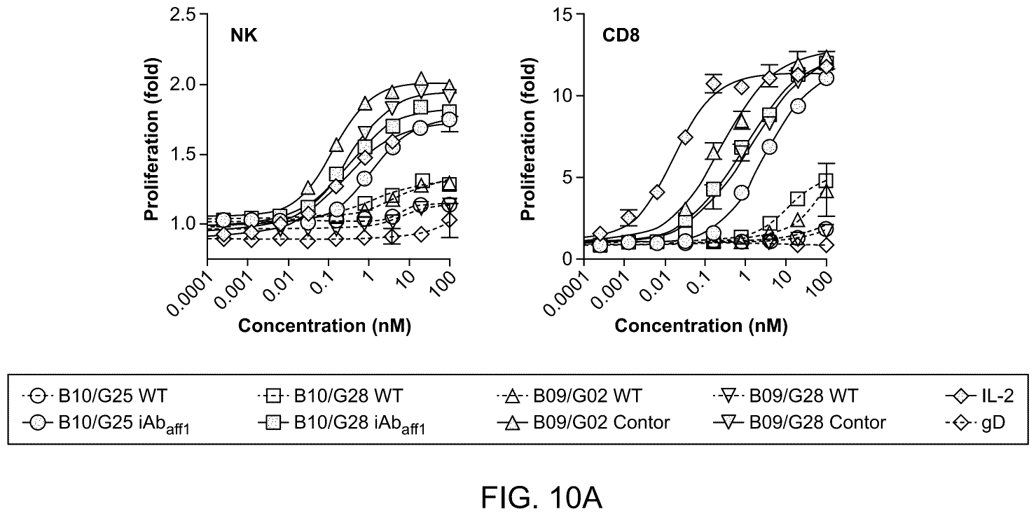

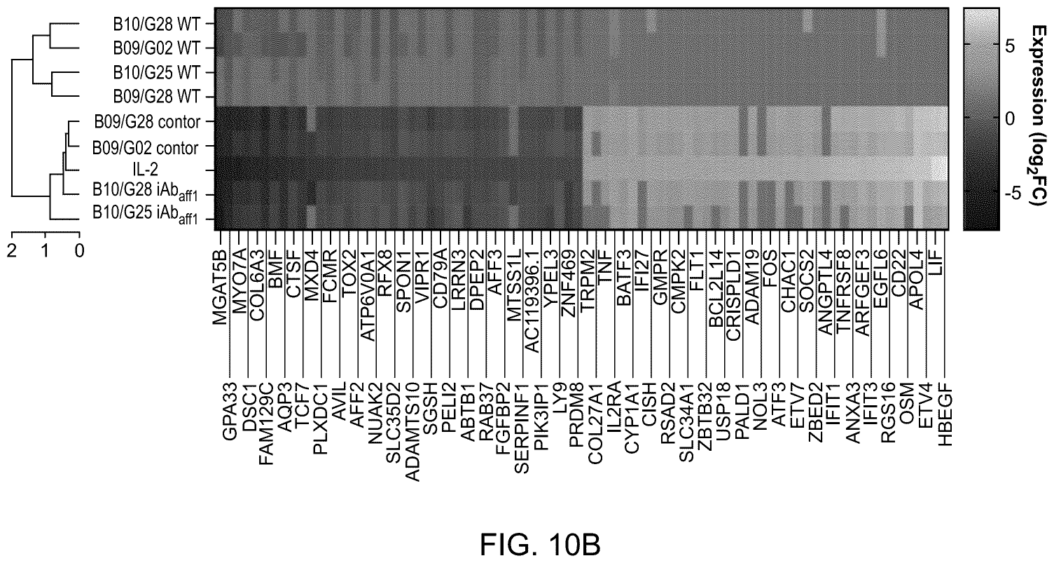

To more rigorously interrogate the activity of the top bispecific agonist antibodies and compare them to IL-2 cytokine in a more physiologically relevant cell type, titrations of the same lead clone combinations as above were performed in both primary NK and CD8 T cells, which endogenously express the IL-2 receptors. As was seen in the Jurkat reporter, all lead formats demonstrated increased activity over controls in both primary cell types (FIG. 10A). In contrast to both the Jurkat reporter and NK cells, recombinant IL-2 activity was significantly more potent than the mimetics on CD8 T cells, likely due to upregulation of IL-2RA on the surface of CD8 T cells upon CD3/CD28 stimulation which was performed prior to IL-2 treatment.

In order to determine the impact of the mimetic formats on transcriptional profiles, RNA sequencing was performed on CD8 T cells treated with lead IL-2 pathway agonists and their respective controls (FIG. 10B). Hierarchical clustering of normalized differentially expressed genes revealed two major groups of treatment conditions, with the lead constrained formats closely associated with IL-2 in one group and the WT IgG controls in a distinct group. A heat map of the 40 most down-regulated and up-regulated genes by IL-2 showed a strong overlap with the constrained formats, but not the WT IgG controls. Taken together, these data show that constrained antibody formats can be used to enable agonism of otherwise inactive bispecific antibody combinations in a manner that mimics the native ligand at both the proliferative and transcriptional level.

### Methods for the Above Examples

### Molecular Cloning

Antibody clones against each target were produced from various sources. anti-OX40, anti-4-1BB, anti-DR4, and anti-DR5 antibodies were discovered internally via mouse immunization campaigns. Sequences for all anti-CD40 antibodies used in this work were derived from publicly available databases and patent literature. The anti-IL-2RB and anti-IL-2RG antibodies used in this study were discovered using yeast display, as described below.

Gene fragments encoding all antibody constructs were synthesized as gBlocks or eBlocks (IDT) and cloned into the pRK mammalian expression vector using Gibson assembly (NEB, cat #E2611L). The pRK vector contains a cytomegalovirus (CMV) enhancer and promotor to control gene expression, an N-terminal secretion signal (MGWSCIILFLVATATGVHS (SEQ ID NO: 1)), a C-terminal simian virus 40 (SV40) PolyA sequence, and an ampicillin resistance gene for bacterial selection. Unless otherwise stated, all Fc regions were human IgG1 with the effectorless mutations L234A/L234A/P329G (EU numbering). The contorsbodies were constructed by fusing the heavy chain and light chain Fab domains to the N- and C-termini of the Fc domain, respectively, via flexible (G4S) 2 linkers (SEQ ID NO: 2), as previously described (14).

To make hexameric 3C8, heavy chain variable regions were cloned into an hIgG1 backbone containing E345R/E430G/S440Y (RGY) mutations (13, 17, 26). To promote formation of the h2B isoform of IgG2 for the anti-CD40 antibodies, the C131S mutation was used (EU numbering, corresponds to the C127S mutation of White and colleagues) (16). Fab constructs consisted of the light chain paired with a truncated heavy chain (1-225, EU numbering) and a C-terminal TEV protease-cleavable Flag tag. For all bispecific antibodies, knob-in-hole mutations were introduced into the Fc to enable heterodimerization (48).

OX40 ECD (L29-D170) was cloned into the pRK mammalian expression vector with a TEV protease-cleavable N-terminal His tag. IL-2RB ECD (A27-T240) and IL-2RG ECD (L23-A262) were cloned into the pRK mammalian expression vector with a C-terminal His tag.

### iAb Engineering

In this work, the iAb conformation was induced in antibodies of interest through engraftment of specific sets of mutations (FIGS. 1B and S1). The residue set used to induce domain exchange (iAbdx) was inspired by previous structural and mutational studies on the 2G12 antibody (8, 11), and the specific mutations with a representative example of the grafting approach can be found in FIGS. 2B and S1. The affinity interface iAb mechanism utilizes a hydrophobic patch on the surface of the heavy chain variable domain to facilitate intramolecular Fab-Fab association. The residue sets used to generate these Fab-Fab interactions and facilitate iAb formation (iAbaff1 and iAbaff2) were inspired by lineages of broadly neutralizing anti-HIV antibodies discovered in SHIV-infected macaques, specifically DH851 and DH898 (9). In order to graft each residue set onto “acceptor” antibody clones, we first aligned each antibody sequence and then substituted the amino acids at the given residues in FIG. 1B with the appropriate iAb inducing residue set, and a representative example of two anti-OX40 antibody grafts is depicted in FIG. S1. Based on varying degrees of amino acid conservation at each of the residues, grafting the residue set resulted in between 4 to 8 mutations per antibody, with an average of 7 mutations across all antibodies studied in this work.

### Protein Expression and Purification

With the exception of anti-DR4 and anti-DR5 antibodies, protein expression was performed by transfection of DNA into HEK293 cells. Anti-DR4 and anti-DR5 antibodies drive apoptosis of HEK293 cells and were therefore produced in CHO cells. For IgG and iAbs, co-transfection of heavy and light chain DNAs was performed. Since contorsbodies contain a genetic fusion of the light chain to the Fc region, only a single plasmid was required for monospecific formats. OX40 ECD was expressed with a baculovirus expression system in Tni insect cells in the presence of 10 mg/L kifunensine.

Following expression, affinity chromatography was performed using MabSelect SuRe resin (Cytiva, cat #17543803) for Fc-containing proteins, CaptureSelect CH1-XL resin (Thermo, cat #194346201L) for Fabs, and NiNTA agarose resin (Qiagen, cat #30210) for the receptor ECDs. Elution buffers consisted of 50 mM sodium citrate at pH 3.0 and 150 mM NaCl for the MabSelect SuRe and CaptureSelect CH1-XL resins, and 50 mM sodium phosphate at pH 7.4, 200 mM NaCl, and 400 mM imidazole for the NiNTA resin. Size exclusion chromatography was used as the final purification step using a HiLoad 16/600 Superdex 200 column. Protein quality was determined by analytical SEC using a Waters xBridge BEH200A SEC 3.5 um (7.8×300 mm) column (Waters, cat #176003596) and by SDS-PAGE. All antibody formats were stored in a buffer consisting of 20 mM histidine acetate and 150 mM NaCl at pH 5.5, while the receptor ECDs were stored in 25 mM tris pH 7.5 and 150 mM NaCl.

Bispecific IgG and iAb production were performed as previously described (49). In brief, half antibodies containing either the knob or hole mutations were first expressed in separate cell cultures and purified as described above. Two half antibodies were assembled into a single bispecific antibody through annealing, reduction, and oxidation steps. Size exclusion chromatography was used to separate the desired heterodimer species from unwanted homodimers. Due to the genetic fusion of the light chain, bispecific contorsbodies were produced in a single cell culture as described above without any in vitro assembly steps.

### Negative Stain Electron Microscopy

Antibody samples for negative stain EM analysis were exchanged into a buffer consisting of 25 mM tris and 150 mM NaCl, concentrated to 1 mg/ml, and filtered through 0.22 μm membranes (Costar, cat #8160). Samples were then diluted to 0.01 mg/ml, and 4 μl of the diluted sample was immediately deposited on a glow-discharged (Solarus plasma cleaner, Gatan) ultra-thin carbon coated 400-mesh copper grid (Electron Microscopy Sciences). After incubation for 30 seconds, the remaining liquid was blotted away with filter paper (Whatman, cat #WHA1001090), and the grid was washed 5× with 30 μl of filtered 2% uranyl acetate (Electron Microscopy Sciences). The excess uranyl acetate stain was blotted away with filter paper after 30 seconds. The grids were imaged on a Talos 200 C equipped with a 4K Ceta CMOS camera (ThermoFisher) at 73,000× magnification (2 Å per pixel). SerialEM was used for all data collection, and image processing was performed with cisTEM analysis software to generate 2D class averages. Percentages of i- and Y-shaped antibodies for a given sample were calculated using the number of particles in each 2D class.

### Generation of F(ab′)2

The F(ab′)2 construct of 3C8 iAbaff1 was generated through proteolytic cleavage of the lower hinge using a modified matrix metalloproteinase 3 (MMP3). The MMP3 protease was fused to the N-terminus of an in-house affinity matured anti-human Fc antibody based on the rheumatoid factor RF61 (50). Additionally, the MMP3 protease was engineered for more efficient activation through the addition of an enterokinase cleavage site within the pro-domain. Pro-domain cleavage and subsequent activation of the MMP3-antibody fusion construct was achieved by incubating 16 units of enterokinase (NEB, P8070L) for every 25 μg protein at room temperature for 16 hours in a buffer containing 25 mM tris at pH 7.5, 150 mM NaCl, and 10 mM CaCl2. To inactivate the enterokinase, 0.1 mg/ml soybean trypsin inhibitor (Sigma, 17075029) was added to the protein solution. The activated MMP3-antibody fusion was mixed with the 3C8 iAbaff1 construct at a 1:10 molar ratio and incubated overnight at 37° C. MabSelect SuRe resin was used to remove all cleaved Fc, unreacted IgG, and MMP3-antibody fusion protein, then the supernatant was purified with size exclusion chromatography and analyzed via SDS-PAGE.

### Affinity Measurements

Solution affinity constants for all antibodies were determined on a Biacore 8k+ or T200. Antibodies were diluted to 1 μg/ml in HBS-P+ buffer (Cytiva, cat #BR100671) and captured on a Series S Protein A chip (Cytiva, cat #29127555) according to the manufacturer's protocols. Serial dilutions of the appropriate receptor ECDs (recombinantly produced OX40, CD40, 4-1BB, DR4, DR5, IL-2RB, and IL-2RG, as described above) were prepared in HBS-P+. The dilutions were injected for 3 min, followed by a 5 min dissociation step. Affinity constants were determined from kinetic fits to the sensograms using the Biacore Evaluation Software.

### Cell Binding Analysis

A 0.6 μM solution of each anti-IL-2RB or anti-IL-2RG antibody was incubated overnight at 4° C. with 2.4 μM of Alexa Fluor 488 labeled anti-human IgG goat affiniPure fab fragment (Jackson, cat #109-547-008). Serial dilutions of each 4:1 molar ratio Fab: antibody mixture was then made in clear 384-well FACS plates in 20 μL FACS buffer (1×PBS with 1% BSA). 20 μL of FACS buffer containing 80,000 IL-2RB and IL-2RG overexpressing Jurkat cells was added to each well and incubated for 4 hours at 4° C. Cells were pelleted and washed 2 times, resuspended in 40 μL of FACS buffer, and analyzed on an iQue3 cytometer (Sartorius).

### Receptor-Mediated Internalization Assay

WT IgG, iAbaff1, or hexameric versions of the anti-OX40 clone, 3C8, were labeled with pHAb amine reactive dye according to the manufacture's protocols (Promega, cat #G9841). OX40 expressing Jurkat cells were treated with the indicated concentration of each pHAb labeled format for 1 hour at 37° C. and 5% CO2 in RPMI media containing 10% FBS and 2 mM L-glutamine (cRPMI). Cells were then washed twice with PBS containing 1% BSA and fluorescence was measured using the BL2 channel of a Sartorius iQue3.

Total internal reflection fluorescence (TIRF) microscopy and single particle tracking

Jurkat T cells were transfected (Amaxa) with 0.3 μg of an OX40-mNeongreen plasmid in a pRK vector backbone 48-72 hrs before live cell imaging. 48 well glass bottom plates were coated with 100 μg/mL poly-L-lysine for 30 minutes at 37 C, washed, and allowed to dry overnight before addition of anti-CD38 antibodies (OKT3 at 10 μg/mL) to stimulate T cells and enhance spreading. All imaging was performed on a Nikon TIRF system with a 100×1.49 NA objective, Hamatsu Orca FusionBT SCMOS camera, and iLas2 laser system for ellipse illumination to flatten the field at an imaging depth of 75-100 nm. After cell adhesion to surfaces the pre-treatment datasets were acquired at 20 Hz for 12.5 to 25 seconds (250-500 frames). The indicated anti-OX40 antibody formats were then added to the imaging wells at 2 ug/mL (13.3 nM) and the same pre-treated cells plus additional cells were acquired at the same frame-rate for the next 10 minutes. At least 6 cells per condition were analyzed from two independent experiments resulting in over 20,000 OX40 trajectories per condition. Tracking was performed with a DiaTrack 3.0 MatLab runtime application and mean square displacement plots were generated with custom written Igor track analysis code as previously described (51). Representative max projection images were created with imageJ and track insets were created with Igor and registered to the representative fields in Adobe Illustrator.

### Yeast Display

An in-house derived, S. cerevisiae yeast displayed scFv library containing a diversity of 1.6×109 unique sequences was used to select for binders to IL-2RG and IL-2RB using a combination of magnetic-activated cell sorting (MACS) and fluorescence-activated cell sorting (FACS) as depicted in FIG. S6. Briefly, yeast was electroporated with plasmid encoding the scFv library and grown to log phase at 30° C. in SDCAA media. Yeast representing 10× diversity of the library were grown at 20° C. in SGCAA media containing galactose for 24-48 hours at an OD600 of 1.0 prior to each round of selection. During each round of selection, scFv expression on the yeast surface was confirmed using an Alexa488-labeled anti-cMyc antibody (Cell Signaling) Binding to IL-2RG or IL-2RB was determined by the addition of the indicated concentration of in-house derived IL-2RB-biotin or commercially sourced IL-2RG-botin (Acro biosystems) and Alexa647-labeled streptavidin (SA) beads (MACS) or tetramers. For initial rounds of selection using magnetic SA beads, biotinylated antigen was mixed with the beads prior to addition of yeast to enhance avidity. For later rounds with tetrameric SA, yeast was first stained with biotinylated antigen, washed with PBS containing 1% BSA and then stained with SA. Each round was checked for enrichment of a binding population by staining yeast with a titration of antigen and analyzing fluorescence using an iQue3 (Sartorius). The results for 37 nM are shown in FIG. S6.

### Epitope Mapping

Epitope mapping of anti-IL-2RB and anti-IL-2RG clones was performed using a Carterra LSA. Alanine substitutions were first introduced at each residue of 6× histidine (SEQ ID NO: 3) tagged IL-2RB and IL-2RG ECDs using PCR based mutagenesis (n=206 and 203, respectively). If an alanine was already present the residue was mutated to glycine. Cystines were not mutated. All mutants were expressed in 293 cells and purified using NiNTA agarose resin as described above. Purified IL-2RB mutants were then arrayed and captured on a NiHC200M sensor chip for 5 min. A bispecific format where only one arm was specific for IL-2RB was flowed over the chip for 5 min and buffer was flowed for 5 min to allow for dissociation. The chip was then regenerated with 350 mM EDTA twice for 5 min and prepped with 5 mM NiCl for 5 min. This process was repeated for each of the anti-IL-2RB lead clones, and with the IL-2RG mutants combined with the anti-IL-2RG lead clones. Mutant receptor capture levels were calculated for each mutant at each cycle and response unit measurements were taken at the end of the association phase of each antibody. Ligand levels were plotted against antibody binding to identify alanine mutations that impacted antibody binding and these positions were highlighted on the previously reported crystal structure of the corresponding receptor (33).

### Bridging ELISA

Recombinantly expressed human IL-2RB was coated onto a Maxisorp 96-well plate (Thermo, cat #44-2404-21) overnight at 4° C. using a 1 μg/ml solution in PBS. The wells were then blocked with a solution of 0.5% BSA and 2 mM EDTA in PBS for 1 hour at room temperature. Three-fold dilutions of the lead antibodies were prepared in PBS with a top concentration of 60 μg/ml, and 100 μl of the antibody dilutions were added to the wells. The plates were incubated for 1 hour at room temperature and then washed 3 times with PBS. During the antibody incubation, a solution of 10 μg/ml biotinylated human IL-2RG (Acro Biosystems, cat #ILB-H82E3) and 100 μg/ml streptavidin-HRP (SouthernBiotech, cat #7100-05) was prepared in PBS and incubated at 37° C. for 30 minutes. The IL-2RG and streptavidin-HRP solution was diluted ten-fold, and 100 μl was added to the each washed Maxisorp well. After incubation for 30 minutes at 37° C., the plate was washed 3 times with PBS. 100 μl of TMB substrate (Thermo, cat #N301) was added to each well, and the absorbance at 650 nm was measured after 10 minutes. The absorbance signal was reported as fold-change over a control well without any added antibody.

### Cell-Based Reporter Assays

OX40, 4-1BB, DR4 and DR5 assays were performed as previously described (13, 17). For the CD40 bioassay, reporter cells were purchased from Promega (cat #JA2151) and used to assess CD40 agonist activity as follows. Cells were thawed and 10,000 cells were plated in each well of a black walled 384-well tissue culture treated plate (Corning, cat #3764) in 20 μL of cRPMI. Cells were allowed to adhere for 6 hours at 37° C. and 5% CO2. 20 μL of a serial dilution of the indicated antibody in cRPMI was then added to the cells and incubated overnight under the same conditions. The following day, 40 μL of Bright-Glo reagent (Promega, cat #E2650) was added to each well and luciferase signal was read on a Perkin Elmer Envision plate reader.

For the IL-2 reporter assay, Jurkat cells were engineered to express IL-2RB, IL-2RG and a STAT5-luciferase reporter construct. 20,000 cells in 20 μL of cRPMI were added to 20 μL of cRPMI containing a serial dilution of antibody or recombinant IL-2 and incubated overnight at 37° C. and 5% CO2. The following day, 40 μL of Bright-Glo reagent (Promega, cat #E2650) was added to each well and luciferase signal was read on a Perkin Elmer Envision plate reader. For IL-2 blocking experiments, cells were first coated with 1 μM of each monospecific anti-IL-2RG or anti-IL-2RB clone for 1 hour prior to the addition of an IL-2 serial dilution.

### Primary Cell Assays

Purified primary human CD8+ T cells (cat #70027) or NK cells (cat #70036) were purchased from STEMCELL technologies. CD8+ T cells were pre-stimulated with a 1:1 ratio of CD3/CD28 T-Activator Dynabeads (Gibco, cat #11131D) at 1×106 cells/mL in cRPMI at 37° C. and 5% CO2. After 48 hours the Dynabeads were magnetically separated from the cells and cells were allowed to rest overnight in cRPMI at 37° C. and 5% CO2. 50 μl or cRPMI containing 25,0000 cells was then added to 50 μL of cRPMI containing a serial dilution of the indicated mimetic antibody format or recombinant IL-2 and incubated at 37° C. and 5% CO2 in white 96-well plates (Corning, cat #3917). After 48 hours, 100 μL of CellTiter-Glo 2.0 (Promega, cat #G9242) was added to each well and luciferase signal was read on a Perkin Elmer Envision plate reader. The same protocol was followed for NK cells with the exception of the pre-stimulation step.

### RNA-Seq

Purified primary human CD8+ T cells (STEMCELL technologies, cat #70027) were pre-stimulated and rested as described above. 2×106 cells were plated in 2 mL of cRPMI in 6 well plates. Each well was treated in triplicate with 100 nM of the indicated mimetic antibody format or recombinant IL-2 and incubated at 37° C. and 5% CO2 for 24 hours. Cells were pelleted and RNA was extracted using a RNeasy mini kit (Qiagen, cat #74014).

Total RNA was quantified with Qubit RNA HS Assay Kit (Thermo Fisher Scientific) and quality was assessed using RNA ScreenTape on 4200 TapeStation (Agilent Technologies). For sequencing library generation, the Truseq Stranded mRNA kit (Illumina) was used with an input of 100 ng of total RNA. Libraries were quantified with Qubit dsDNA HS Assay Kit (Thermo Fisher Scientific) and the average library size was determined using D1000 ScreenTape on 4200 TapeStation (Agilent Technologies). Libraries were pooled and sequenced on NovaSeq 6000 (Illumina) to generate 30 million single-end 50-base pair reads for each sample.

RNA-sequencing data were analyzed using HTSeqGenie (52) in BioConductor (53) as follows: first, reads with low nucleotide qualities (70% of bases with quality <23) or matches to rRNA and adapter sequences were removed. The remaining reads were aligned to the human reference genome (human: GRCh38.p10) using GSNAP (54, 55) version ‘2013 Oct. 10-v2’, allowing maximum of two mismatches per 75 base sequence (parameters: ‘-M 2-n 10-B 2-i 1-N 1-w 200000-E 1--pairmax-rna=200000--clip-overlap’). Transcript annotation was based on the Gencode genes data base (human: GENCODE 27). To quantify gene expression levels, the number of reads mapping unambiguously to the exons of each gene was calculated.

Differential expression was calculated using EdgeR (56) (version 3.40.1), grouping on replicates of each condition relative to the negative control condition (gD). Differentially expressed genes were determined using a Benjamini-Hochberg False Discovery Rate of 1%. The log 2 fold-change of differentially expressed genes were normalized within each condition using sklearn.preprocessing.normalize (57) (scikit-learn version 1.0.1) before hierarchical clustering was performed using scipy.cluster.hierarchy (58) (SciPy version 1.7.3, parameters: method=‘ward’).

1. A human or humanized antibody comprising a first antigen binding domain comprising a first heavy chain variable (VH) domain and a first light chain variable (VL) domain, wherein the first VH and the first VL binds to a first target, wherein the VH domain comprises, according to Kabat numbering:
a) 7S, 17T, 19V, 21L, 68T, 70F, 77Q, 79I, 81I, and 82aT;
b) 7S, 17S, 19I, 21S, 68F, 70F, 77T, 79Y, 81V, and 82aS; or
c) 14A, 19I, 39R, 43G, 57R, 74L, 75E, 77F, 82aH, 82bK, 82 cM, 84V, and 113P, wherein the antibody does not bind to HIV.

2. A human or humanized antibody that is derived from a reference antibody, wherein the antibody and the reference antibody both comprise a first antigen binding domain comprising a first heavy chain variable (VH) domain and a first light chain variable (VL) domain, wherein:
a) the VH domain of the human or humanized antibody comprises, according to Kabat numbering, 1) at least one or more amino acid substitutions selected from the group consisting of 7S, 17T, 19V, 21L, 68T, 70F, 77Q, 79I, 81I, and 82aT, and 2) 7S, 17T, 19V, 21L, 68T, 70F, 77Q, 79I, 81I, and 82aT;
b) the VH domain of the human or humanized antibody comprises, according to Kabat numbering, 1) at least one or more amino acid substitutions selected from the group consisting of 7S, 17S, 19I, 21S, 68F, 70F, 77T, 79Y, 81V, and 82aS, and 2) 7S, 17S, 19I, 21S, 68F, 70F, 77T, 79Y, 81V, and 82aS; or
c) the VH domain of the human or humanized antibody comprises, according to Kabat numbering, 1) at least one or more amino acid substitutions selected from the group consisting of 14A, 19I, 39R, 43G, 57R, 74L, 75E, 77F, 82aH, 82bK, 82 cM, 84V, and 113P, and 2) 14A, 19I, 39R, 43G, 57R, 74L, 75E, 77F, 82aH, 82bK, 82 cM, 84V, and 113P,
wherein the substitutions in a), b), and c) are substitutions compared to the reference antibody; wherein optionally the human or humanized antibody has increased agonistic activity relative to the reference antibody.

3. The human or humanized antibody of claim 2, wherein the human or humanized antibody does not bind to HIV.

4. The human or humanized antibody of any one of claims 1-3, wherein the first VH domain of the human or humanized antibody comprises, according to Kabat numbering, 7S, 17T, 19V, 21L, 68T, 70F, 77Q, 79I, 81I, and 82aT.

5. The human or humanized antibody of any one of claims 1-3, wherein the first VH domain of the human or humanized antibody comprises, according to Kabat numbering, 7S, 17S, 19I, 21S, 68F, 70F, 77T, 79Y, 81V, and 82aS.

6. The human or humanized antibody of any one of claims 1-3, wherein the first VH domain of the human or humanized antibody comprises, according to Kabat numbering, 14A, 19I, 39R, 43G, 57R, 74L, 75E, 77F, 82aH, 82bK, 82 cM, 84V, and 113P.

7. The human or humanized antibody of any one of claims 2-4, wherein the first VH of the human or humanized antibody comprises at least one or more substitutions at the position(s) selected from 19, 21, 70, 79, and 81.

8. The human or humanized antibody of any one of claims 2-3 and 5, wherein the first VH of the human or humanized antibody comprises at least one or more substitutions at the position(s) selected from 19, 68, 70, and 81.

9. The human or humanized antibody of any one of claims 2-3 and 6, wherein the first VH of the human or humanized antibody comprises at least one or more substitutions at the position(s) selected from 14, 19, 39, 43, 74, 77, 82a, and 82b.

10. The human or humanized antibody of any one of claims 1-9, wherein the antibody is a monovalent antibody.

11. The human or humanized antibody of claim 10, wherein the monovalent antibody is a Fab.

12. The human or humanized antibody of any one of claims 1-9, wherein the antibody is a F(ab′)₂.

13. The human or humanized antibody of any one of claims 1-12, wherein the antibody does not have a Fc domain.

14. The human or humanized antibody of any one of claims 1-10, wherein the antibody has a Fc domain.

15. The human or humanized antibody of any one of claims 1-10 and 14, wherein the antibody is an IgG antibody.

16. The human or humanized antibody of any one of claims 1-16, wherein the antibody has a modified hinge, wherein the modified hinge confers more restricted hinge flexibility.

17. The human or humanized antibody of any one of claims 1-16, wherein the human or humanized antibody is a monospecific antibody.

18. The human or humanized antibody of any one of claims 1-17, wherein the human or humanized antibody binds to a cell surface receptor.

19. The human or humanized antibody of claim 18, wherein the human or humanized antibody activates a target via receptor clustering.

20. The human or humanized antibody of any one of claims 1-19, wherein the human or humanized antibody binds to one or more TNFRSF member.

21. The human or humanized antibody of claim 20, wherein the human or humanized antibody binds to OX40, CD40, 4-1BB, DR4, or DR5.

22. The human or humanized antibody of claim 21, wherein the human or humanized antibody binds to CD40, optionally wherein the human or humanized antibody is derived from an antibody selected from the group consisting of ravagalimab, dacetuzumab, giloralimab, and sotigolimab.

23. The human or humanized antibody of claim 21, wherein the human or humanized antibody binds to OX40, optionally wherein the human or humanized antibody is derived from an antibody selected from the group consisting of 3C8, 1A7, 2A3, 2B5, 2F10, 2G7, 2H5, 3F5, 3G5, and 3G8.

24. The human or humanized antibody of any one of claims 1-17, wherein the human or humanized antibody binds to a receptor of a cytokine.

25. The human or humanized antibody of claim 24, wherein the cytokine can form a complex in nature with at least two distinct receptors, which triggers downstream activity of the cytokine.

26. The human or humanized antibody of claim 24 or claim 25, wherein the human or humanized antibody binds to an IL-2 receptor.

27. The human or humanized antibody of claim 26, wherein the IL-2 receptor is IL-2RG or IL-2RB.

28. The human or humanized antibody of any one of claims 1-9 and 12-27, wherein the human or humanized antibody is a bivalent antibody comprising a second antigen binding domain comprising a second VH domain and a second VL domain binding to a second target.

29. The human or humanized antibody of claim 28, wherein the second VH domain comprises, according to Kabat numbering:
a) 7S, 17T, 19V, 21L, 68T, 70F, 77Q, 79I, 81I, and 82aT;
b) 7S, 17S, 19I, 21S, 68F, 70F, 77T, 79Y, 81V, and 82aS; or
c) 14A, 19I, 39R, 43G, 57R, 74L, 75E, 77F, 82aH, 82bK, 82 cM, 84V, and 113P.

30. The human or humanized antibody of claim 28 or claim 29, wherein the second target is distinct from the first target.

31. The human or humanized antibody of any one of claims 27-30, wherein the human or humanized antibody binds to both IL-2RG and IL-2RB.

32. The human or humanized antibody of any one of claims 27-31, wherein the VH domain of the human of humanized antibody comprises 3 VH CDR sequences of B10, and wherein the VL domains comprises 3 VL CDR sequences of B10.

33. The human or humanized antibody of claim 31 or 32, wherein one of the two VH domains comprises 3 VH CDR sequences of B10, one of the two VL domains comprises 3 VL CDR sequences of B10, and wherein the other of the two VH domains comprises 3 VH CDR sequences of G25 or G28, the other of the two VL domains comprises 3 VL CDR sequences of G25 or G28.

34. A pharmaceutical composition comprising the antibody of any one of claims 1-33 and a pharmaceutical career.

35. An isolated nucleic acid encoding the antibody of any one of claims 1-33 or a fragment thereof.

36. A host cell comprising the nucleic acid of claim 35.

37. A method of producing the antibody of any one of claims 1-33 or a fragment thereof comprising culturing the host cell of claim 36 under conditions suitable for the expression of the antibody or a fragment thereof.

38. The method of claim 37, further comprising recovering the antibody or a fragment thereof from the host cell.

39. A method of producing an agonist antibody from a reference antibody, comprising substituting one or more amino acid residues on a heavy chain variable (VH) domain of the reference antibody to promote an i-shaped antibody format.

40. A method of producing an agonist antibody from a reference antibody, comprising:
a) substituting one or more amino acid residues on a heavy chain variable (VH) domain of the reference antibody at position(s) according to Kabat numbering selected from 7, 17, 19, 21, 68 70, 77, 79, 81, and/or 82a, wherein the agonist antibody has a VH domain comprising 7S, 17T, 19V, 21L, 68T, 70F, 77Q, 79I, 81I, and 82aT after substitution;
b) substituting one or more amino acid residues on a heavy chain variable (VH) domain of the reference antibody at position(s) according to Kabat numbering selected from 7, 17, 19, 21, 68 70, 77, 79, 81, and/or 82a, wherein the agonist antibody has a VH domain comprising 7S, 17S, 19I, 21S, 68F, 70F, 77T, 79Y, 81V, and 82aS after substitution; or
c) substituting one or more amino acid residues on a heavy chain variable (VH) domain of the reference antibody at position(s) according to Kabat numbering selected from 14, 19, 39, 43, 57, 74, 75, 77, 82a, 82b, 82c, 84, and 113, wherein the agonist antibody has a VH domain comprising 14A, 19I, 39R, 43G, 57R, 74L, 75E, 77F, 82aH, 82bK, 82 cM, 84V, and 113P after substitution.

41. An agonist antibody produced by a method of any one of claims 37-40.

42. A method of promoting agonistic activity of an antibody, comprising:
a) substituting one or more amino acid residues on a heavy chain variable (VH) domain of the antibody at position(s) according to Kabat numbering selected from 7, 17, 19, 21, 68 70, 77, 79, 81, and/or 82a, wherein the VH domain after substitution comprises 7S, 17T, 19V, 21L, 68T, 70F, 77Q, 79I, 81I, and 82aT;
b) substituting one or more amino acid residues on a heavy chain variable (VH) domain of the antibody at position(s) according to Kabat numbering selected from 7, 17, 19, 21, 68 70, 77, 79, 81, and/or 82a, wherein the VH domain after substitution comprises 7S, 17S, 19I, 21S, 68F, 70F, 77T, 79Y, 81V, and 82aS; or
c) substituting one or more amino acid residues on a heavy chain variable (VH) domain of the antibody at position(s) according to Kabat numbering selected from 14, 19, 39, 43, 57, 74, 75, 77, 82a, 82b, 82c, 84, and 113, wherein the VH domain after substitution comprises 14A, 19I, 39R, 43G, 57R, 74L, 75E, 77F, 82aH, 82bK, 82 cM, 84V, and 113P.

43. The antibody of any one of claims 1-33 or the pharmaceutical composition of claim 34 for use as a medicament.

44. The antibody of any one of claims 1-33 or the pharmaceutical composition of claim 34 for use in treating a disease or condition.

45. Use of the antibody of any one of claims 1-33 or the pharmaceutical composition of claim 34 in the manufacture of a medicament for treating a disease or condition.

46. A method of treating an individual having a disease or condition comprising administering to the individual an effective amount of the antibody of any one of claims 1-33 or the pharmaceutical composition of claim 34.

47. A library comprising polynucleotides, wherein the polynucleotides in the library encode at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering:
a) 7S, 17T, 19V, 21L, 68T, 70F, 77Q, 79I, 81I, and 82aT;
b) 7S, 17S, 19I, 21S, 68F, 70F, 77T, 79Y, 81V, and 82aS; or
c) 14A, 19I, 39R, 43G, 57R, 74L, 75E, 77F, 82aH, 82bK, 82 cM, 84V, and 113P.

48. A library of antibodies, comprising at least two, at least three, at least four, at least five, or at least ten unique antibodies, wherein each of these antibodies comprises a heavy chain variable (VH) domain and a light chain variable (VL) domain, wherein the VH and the VL binds to a target, wherein the VH domain comprises, according to Kabat numbering:
a) 7S, 17T, 19V, 21L, 68T, 70F, 77Q, 79I, 81I, and 82aT;
b) 7S, 17S, 19I, 21S, 68F, 70F, 77T, 79Y, 81V, and 82aS; or
c) 14A, 19I, 39R, 43G, 57R, 74L, 75E, 77F, 82aH, 82bK, 82 cM, 84V, and 113P.

49. The library of claim 47 or claim 48, wherein the antibodies are expressed or to be expressed on the surface of one or more phages or yeast cells.

50. A method of screening an agonist antibody comprising contacting the antibodies in the library of claim 48 or claim 49 with the target or with a cell expressing the target.

51. The method of claim 50, wherein the method further comprises assessing agonistic activity of the antibodies.


SEQUENCE LISTING (14 sequences)

SEQ ID NO: 1 - protein, 19 aa, synthetic construct
MGWSCIILFLVATATGVHS

SEQ ID NO: 2 - protein, 10 aa, synthetic construct
GGGGSGGGGS

SEQ ID NO: 3 - protein, 6 aa, synthetic construct
HHHHHH

SEQ ID NO: 4 - protein, 123 aa, Homo sapiens
EVQLVESGGGLVKAGGSLILSCGVSNFRISAHTMNWVRRVPGGGLEWVSSISSSSSYIYYADSVKGRFTVSRDDLEDFVYLQMHKMRVEDTAIYYCARKGSDRLSDNDPFDAWGPGTVVTVSP

SEQ ID NO: 5 - protein, 117 aa, Homo sapiens
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 6 - protein, 117 aa, synthetic construct
EVQLVQSGAEVKKAGASVIVSCKASGYTFTDSYMSWVRRAPGGGLEWIGDMYPDNGDISYNQKFRERVTITRDTLESFAYLELHKMRVEDTAVYYCVLAPRWYFSVWGQGTLVTVSP

SEQ ID NO: 7 - protein, 114 aa, Homo sapiens
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDRLDYWGQGTLVTVSS

SEQ ID NO: 8 - protein, 114 aa, synthetic construct
EVQLVQSGAEVKKAGASVIVSCKASGYAFTNYLIEWVRRAPGGGLEWIGVINPGSGDIYYSEKFKGRVTLTADTLESFAYLELHKMRVEDTAVYYCARDRLDYWGQGTLVTVSP

SEQ ID NO: 9 - protein, 123 aa, Macaca mulatta
QVTLMESGPALVKVTQTLAVTCTFSGFSIRDSGKGVAWIRQPPGGALEWLTSIYWDDTKYHDTSLKPRLTIFRDTSQTQVILILTNMAPLDTATYYCGRINNGGGWKDHIDFWGPGLLVTVSS

SEQ ID NO: 10 - protein, 117 aa, synthetic construct
EVQLVQSGAEVKKPGATVVVLCKASGYTFTDSYMSWVRQAPGQGLEWIGDMYPDNGDSSYNQKFRERVTIFRDTSTSQAILILTSLRSEDTAVYYCVLAPRWYFSVWGQGTLVTVSS

SEQ ID NO: 11 - protein, 114 aa, synthetic construct
EVQLVQSGAEVKKPGATVVVLCKASGYAFTNYLIEWVRQAPGQGLEWIGVINPGSGDTYYSEKFKGRVTLFADTSTSQAILILTSLRSEDTAVYYCARDRLDYWGQGTLVTVSS

SEQ ID NO: 12 - protein, 120 aa, Macaca mulatta
QDLLLQSGAEVREPGASVTVSCQASNYTFPDYTIHWVRLVPGQGLEWLGEMKPKVGVTNVSKKIRDRLFMTADTSTDTAYMVLSALTPGDTAIYYCTRLEPDFLSGWAHWGKGVLVTVSP

SEQ ID NO: 13 - protein, 117 aa, synthetic construct
EVQLVQSGAEVKKPGASVIVSCKASGYTFTDSYMSWVRQAPGQGLEWIGDMYPDNGDSSYNQKFRERVFIFRDTSTSTAYLVLSSLRSEDTAVYYCVLAPRWYFSVWGQGTLVTVSS

SEQ ID NO: 14 - protein, 114 aa, synthetic construct
EVQLVQSGAEVKKPGASVIVSCKASGYAFTNYLIEWVRQAPGQGLEWIGVINPGSGDTYYSEKFKGRVFLFADTSTSTAYLVLSSLRSEDTAVYYCARDRLDYWGQGTLVTVSS